(19) United States
(12) Patent Application Publication
(10) Pub. No.: US 2026/0308681 A1
(43) Pub. Date: Oct. 8, 2026

(54) COMPOSITIONS AND METHODS FOR ASSESSING NEUROINFLAMMATION USING SOMATIC MUTATIONS

(71) Applicant: The Children's Medical Center Corporation, Boston, MA (US)

(72) Inventors: Yue Huang, Boston, MA (US); Zinan Zhou, Boston, MA (US); Eunjung A. Lee, Boston, MA (US); Christopher A. Walsh, Boston, MA (US)

(73) Assignee: The Children's Medical Center Corporation, Boston, MA (US)

(21) Appl. No.: 19/183,128

(22) Filed: Apr. 18, 2025

Related U.S. Application Data
(63) Continuation of application No. PCT/US2023/077378, filed on Oct. 20, 2023.
(60) Provisional application No. 63/418,317, filed on Oct. 21, 2022.

(51) Int. Cl. C12Q 1/6883; C12Q 1/6869; G16B 20/20; G16B 30/10

(52) U.S. Cl. CPC C12Q 1/6883; C12Q 1/6869; G16B 20/20; G16B 30/10; C12Q 2600/156; C12Q 2600/16

(57) ABSTRACT

This disclosure relates generally to compositions and methods for assessing neuroinflammation based on the presence of absence of somatic mutations. The compositions and methods provided herein are useful for predicting a subject's risk of chronic neuroinflammation. In addition, this disclosure provides therapeutic agents that can be used to reduce or prevent neuroinflammation.

### CROSS REFERENCE TO RELATED APPLICATIONS

The present application is a continuation under 35 U.S.C. § 111(a) of PCT International Patent Application No. PCT/US2023/077378, filed Oct. 20, 2023, designating the United States and published in English, which claims priority to and the benefit of U.S. Provisional Application No. 63/418,317, filed Oct. 21, 2022, which is hereby incorporated by reference in its entirety.

### STATEMENT OF RIGHTS TO INVENTIONS MADE UNDER FEDERALLY SPONSORED RESEARCH

This invention was made with government support under Grant/Contract Numbers W81XWH-20-1-0028, awarded by the Department of the Army, and NS035129, AG051791, and AG065502 awarded by the National Institutes of Health. The government has certain rights in the invention.

### BACKGROUND OF THE INVENTION

Alzheimer's disease (AD) is an age-associated neurodegenerative disorder characterized by progressive neuronal loss and the pathological accumulation of the misfolded proteins amyloid-0 and tau. Neuroinflammation, the activation of the brain's innate immune system, is potentially one of the early pathogenic processes occurring in Alzheimer's disease, starting long before symptoms appear.

Somatic mutations have been found to accumulate in all cell types that have been studied, both during normal development and during the aging process. Clonal expansion, driven by somatic mutations in genes regulating cell proliferation, is considered the major cause of cancer but has also been recently reported in various non-cancer cell types often in the absence of visible pathology. Clonal expansion of mutant blood cells, so-called clonal hematopoiesis of indeterminate potential (CHIP), increases in prevalence with age and is associated with increased risk of hematologic malignancies and cardiovascular disease, perhaps through inflammatory effects of mutant cells on the neighboring nonmutant cells.

Current methods of distinguishing Alzheimer's disease from other forms of dementia, neurodegeneration, or cognitive impairment are inadequate. Accordingly, improved methods for characterizing Alzheimer's disease are urgently required.

### SUMMARY OF THE INVENTION

This disclosure provides compositions and methods for characterizing and treating neuroinflammation, dementia, and cognitive impairment. In embodiments, methods described herein use the detection of somatic mutations in a biological sample derived from a subject to detect the presence of Alzheimer's disease (AD) or other neurodegenerative disorders in the subject.

In one aspect, the present disclosure provides a method of characterizing neuroinflammation in a biological sample of a subject. The method involves: a) sequencing a set of proliferation-related or cell cycle-related polynucleotides to generate sequencing reads, where each polynucleotide includes a unique molecular identifier (UMIs), and where the polynucleotides are derived from a biological sample of a subject comprising somatic cells; b) generating a consensus sequence for each group of sequencing reads including the same UMI; c) comparing the consensus sequence to a reference sequence to identify somatic single nucleotide variations (sSNVs) and somatic indels in the consensus sequence that are not present in the reference sequence, where the presence of sSNVs and somatic indels in the consensus sequence is indicative of neuroinflammation in the biological sample.

In another aspect, the present disclosure provides a computer implemented method of determining a burden of somatic mutations in a biological sample of a subject. The method involves: a) sequencing RNA present in a biological sample including somatic cells of the subject to generate sequencing data; b) aligning a set of RNA sequences in the sequencing data against a human reference genome to obtain alignment reads; c) filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample; and d) calculating the burden of somatic mutations in the biological sample relative to the reference sequence.

In another aspect, the present disclosure provides a method of characterizing Alzheimer's disease in a biological sample of a subject. The method involves: a) deep DNA panel sequencing a set of proliferation-related or cell cycle-related polynucleotides to generate sequencing reads, where each polynucleotide includes a unique molecular identifier (UMIs), and where the polynucleotides are derived from a biological sample including somatic cells; b) generating a consensus sequence for each group of sequencing reads including the same UMI; c) comparing the consensus sequence to a reference sequence to identify somatic single nucleotide variations (sSNVs) and somatic indels in the consensus sequence that are not present in the reference sequence, where the presence of sSNVs and somatic indels in the consensus sequence relative to a reference is indicative of Alzheimer's disease or a propensity to develop Alzheimer's disease in the subject and the absence of sSNVs and somatic indels in the consensus sequence relative to a reference is indicative that the subject does not have Alzheimer's disease or a propensity to develop Alzheimer's disease.

In another aspect, the present disclosure provides a method of characterizing Alzheimer's disease in a biological sample of a subject. The method involves: a) sequencing RNA present in a biological sample including somatic cells of the subject to generate sequencing data; b) aligning a set of RNA sequences in the sequencing data against a human reference genome to obtain alignment reads; c) filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample; and d) calculating the burden of somatic mutations in the biological sample relative to the reference sequence, wherein an increase in the burden of somatic mutations is indicative that the subject has or has a propensity to develop Alzheimer's disease.

In another aspect, the present disclosure provides a method of characterizing neuroinflammation in a subject. The method involves: bulk sequencing RNA from a biological sample of a subject to identify somatic mutations in expressed genes; deep sequencing genomic DNA from a biological sample of the subject to identify somatic single-nucleotide variations in the genomic DNA; and determining the number of somatic single-nucleotide variations identified by bulk sequencing RNA and deep sequencing of genomic DNA relative to a reference, where increased somatic single-nucleotide variations in RNA and DNA relative to the reference indicates neuroinflammation.

In another aspect, the present disclosure provides a method of characterizing Alzheimer's disease in a subject. The method involves: a) bulk sequencing RNA from a fluid sample of a subject to obtain sequencing data; b) deep sequencing genomic DNA from a fluid sample of the subject to obtain sequencing data; c) analyzing the sequencing data relative to a reference sequence to identify somatic single-nucleotide variations in the RNA and genomic DNA; wherein increased somatic single-nucleotide variations relative to the reference indicates chronic neuroinflammation.

In another aspect, the present disclosure provides a panel of capture molecules for detection of an sSNV, sIndel, or mutation burden in a gene, where the panel of capture molecules includes one or more of: AKT3, AKT1, ASXL1, ATRX, BCR, CBL; CHEK2, CHK2, CX3CR1, DEPDC5; DNMT3A, EPPK1, KMT2D, MED12, MLH1, MS4A7 MRC1, MTOR, PDGFRA, PI3K, PIK3CA, PIK3R1, PPM1D, P2RY12, STAT3, TSC1, TET2, TMEM119, and TP53, where the capture molecule is a polynucleotide or polypeptide.

In another aspect, the present disclosure provides a set of probes targeting the exons and exon-intron junctions of proliferation-related genes listed in Table 1.

In another aspect, the present disclosure provides a set of primers for amplifying the gene panels of any one of the above aspects, or embodiments thereof.

In another aspect, the present disclosure provides a method of reducing neuroinflammation in a subject. The method involves contacting a cell of the subject with an agent of Table 1 that inhibits the activity or expression of a proliferation-related polypeptide, cell cycle-related polypeptide, tumor suppressor polypeptide, or a polynucleotide encoding such polypeptides.

In another aspect, the present disclosure provides a method of characterizing neuroinflammation in a biological sample of a subject. The method involves: a) sequencing RNA present in a biological sample comprising somatic cells of the subject to generate sequencing data; b) analyzing the sequencing data using the computer, in comparison to a reference sequence, at one or more loci associated with clonal hematopoiesis of indeterminate potential (CHIP), to generate a set of somatic copy number variations associated with CHIP present in the somatic cells; and c) calculating a burden of somatic copy number variations associated with CHIP in the biological sample relative to a reference subject, where an increase in the burden of somatic copy number variations in the subject, relative to the reference sequence, is indicative of neuroinflammation in the biological sample.

In another aspect, the present disclosure provides a computer implemented method of determining a burden of somatic copy number variations in a biological sample of a subject. The method involves: a) sequencing RNA present in a biological sample comprising somatic cells of the subject to generate sequencing data; b) analyzing the sequencing data using the computer, in comparison to a reference sequence, at one or more loci associated with CHIP, to generate a set of somatic copy number variations associated with CHIP present in the somatic cells; and c) calculating a burden of somatic copy number variations associated with CHIP in the biological sample relative to the reference sequence.

In another aspect, the present disclosure provides a method of characterizing Alzheimer's disease in a biological sample of a subject. The method involves: a) sequencing RNA present in a biological sample comprising somatic cells of the subject to generate sequencing data; b) analyzing the sequencing data using the computer, in comparison to a reference sequence, at one or more loci associated with CHIP, to generate a set of somatic copy number variations associated with CHIP present in the somatic cells; and c) calculating a burden of somatic copy number variations associated with CHIP in the biological sample relative to a reference subject, where an increase in the burden of somatic copy number variations in the subject, relative to the reference sequence, is indicative of Alzheimer's disease or a propensity to develop Alzheimer's disease in the subject.

In another aspect, the present disclosure also provides a method of characterizing neuroinflammation in a biological sample of a subject. The method involves: a) sequencing RNA present in a biological sample comprising somatic cells of the subject to generate sequencing data; b) analyzing the sequencing data using the computer, in comparison to a reference sequence, at one or more loci associated with CHIP, to generate a set of somatic copy number variations associated with CHIP present in the somatic cells; c) sorting the sequencing data, using the set of somatic copy number variations associated with CHIP, into sequencing data from cells having copy number variations associated with CHIP (sCNV data) and sequencing data from cells not having copy number variations associated with CHIP (no sCNV data); and d) comparing expression levels for a set of genes between the sCNV data and the no SCNV data, where the set of genes include one or more of NEAT1, CD163, SLC11A1, SRGN, ASAP1, CADM1, RGS1, HIF1A, and GLUL, and where an increase in expression levels for the set of genes in the sCNV data as compared to the no sCNV data is indicative of neuroinflammation in the biological sample.

In another aspect, the present disclosure provides a method of characterizing Alzheimer's disease in a biological sample of a subject. The method involves: a) sequencing RNA present in a biological sample comprising somatic cells of the subject to generate sequencing data; b) analyzing the sequencing data using the computer, in comparison to a reference sequence, at one or more loci associated with CHIP, to generate a set of somatic copy number variations associated with CHIP present in the somatic cells; c) sorting the sequencing data, using the set of somatic copy number variations associated with CHIP, into sequencing data from cells having copy number variations associated with CHIP (sCNV data) and sequencing data from cells not having copy number variations associated with CHIP (no sCNV data); and d) comparing expression levels for a set of genes between the sCNV data and the no SCNV data, where the set of genes comprises one or more of NEAT1, CD163, SLC11A1, SRGN, ASAP1, CADM1, RGS1, HIF1A, and GLUL, and where an increase in expression levels for the set of genes in the sCNV data as compared to the no sCNV data is indicative of Alzheimer's disease or a propensity to develop Alzheimer's disease in the subject.

In another aspect, the present disclosure provides a kit for characterizing neuroinflammation in a biological sample of a subject. The kit includes a panel of capture molecules of any one of the above aspects, or embodiments thereof.

In any of the above aspects, or embodiments thereof, the method further involves de-convoluting sequencing data and comparing it against a reference set of cell-type specific sequencing data to calculate the proportion of cell types present in the sample.

In any of the above aspects, or embodiments thereof, the method further involves generating a consensus read sequence from multiple reads derived from an original polynucleotide fragment, where each of the multiple reads comprises a UMI that is also present in the original polynucleotide fragment.

In any of the above aspects, or embodiments thereof, consensus reads supported by fewer than two reads on both strands are excluded.

In any of the above aspects, or embodiments thereof, the method further involves filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample

In any of the above aspects, or embodiments thereof, the average depth of sequencing is at least 1000×.

In any of the above aspects, or embodiments thereof, the average coverage is at least 80% of a targeted region at 500× for consensus reads.

In any of the above aspects, or embodiments thereof, the biological sample is derived from a subject having or suspected of having a neuroinflammatory condition.

In any of the above aspects, or embodiments thereof, the neuroinflammatory condition is a neurodegenerative disease.

In any of the above aspects, or embodiments thereof, the neurodegenerative disease is Alzheimer's disease, Parkinson's Disease, Huntington's Disease, Fronto-temporal Dementia, Amyotrophic Lateral Sclerosis (ALS), HIV-related dementia, Progressive Supranuclear Palsy (PSP), Pick's disease, Post-COVID encephalopathy, Chronic Traumatic Encephalopathy (CTE), and Lewy Body Disease (LBD).

In any of the above aspects, or embodiments thereof, the method further involves de-convoluting sequencing data and comparing it against a reference set of cell-type specific sequencing data to calculate the proportion of cell types present in the sample.

In any of the above aspects, or embodiments thereof, the method further involves generating a consensus read sequence from multiple reads derived from an original polynucleotide fragment, where each of the multiple reads comprises a UMI that is also present in the original polynucleotide fragment.

In any of the above aspects, or embodiments thereof, the average sequencing depth is greater than 1000× after UMI collapsing.

In any of the above aspects, or embodiments thereof, the biological sample is a tissue sample or liquid sample.

In any of the above aspects, or embodiments thereof, the liquid sample is a blood or cerebrospinal fluid sample.

In any of the above aspects, or embodiments thereof, the biological sample includes a blood cell, a neuron, a microglial cell, a CNS-associated macrophage (CAM), astrocyte, oligodendrocyte, endothelial cell, pericyte, monocyte, or cell free DNA derived from any of the aforementioned cell types.

In any of the above aspects, or embodiments thereof, the neuron or microglial cell is derived from prefrontal cortex, temporal cortex, or another brain region.

In any of the above aspects, or embodiments thereof, the method detects somatic mutations with mutant allele fractions of less than about 3%.

In any of the above aspects, or embodiments thereof, the method detects somatic mutations with mutant allele fractions of less than about 1%.

In any of the above aspects, or embodiments thereof, the method detects somatic mutations with mutant allele fractions of less than about 0.5%.

In any of the above aspects, or embodiments thereof, the method detects somatic mutations with mutant allele fractions of less than about 0.1%.

In any of the above aspects, or embodiments thereof, deep sequencing is performed on a polynucleotide comprising a tumor suppressor gene or RNA transcribed from a tumor suppressor gene.

In any of the above aspects, or embodiments thereof, the reference is a DNA or RNA sequence derived from a corresponding healthy control subject.

In any of the above aspects, or embodiments thereof, the reference is the sequence of a proliferation-related gene, cell cycle-related gene, tumor suppressor gene or RNA transcribed from such genes derived from a corresponding healthy control subject.

In any of the above aspects, or embodiments thereof, the sSNV, sIndel, or mutation burden is detected in a gene selected from those listed in Table 4.

In any of the above aspects, or embodiments thereof, the sSNV, sIndel, or mutation burden is detected in a gene, where the gene is one or more of: AKT3, AKT1, ASXL1, ATRX, BCR, CBL; CHEK2, CHK2, CX3CR1, DEPDC5; DNMT3A, EPPK1, KMT2D, MED12, MLH1, MS4A7 MRC1, MTOR, PDGFRA, PI3K, PIK3CA, PIK3R1, PIK3R2, PPM1D, P2RY12, STAT3, TSC1, TET2, TMEM119, and TP53.

In any of the above aspects, or embodiments thereof, the sSNV, sIndel, or mutation burden is detected in a panel of genes, where the panel of genes is one or more of:
TET2, ASXL1, KMT2D, ATRX, and CBL;
CX3CR1, TMEM119, and P2RY12;
MS4A7 and MRC1;
DNMT3A and TET2;
DNMT3A, PPM1D, TET2, CBL, TET2, TP53, FGFR1;
PIK3CA, MTOR, PIK3R1, PIK3R2, TSC1, AKT3, AKT1, TSC2, STK11, and DEPDC5;
CX3CR1, TMEM119, P2RY12; DNMT3A, TET2, ASXL1, KMT2D, ATRX, BCR, CBL, TP53, MLH1, and STAT3.

In any of the above aspects, or embodiments thereof, the sSNV, sIndel, or mutation burden is detected in a gene, where the gene is one or more of EPPK1, PPM1D, TP53, PDGFRA, TET2, ASXL1, MED12, and CHK2.

In any of the above aspects, or embodiments thereof, the sIndel is one or more of EPPK1 (2 bp insertion), PPM1D (10 bp deletion) TP53 (1 bp deletion), PDGFRA (4 bp insertion), TET2 (1 bp deletion), ASXL1 (1 bp insertion), MED12 (24 bp deletion), CHEK2 (1 bp deletion), and TET2 (1 bp insertion).

In any of the above aspects, or embodiments thereof, the sSNV, sIndel, or mutation burden is detected in a gene selected from those genes listed in Table 1.

In any of the above aspects, or embodiments thereof, the sSNV, sIndel, or mutation burden is detected in a Phosphoinositide 3-kinase (PI3K) pathway polynucleotides, where the PI3K pathway polynucleotide is one or more of: PTEN, mTOR, PI3KCA, PIK3R2, TSC1, TSC2, DEPDC5, NPRL3, NPRL4, or another component of the PI3K pathway.

In any of the above aspects, or embodiments thereof, the sSNV is a splice-site sSNV in DNMT3A (c.1429+1G>A), FGFR1 (p.Arg506Gln), In any of the above aspects, or embodiments thereof, the sSNV is a deleterious missense sSNVs in TET2 (p.Pro1194Ser and p.Val1371Asp).

In any of the above aspects, or embodiments thereof, an AD microglia includes reduced expression of CX3CR1 and P2RY12.

In any of the above aspects, or embodiments thereof, the sSNV, sIndel, or mutation burden is detected in TET2, ASXL1, KMT2D, ATRX, and/or CBL.

In any of the above aspects, or embodiments thereof, an AD microglia includes increased expression of a gene of Table 4.

In any of the above aspects, or embodiments thereof, the gene includes a C to A, C to G, C to T, T to A, or T to G mutation.

In any of the above aspects, or embodiments thereof, there is at least about a 10% or greater increase in sSNVs and/or sIndels detected in a biological sample from a subject having or having a propensity to develop Alzheimer's disease or another neurodegenerative condition.

In any of the above aspects, or embodiments thereof, the increase is at least about a 25% increase.

In any of the above aspects, or embodiments thereof, there is at least about a 2× or 5× increase in sSNVs and/or sIndels detected in a biological sample from a subject having or having a propensity to develop Alzheimer's disease or another neurodegenerative condition.

In any of the above aspects, or embodiments thereof, the sSNV is a mutation where the mutation is one or more of: a missense mutation, nonsense mutation, in-frame deletion, in-frame insertion, frameshift deletion, frameshift insertion, and splice site mutation.

In any of the above aspects, or embodiments thereof, the detection of the sSNV, sIndel, or mutation burden involves RNA-seq, next generation sequencing, panel sequencing, amplicon sequencing, single-cell/single-nuclei RNA-seq, single-cell/single-nuclei ATAC-seq, Nano-seq, META-CS, or other duplex sequencing methods.

In any of the above aspects, or embodiments thereof, the panel includes capture molecules each of which specifically binds one or more of the following genes:
TET2, ASXL1, KMT2D, ATRX, and CBL;
CX3CR1, TMEM119, and P2RY12;
MS4A7 and MRC1;
DNMT3A and TET2;
DNMT3A (C.1429+1G>A), PPM1D (Leu484fs), TET2 (Val1371Asp), CBL (Leu380Pro), TET2 (Pro1194Ser), TP53 (Arg280Gly), FGFR1 (Arg473Gln);
PIK3CA, MTOR, PIK3R1, TSC1, AKT3, AKT1, TSC2, STK11, and DEPDC5;
CX3CR1, TMEM119, P2RY12; DNMT3A, TET2, ASXL1, KMT2D, ATRX, BCR, CBL, TP53, MLH1, and STAT3.

In any of the above aspects, or embodiments thereof, the panel includes capture molecules each of which specifically binds one or more of the following genes: EPPK1, PPM1D, TP53, PDGFRA, TET2, ASXL1, MED12, and CHK2.

In any of the above aspects, or embodiments thereof, the panel includes capture molecules each of which specifically binds one or more of the following sIndels EPPK1 (2 bp insertion), PPM1D (10 bp deletion) TP53 (1 bp deletion), PDGFRA (4 bp insertion), TET2 (1 bp deletion), ASXL1 (1 bp insertion), MED12 (24 bp deletion), CHEK2 (1 bp deletion), and TET2 (1 bp insertion).

In any of the above aspects, or embodiments thereof, the panel includes capture molecules each of which specifically binds a gene selected from those genes listed in Table 1 or Table 4.

In any of the above aspects, or embodiments thereof, the panel includes capture molecules each of which specifically binds one or more of: PTEN, mTOR, PI3K, or another component of the PI3K pathway).

In any of the above aspects, or embodiments thereof, the panel includes capture molecules each of which specifically binds one or more of: a splice-site sSNV in DNMT3A (c.1429+1G>A) or FGFR1 (p.Arg506Gln), In any of the above aspects, or embodiments thereof, the panel includes capture molecules each of which specifically binds one or more of: a deleterious missense sSNVs in TET2 (p.Pro1194Ser and p.Val1371Asp).

In any of the above aspects, or embodiments thereof, the panel includes capture molecules each of which specifically binds TET2, ASXL1, KMT2D, ATRX, and/or CBL.

In any of the above aspects, or embodiments thereof, the capture molecule is a primer, probe, or aptamer.

In any of the above aspects, or embodiments thereof, the agent inhibits the activity or expression of a PTEN, mTOR, PI3K polypeptide, or another component of the PI3K pathway

In any of the above aspects, or embodiments thereof, the method further involves comparing the sequencing data against a reference set of cell-type specific sequencing data to sort the sequencing data into sets of cell-type specific sequencing data.

In any of the above aspects, or embodiments thereof, the analyzing in step involves analyzing the sorted cell-type specific sequencing data, and comparing the sorted cell-type specific sequencing data, based on cell type, to the reference set of cell-type specific sequencing data.

In any of the above aspects, or embodiments thereof, the biological sample includes a microglia-perivascular macrophage, astrocyte, oligodendrocyte, oligodendrocyte precursor cell, excitatory neuron, or cell free DNA derived from any of the aforementioned cell types.

In any of the above aspects, or embodiments thereof, the one or more loci associated with CHIP are one or more of the regions disclosed in Table 6.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change in the structure, expression levels or activity of a gene or polypeptide as detected by standard art known methods such as those described herein. In the context of expression or activity an alteration may be an increase or decrease. As used herein, an alteration includes a 10% change in expression or activity levels, preferably a 25% change, more preferably a 40% change, and most preferably a 50% or greater change in expression levels. In embodiments, an alteration is a mutation in a gene (e.g., tumor suppressor gene).

In this disclosure, “comprises,” “comprising,” “containing,” and “having” and the like can have the meaning ascribed to them in U.S. Patent law, and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “capture molecule” is meant a reagent that specifically binds a nucleic acid molecule or polypeptide to select or isolate the nucleic acid molecule or polypeptide. In embodiments, the capture molecule is a polynucleotide (e.g., primer, probe, oligonucleotide) bound to a substrate (e.g., bead, membrane, array).

By “clonal hematopoiesis of indeterminate potential (CHIP)” is meant the presence of a clonally expanded hematopoietic stem cell caused by a mutation in individuals without evidence of hematologic malignancy, dysplasia, or cytopenia. CHIP is associated with a 0.5-1.0% risk per year of leukemia. In general, CHIP involves a condition in which somatic mutations are found in cells of the blood or bone marrow, but no other criteria for hematologic neoplasia are met.

By “copy number variant” or “copy number variation” (CNV) is meant a phenomenon in which sections of the genome are repeated and the number of repeats in the genome varies between individuals. CNVs occur without any limitations of frequency and may arise in somatic cells, e.g., a “somatic copy number variant (sCNV).”

By “deep sequencing” is meant sequencing a region of a polynucleotide tens or hundreds or even thousands of times. In some embodiments, deep sequencing includes next-generation sequencing, high-throughput sequencing and massively parallel sequencing. Deep sequencing involves obtaining large numbers of sequences corresponding to relatively short, targeted regions of a genome. A targeted region can include, for example, an entire gene or a portion of a gene (such as a mutation hotspot), or a regulator of the gene (e.g., a promoter or enhancer). In some embodiments, many thousands of clonal sequences are obtained from a short, targeted segment allowing identification and quantitation of somatic single nucleotide variants. In some embodiments, a particular region of a polynucleotide is sequenced for example 100, 250, 500, 1,000, 2,500, 5,000, 7,500, 10,000, 25,000, 50,000, 100,000, 250,000, 500,000, 750,000, or even 1, 5, or 10, 25, 50, 75, or 100 million times.

By “detect,” is meant any method for identifying the presence, absence, or amount of a single nucleotide variation.

By “disease” is meant any condition or disorder that damages or interferes with the normal function of a cell, tissue, or organ. Examples of diseases include, without limitation, diseases and disorders associated with an accumulation of somatic mutations and/or age-related onset, including but not limited to neuroinflammatory disorders, including but not limited to neurodegenerative disorders, such as Alzheimer's disease, Parkinson's disease, Huntington's Disease, Fronto-Temporal Dementia (FTD), HIV-related dementia, Post-COVID encephalopathy, Chronic Traumatic Encephalopathy (CTE), Lewy Body Disease (LBD), Amyotrophic Lateral Sclerosis (ALS), Pick's disease, Progressive Supranuclear Palsy (PSP), and mixed forms of dementia.

The terms “isolated,” “purified,” or “biologically pure” refer to material that is free, to varying degrees, from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “gene” refers to a nucleic acid molecule or portion of a nucleic acid molecule comprising a sequence that encodes a protein. It is understood in the art that a gene also comprises non-coding sequences, such as 5′ and 3′ flanking sequences (such as promoters, enhancers, repressors, and other regulatory sequences) as well as introns.

As used herein, the term “mutation” is meant to include any genetic alteration. Genetic alterations may occur in a protein coding or in a regulatory sequence. Exemplary mutations include point mutations and small insertion/deletion mutations (e.g., 1-50-bp insertion or deletion mutation). In embodiments, mutations can lead to changes in the structure of an encoded protein or to a decrease or complete loss in its expression.

As used herein, the term “nucleic acid molecule” refers to a polymeric form of nucleotides. Exemplary polynucleotides include ribonucleotides or deoxyribonucleotides (e.g., RNA, DNA, and combinations or analogs thereof.

“Primer set” means a set of oligonucleotides that may be used for DNA amplification, for example, PCR. A primer set would consist of at least 2, 4, 6, 8, 10, 12, 14, 16, 18, 20, 30, 40, 50, 60, 80, 100, 200, 250, 300, 400, 500, 600, or more primers.

By “reference” is meant a standard or control condition. In one embodiment, the sequence of a gene in a subject having or having a propensity to develop neuroinflammation or neurodegeneration, such as Alzheimer's or Parkinson's disease, is compared to a reference sequence, such as the sequence of a healthy control subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween. In one embodiment, a reference sequence is a sequence from a healthy control subject. In some embodiments, a reference sequence is a sequence from a cell in a healthy subject which lacks a somatic mutation (e.g., a somatic single nucleotide variation or a somatic single copy number variation) or a consensus sequence from many cells in a single healthy subject, or a consensus sequence from many cells from many healthy subjects.

As used herein, the term “sequencing” and its variants comprise obtaining sequence information from a nucleic acid strand, typically by determining the identity of at least some nucleotides (including their nucleobase components) within the nucleic acid molecule. The term sequencing may also refer to determining the order of nucleotides (base sequences) in a nucleic acid sample, e.g., DNA or RNA. Many techniques are available such as Sanger sequencing and high-throughput sequencing technologies (also known as next-generation sequencing technologies) such as the GS FLX platform offered by Roche Applied Science, based on pyro sequencing.

The terms “somatic mutation,” and “somatic single-nucleotide variation,” refer to an alteration in a polynucleotide sequence of a somatic cell that may or may not be shared by other cells on the basis of their derivation from a common progenitor cell.

By “somatic copy number variation (sCNV)” is meant an alteration in the number of repeats in the genome of a somatic cell that may or may not be shared by other cells on the basis of their derivation from a common progenitor cell. sCNVs can include the gain or loss of whole chromosomes (aneuploidy) or of chromosomal segments.

By “somatic mutational burden” is meant a measure of the number of somatic mutations within a cell.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal (e.g., a bovine, equine, canine, ovine, feline, rodent, or primate).

The term “variant” is used herein to refer to a change or alteration in sequence relative to a reference sequence at a particular locus. In embodiments the alteration or variant is a nucleotide base substitution, deletion, or insertion in coding or non-coding regions.

The term “single nucleotide variant,” or “single nucleotide variation,” (SNV) refers to a single nucleotide alteration at a particular site. SNVs occur without any limitations of frequency and may arise in somatic cells, e.g., a “somatic single-nucleotide variant (sSNV).” In various embodiments, the sSNV is identified by the presence of a complementary nucleotide (G-C; A-T) on the opposite strand.

The term “single nucleotide polymorphism” (SNP) refers to a single nucleotide alteration at a particular site that occurs in at least about 1% of the general population of a species. In the human genome, single nucleotide polymorphisms occur about once in every 300 nucleotide base pairs. SNPs may or may not be located within genes and may or may not affect gene expression or protein function.

By “unique molecular identifier” or “UMI” is meant a short nucleic acid sequence that is identifiable in, for example, high-throughput sequencing techniques. In embodiments, the sequencing method is single-cell RNA-seq.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C provide an overview of the experimental and analysis strategies for detecting somatic single nucleotide variations. FIG. 1A provides a schematic diagram showing the transcriptome-wide screen of somatic mutations among 886 bulk RNA-seq data sets of AD and control brain samples. Somatic mutations were called by RNA-MosaicHunter. FIG. 1B provides a schematic showing workflow for the profiling of somatic single-nucleotide variants (sSNVs) and sIndels in 342 AD and control prefrontal cortex (PFC) samples using deep molecular barcode sequencing with a panel of 149 proliferation-related genes (Table 4). Mutation candidates were validated by amplicon sequencing and their mutant allele fractions were measured in different fluorescence-activated nuclei sorting (FANS) sorted nuclei populations. FIG. 1C provides a schematic diagram showing a workflow for the identification and transcriptomic profiling of microglia in AD and control brain single-nucleus RNA-seq samples carrying somatic copy number variants at loci associated with CHIP (CHIP-sCNV). Throughout the application the term “SEA-AD” denotes the Seattle Alzheimer's Disease Brain Cell Atlas.

FIGS. 2A-2F provide a scatter-plot, bar graphs, and a plot showing that RNA-MosaicHunter revealed elevated burden of somatic mutations in the cerebral cortex of AD patients. FIGS. 2A-2B provide a scatter-plot and bar graph showing benchmarking of the performance of RNA-MosaicHunter using the TCGA cancer data. 513 of 613 sSNVs identified by RNA-MosaicHunter were confirmed by Mutect in the matched DNA-seq data (FIG. 2A). RNA-MosaicHunter recaptured 65 sSNVs that are present in DNA-seq but missed by Mutect (grey bar in bottom panel). TP: true positive; FN: false negative; FP: false positive. FIGS. 2C-2D provide bar graphs showing a greater mutation burden in cerebral cortex samples of AD patients when compared to matched controls. A significant two-fold increase of sSNV density in AD prefrontal cortex and temporal cortex was consistently found in both ROSMAP (FIG. 2C) and MayoRNAseq (FIG. 2D) cohorts. The burden increase was not observed in the AD cerebellum. CI, cognitive impairment. FIG. 2E provides a plot showing that linear regression modeling confirms that the sSNV increase in AD brains remains significant after controlling for potential covariates. PMI, post-mortem interval. FIG. 2F provides a bar graph showing the top 10 Gene Ontology terms enriched for AD sSNVs. Genes regulating cell cycle and proliferation are specifically enriched for AD but not control sSNVs. FIGS. 2C-2E, Error bar, 95% CI.

FIGS. 3A-3G provide bar graphs, a plot, a mutation map, a schematic, and box plots showing elevated burdens of somatic mutations in proliferation-related genes in AD brains. FIGS. 3A-3B provide bar graphs showing AD prefrontal cortex samples harbor significantly more sSNVs in 149 targeted genes than matched controls, using both the sSNV list of stringent (FIG. 3A) and sensitive (FIG. 3B) identification pipelines. The sensitive list additionally contains recurrent sSNVs if they were specifically enriched in the AD or control groups. FIG. 3C provides a plot showing that linear regression modeling confirmed that the AD effect on greater sSNV burden remains significant (p=0.03) after controlling for potential confounding factors. In addition to AD status, age is also positively correlated with the sSNV burden (p=0.002). FIG. 3D provides a bar graph showing that the significant increase of sSNVs burden in AD brains was only observed for tumor suppressor genes (TSGs) but not for (proto-)oncogenes. FIG. 3E provides a mutation map showing the top 10 recurrently mutated genes in AD and control brains. Asterisks denote the five “hotspot” genes that contain significantly more somatic mutations in AD patients than matched controls (p<0.05). FIG. 3F provides a schematic showing distribution of somatic mutations in two AD hotspot genes, TET2 and ASXL1. The color and height of each lollipop denote the mutation type and the number of carrying individuals. FIG. 3G provides boxplots showing that somatic mutations in AD brains showed significantly higher allele fractions than controls, with a larger increase when only considering TSGs or AD hotspot genes, suggesting the clonal expansion of cells that carry the somatic mutations. The increase of allele fraction was calculated using the ratio of medians between AD and control groups.

FIGS. 4A-4D provide cluster maps, pie charts, bar graphs, and a scatter-plot showing that deleterious somatic mutations are enriched in microglial clones of AD brains. FIG. 4A provides cluster maps and pie charts showing that 10× single-nucleus RNA-seq (snRNAseq) confirms the high purity of microglia in CSF1R⁺ nuclei sorted from AD and control PFC samples. Clustering results suggest about 80% of the sorted nuclei are microglia (dark grey), whereas another 3-9% are CNS-associated macrophages (CAMs, light grey). Minimal blood cell contamination is confirmed with up to 1% monocytes and the absence of B cells, T cells, and red blood cells. OPC, oligodendrocyte progenitor cell. FIG. 4B provides a bar graph showing that the ratios of mutant allele fractions between sorted microglial and neuronal nuclei of the same AD brains, estimated by amplicon sequencing. Ten of the 11 profiled AD somatic mutations demonstrated at least 4× microglial enrichment. FIG. 4C provides bar graphs showing, as examples, that four somatic mutations in CHIP genes show significantly higher allele fractions in microglia than the fractions in the other three populations (p<0.05, two-tailed Wilcoxon test), suggesting their microglial origins. Each nuclei population was sorted four times from each AD brain sample to serve as replicates. Error bar, SE. FIG. 4D provides a scatter-plot showing that all but the FGFR1 mutations are shared between microglia and whole-blood samples of the same individual, indicating a common origin of these somatic mutations.

FIGS. 5A-5F provide graphs showing that somatic CNVs in AD microglia are associated with a pro-inflammatory, disease-related signature. FIG. 5A provides a graph showing that microglia from AD brains contain nominally more CHIP-somatic copy number variants (sCNVs) compared to age-matched controls, even in a small sample (N=31 each). Triangles highlight an individual with multiple CHIP-sCNVs. FIG. 5B provides a graph showing that AD brains show a trend (p=0.06, permutation test) towards a higher fraction of CHIP-sCNV-carrying microglia than age-matched controls. FIG. 5C provides a graph showing odds ratios of CHIP-sCNV-carrying cells between AD and control individuals across different cell types. Microglia-CAM (p=0.06) and microglia (p=0.07) have the smallest nominal p-values in permutation test compared to CAMs (p=0.11), astrocytes (p=0.09), oligodendrocytes (p=0.50), OPC (p=0.40), and ExN (p=0.99). OPC, oligodendrocyte progenitor cell. ExN, excitatory neuron. FIG. 5D provides a volcano plot that shows differentially expressed genes between AD donor microglia-CAMs with and without CHIP-sCNV. Positive fold-change indicates upregulation in microglia-CAMs with CHIP-sCNV. Disease-associated microglia (DAM)-associated upregulated genes are colored red. FIG. 5E provides a graph showing significantly (adjusted p<0.05, hypergeometric test) enriched gene ontology terms for genes upregulated in microglia-CAMs with CHIP-sCNV. FIG. 5F provides a graph showing enrichment of microglial state modules among genes upregulated in microglia-CAMs with CHIP-sCNV. Significant pathways implicate inflammation and the DAM transcriptional state.

FIGS. 6A-6F provide bar graphs, boxplots, pie charts, plots, and a heat map showing the identification and functional annotation of sSNVs in RNA-seq data. FIG. 6A provides bar graphs showing mutation type and tri-nucleotide context of sSNVs. T-to-C(A-to-G) candidates were ignored because they were more likely to be RNA-editing sites widespread in the human genome. FIG. 6B provides boxplots showing similar sequencing depth between the AD and control brain samples in each AD cohort. The overall higher depth in MayoRNAseq may explain the higher base-line mutation burden in control brain samples than ROSMAP. FIG. 6C provides pie charts showing genic annotation and functional impact prediction of sSNVs identified from AD and control brain samples. FIG. 6D provides a plot showing that AD brains had significantly more deleterious sSNVs than controls (p=0.047, linear regression) after controlling for potential confounding factors. FIG. 6E provides a heat map showing that absent expression of blood marker genes in snRNAseq of unsorted ROSMAP brains confirmed minimal blood contamination. FIG. 6F provides a plot showing that the AD increase was consistently significant when the proportion of blood cell types indicated by the expression of marker genes was additionally considered in the linear regression model. RBC, red blood cell.

FIGS. 7A-7G provide graphs, boxplots, bar graphs, pie charts, and a scatter-plot showing benchmarking and validation results of sSNV and sIndels identified from panel sequencing. FIGS. 7A-7B provide graphs and boxplots showing comparable sequencing depth (FIG. 7A) and coverage (FIG. 7B) between AD and control PFC samples, calculated based on the consensus reads after UMI-based read collapsing. FIGS. 7C-7D provide graphs showing detection sensitivity (FIG. 7C) and accuracy of allele fraction estimation (FIG. 7D) for the panel sequencing and somatic mutation identification pipeline, benchmarked by in vitro mixture of the DNA samples of two unrelated individuals with varied genome ratios. FIGS. 7E-7F provide bar graphs and pie charts showing that amplicon sequencing validation confirmed high accuracy for identified sSNV and sIndels in AD and control samples (FIG. 7E). Somatic-I mutations are those with mutant allele fractions at least 3× larger than the fractions of the other two error alleles of the same genomic position, whereas somatic-II are those that were further validated by comparing its mutant allele fractions in a negative control sample (FIG. 7F). FIG. 7G provides a scatter-plot showing mutant allele fraction of validated somatic mutations between panel sequencing (discovery) and amplicon sequencing (validation). Amplicon sequencing was performed using newly extracted DNA from the corresponding brain sample, therefore the allele fractions could be varied between discovery and validation stages.

FIGS. 8A-8E provide bar graphs, pie charts, schematics, and a mutation map showing identification and functional annotation of sSNVs in panel sequencing data. FIG. 8A provides bar graphs showing mutation type and tri-nucleotide context of sSNVs. FIG. 8B provides pie charts showing genic annotation and functional impact prediction of sSNVs identified from AD and control PFC samples. FIG. 8C provides bar graphs showing that the proportion of somatic mutation carriers increases with age. AD patients had a significantly larger proportion of carriers with somatic mutations in AD hotspot genes than matched controls (p=5.6e⁻⁵). FIG. 8D provides schematics showing similar distributions between somatic mutations identified in AD brains and previously reported CHIP mutations in blood. FIG. 8E provides a mutation map showing that genes in the PI3K-PKB/Akt pathway contained significantly more somatic mutations in AD brains (12% of AD samples vs 7% of control samples; p<0.05).

FIGS. 9A-9D provide sorting plots, an expression map, bar graphs, and scatter-plots showing microglia purity and mutant allele fraction of FANS-sorted nuclei population. FIG. 9A provides sorting plots showing selectively isolated microglia from frozen brain tissues using FANS with an antibody targeting epitopes of CSF1R, a gene highly expressed in microglia. FIG. 9B provides an expression map showing marker gene expression profile for 10× single-nucleus RNA-seq of CSF1R⁺ sorted nuclei. Each column represents a single nucleus, clustered by the t-SNE map based on their expression similarity. About 80% of the sorted nuclei are microglia with high expression of CX3CR1, TMEM119, and P2RY12, whereas another 10% are CNS-associated macrophages (CAMs). Markers for blood cell types (HBA1: red blood cell; CD3E: T cell; CCR7: B cell; FCN1: monocyte) confirm the minimal presence of blood cells in sorted nuclei. CNS, central nervous system. AD microglia showed generally reduced expression of CX3CR1 and P2RY12, consistent with previous findings in AD. FIG. 9C provides bar graphs showing mutant allele fractions across different sorted nuclei populations for all the 11 profiled AD somatic mutations. Four mutations are shown in FIG. 4C as examples. In all but the FGFR1 mutation, we observed significantly higher allele fractions in microglia than neurons (NeuN⁺). Each population of nuclei was sorted four times from each AD brain sample to serve as replicates. FIG. 9D provides scatter-plots showing the correlation of mutant allele fractions between blood and three nuclei populations (NeuN⁺, 5 NeuN⁺, and DAPI⁺) sorted from matched brain samples.

FIGS. 10A-10C provide schematics and graphs showing CHIP-sCNV burden analysis in microglia-CAMs and identification of additional microglia-CAMs with scType. FIG. 10A provides a schematic representation of supervised learning framework and quality-control metrics used to detect additional high-quality microglia-CAMs from SEA-AD. FIG. 10B provides a graph showing that scType'd and pre-annotated microglia-CAMs show similar marker gene expression profiles, with specific expression of microglia and CAM marker genes. FIG. 10C provides a graph showing examples of CHIP-sCNV called in two AD individuals, H21.33.017 (chr13p13-31 deletion) and H21.33.010 (chr22 amplification). Normalized median ratio of expression in sCNV-carrying cells versus non-sCNV-carrying cells displayed per chromosomal region, with chromosome size proportional to number of expressed genes in microglia-CAMs from that chromosome.

FIG. 11 provides graphs showing the integrated snRNAseq atlas of microglia-CAMs in AD and healthy controls. UMAP visualization of covariates of interest does not reveal significant clustering by individual ID, nFeature, or nCount, consistent with successful integration across samples. Microglia and CAMs (with high MRC1 expression) separate into distinct clusters.

FIG. 12 provides graphs showing the odds ratio of AD enrichment for somatic mutation with different mutant allele fraction (MAF) cutoffs. When all the 149 genes targeted by the panel sequencing were considered, a consistent trend of AD enrichment was observed even for somatic mutations with 5% or more MAF. In comparison, when only deleterious somatic mutations in CHIP genes were considered, the 5 odds ratio becomes smaller than 1 when MAF is larger than 4%, implying a depletion of high-fraction CHIP mutations in AD. The dashed line represents the odds ratio of 1, and odds ratios larger and smaller than 1 denote the enrichment and depletion of somatic mutation in AD, respectively.

### DETAILED DESCRIPTION OF THE INVENTION

This disclosure provides compositions and methods for characterizing disorders associated with neuroinflammation, including neurodegenerative and neuroinflammatory diseases, such as Alzheimer's disease (AD), and methods of treating such diseases.

As reported in greater detail below, the invention is based, at least in part, on the discovery that there are significantly higher overall burdens of somatic mutations in AD brains compared to age-matched controls. To investigate whether clonal brain somatic mutation is associated with AD, RNA sequencing data from brain samples of two large AD cohorts were analyzed. Gene panel sequencing revealed AD-specific enrichment of somatic mutations in proliferation-related genes, notably multiple genes implicated in blood cells in clonal hematopoiesis of indeterminate potential (CHIP). Pathogenic somatic mutations were especially enriched in microglia of AD brains, and the high proportion of microglia carrying these mutations suggests that some of these mutations drive clonal microglial proliferation, a phenomenon termed “microCHIP.” The association of microCHIP with AD identifies potential new approaches to AD therapy.

Based in part on these findings, it is an insight of the disclosure that the clonal expansion of microglia is activated by somatic mutations and contributes to AD pathogenesis. Accordingly, the disclosure provides for the identification of subjects having an increase in cortical somatic mutations by sequencing DNA or RNA present in a biological sample of the subject (e.g., blood or cell-free DNA in cerebrospinal fluid), thereby identifying subjects that would likely benefit from therapy using therapeutic agents targeting proliferation-related pathways that are useful to inhibit somatic-mutation-activated microglia, thereby treating neuroinflammation associated disease (e.g., neurodegeneration diseases, such as AD and PD). The disclosure further provides compositions and methods for the detection of low prevalence mutations that identify somatic mutations with high sensitivity.

### Neuroinflammation

Neuroinflammation is widely regarded as chronic, as opposed to acute, inflammation of the central nervous system and has been implicated in many diseases, including Alzheimer's disease (AD), Parkinson's disease (PD), Chronic Traumatic Encephalopathy, Pick's disease, Progressive Supranuclear Palsy, Post-COVID encephalopathy, HIV-associated dementia, depression, and others. Not wishing to be bound by theory, it is thought that alleviating neuroinflammation is likely to reduce neuroinflammatory disease (e.g., AD, PD) severity, reduce microglial proliferation and hence reduce the emergence of mutant clones in brain, and improve patient clinical outcomes. Neuroinflammation involves the activation of microglia and astrocytes, release of cytokines and chemokines, production of reactive oxygen species, and oftentimes the infiltration of peripheral leukocytes into the central nervous system (CNS). In its transient form, neuroinflammation is largely protective; however, mounting evidence from clinical and preclinical investigations indicates that chronic or maladaptive neuroinflammation may be a pathological driver of many neurological diseases. Neuroinflammation may be initiated in response to a variety of cues, including infection, traumatic brain injury, toxic metabolites, or autoimmunity.

In the CNS, including the brain and spinal cord, microglia are the resident innate immune cells that are activated in response to these cues. Microglia can actively survey their environment and change their cell morphology significantly in response to neural injury. Acute inflammation in the brain is typically characterized by rapid activation of microglia. During this period, there is no peripheral immune response. Over time, however, chronic inflammation causes the degradation of tissue and of the blood-brain barrier. During this time, microglia generate reactive oxygen species and release signals to recruit peripheral immune cells for an inflammatory response. Astrocytes are glial cells that are the most abundant cells in the brain. They are involved in maintenance and support of neurons and compose a significant component of the blood-brain barrier. After insult to the brain, such as traumatic brain injury, astrocytes may become activated in response to signals released by injured neurons or activated microglia. Once activated, astrocytes may release various factors, e.g., cytokines or chemokines. Activated glial cells also undergo rounds of proliferation and proliferation increases the likelihood of the emergence of spontaneous somatic mutations in dividing cells that have a proliferative advantage due to these mutations. These proliferative mutations also impair the normal function of those glial cells.

Cytokines are a class of proteins that regulates inflammation, cell signaling, and various cell processes such as growth and survival. Chemokines are a subset of cytokines that regulate cell migration, such as attracting immune cells to a site of infection or injury. Various cell types in the brain may produce cytokines and chemokines such as microglia, astrocytes, endothelial cells, and other glial cells. Physiologically, chemokines and cytokines function as neuromodulators that regulate inflammation and development. In the healthy brain, cells secrete cytokines to produce a local inflammatory environment to recruit microglia and clear the infection or injury. However, in neuroinflammation, cells may have sustained release of cytokines and chemokines which may compromise the blood-brain barrier. Peripheral immune cells are called to the site of injury via these cytokines and may now migrate across the compromised blood brain barrier into the brain. Common cytokines produced in response to brain injury include: interleukin-6 (IL-6), which is produced during astrogliosis, and interleukin-1 beta (IL-10) and tumor necrosis factor alpha (TNF-α), which can induce neuronal cytotoxicity. Although the pro-inflammatory cytokines may cause cell death and secondary tissue damage, they are necessary to repair the damaged tissue. For example, TNF-α causes neurotoxicity at early stages of neuroinflammation, but contributes to tissue growth at later stages of inflammation.

### Alzheimer's Disease (AD) Pathogenesis

The importance of microglia in AD pathogenesis has been demonstrated by large-scale genetic association studies which have identified risk variants in a growing list of microglia-related genes (Kunkle, B. W. et al. Nat Genet 51, 414-430 (2019); Jansen, I. E. et al. Nat Genet 51, 404-413 (2019); Hardy, J. & Escott-Price, V. Hum Mol Genet 28, R235-R240 (2019); Bellenguez, C. et al. Nat Genet 54, 412-436 (2022)). As the primary immune cells in the central nervous system (CNS), microglia play critical roles in brain development, injury response, and pathogen defense, modulating cellular responses involved in aging and neurodegeneration as well. Once abnormally reactive in AD, microglia can promote synaptic and neuronal loss and exacerbate tau proteinopathy (Hickman, S. et al. Nat Neurosci 21, 1359-1369 (2018); Bohlen, C. J. et al. Annu Rev Genet 53, 263-288 (2019)). Recent single-cell transcriptomic studies have depicted specific populations of microglia enriched in AD brains of mouse models and human patients, termed disease-associated microglia (DAM) (Chen, Y. & Colonna, M. J Exp Med 218 (2021)). DAM feature reduced expression of homeostatic genes but elevated expression of genes involved in immune response and phagocytosis (Keren-Shaul, H. et al. Cell 169, 1276-1290 e1217 (2017); Silvin, A. et al. Immunity 55, 1448-1465 e1446 (2022)), though whether DAM are beneficial or detrimental to AD remains unsettled Paolicelli, R. C. et al. Neuron 110, 3458-3483 (2022)).

### Somatic Mutations

Somatic mutations accumulate in all cell types that have been studied, both during normal development and during aging. Clonal expansion, driven by somatic mutations in genes regulating cell proliferation, is considered the major cause of cancer, but has also been recently reported in various non-cancer cell types (Kakiuchi, N. & Ogawa, S. Nat Rev Cancer 21, 239-256 (2021)), often in the absence of visible pathology. Clonal expansion of mutant blood cells, called clonal hematopoiesis of indeterminate potential (CHIP), increases in prevalence with age and is associated with increased risk of hematologic malignancies and cardiovascular disease (Genovese, G. et al. N Engl J Med 371, 2477-2487 (2014); Jaiswal, S. et al. N Engl J Med 377, 111-121 (2017)), likely through inflammatory effects of mutant cells on neighboring nonmutant cells (Avagyan, S. et al. Science 374, 768-772 (2021)). A somatic V600E mutation in BRAF, a common cancer-driver mutation, in the microglial lineage has also been causally implicated in degeneration of neurons secondary to mutant microglial activation in both mouse models and humans (Mass, E. et al. Nature 549, 389-393 (2017)). Previous gene panel sequencing of 20 AD brains (Keogh, M. J. et al. Nat Commun 9, 4257 (2018)) and whole exome sequencing of DNA from micro-dissected neuronal nuclei of 52 AD brains (Park, J. S. et al. Nat Commun 10, 3090 (2019)) found no consistent excess of clonal somatic mutations in AD. However, these studies were extremely limited in their ability to detect clonal somatic mutations by small sample sizes, the examination of neuronal DNA only, and low sequence coverage.

### Methods for Characterizing Chronic Neuroinflammation

In certain aspects, this disclosure provides methods for characterizing chronic neuroinflammation. The methods described herein involve characterizing mutations present in a somatic cell derived from the subject and assessing neuroinflammation in the subject. In particular, certain methods of the disclosure involve measuring a somatic mutation burden from one or more genes experimentally identified herein as being linked with an increased somatic mutation burden in a subject with AD. In some embodiments, methods involve measuring a somatic mutation burden from at least a portion of one or more proliferation-related genes. In some embodiments, methods involve measuring a somatic mutation burden by characterizing mutations present in one or more tumor suppressor genes derived from a biological sample of a subject. In some embodiments, methods do not involve measuring a somatic mutation burden of an oncogene.

In some embodiments, the methods involve measuring a somatic mutation burden from one or more genes. In another embodiment, the methods described herein involve detecting a somatic mutation burden, sSNVs, and/or sIndels in any one or more of the following genes or sets of genes: TET2, ASXL1, KMT2D, ATRX and CBL; CX3CR1, TMEM119, and P2RY12; MS4A7 and MRC1; DNMT3A and TET2; DNMT3A (C.1429+1G>A), PPM1D (Leu484fs), TET2 (Val1371Asp), CBL (Leu380Pro), TET2 (Pro1194Ser), TP53 (Arg280Gly), FGFR1 (Arg473Gln); PI3K Pathway genes: PIK3CA, MTOR, PIK3R1, TSC1, AKT3, AKT1, TSC2, STK11, and DEPDC5; CX3CR1, TMEM119, P2RY12; DNMT3A, TET2, ASXL1, KMT2D, ATRX BCR, CBL, TP53, MLH1, and STAT3. In another embodiment, sSNVs or sIndels are detected in EPPK1, PPM1D, TP53, PDGFRA, TET2, ASXL1, MED12, and CHK2. In another embodiment, sIndels are detected in one or more of the following genes or in this set of genes: EPPK1 (2 bp insertion), PPM1D (10 bp deletion) TP53 (1 bp deletion), PDGFRA (4 bp insertion), TET2 (1 bp deletion), ASXL1 (1 bp insertion), MED12 (24 bp deletion), CHEK2 (1 bp deletion), and TET2 (1 bp insertion). In another embodiment, the gene or gene panel comprises one or all of the following: In some embodiments, methods involve measuring a somatic mutation burden from one or more genes listed in Table 1. In some embodiments, methods involve measuring a somatic mutation burden from one or more genes implicated in the Phosphoinositide 3-kinase (PI3K) pathway (e.g., polynucleotides encoding PTEN, mTOR, PI3KCA, PIK3R2, AKT, DEPDC5, TSC1, TSC2, or another component of the PI3K pathway). a splice-site sSNV in DNMT3A (c.1429+1G>A), FGFR1 (p.Arg506Gln), and two deleterious missense sSNVs in TET2 (p.Pro1194Ser and p.Val1371Asp), In embodiments, AD microglia showed reduced expression of CX3CR1 and P2RY12. In embodiments, sSNVs are enriched in microglial cells relative to neurons (e.g., 2×, 3×, 4×, 5×, 6×, 8×, 10×).

The PI3K pathway is a critical signal transduction system linking oncogenes and multiple receptor classes to many essential cellular functions. It is a commonly activated signaling pathway in human cancer. PI3K is downstream of receptor tyrosine kinases (RTKs) and G protein coupled receptors (GPCRs). PI3Ks transduce signals from growth factors and cytokines by generating phospholipids, which activate serine/threonine kinase AKT and other downstream effector pathways. PTEN is a protein phosphatase that acts as a tumor suppressor by antagonizing PI3K. mTOR is a Ser/Thr kinase that is a component of the PI3K pathway that is activated by AKT. mTOR regulates cell proliferation. RTKs, PI3K, AKT and mTOR are all important components of the PI3K pathway.

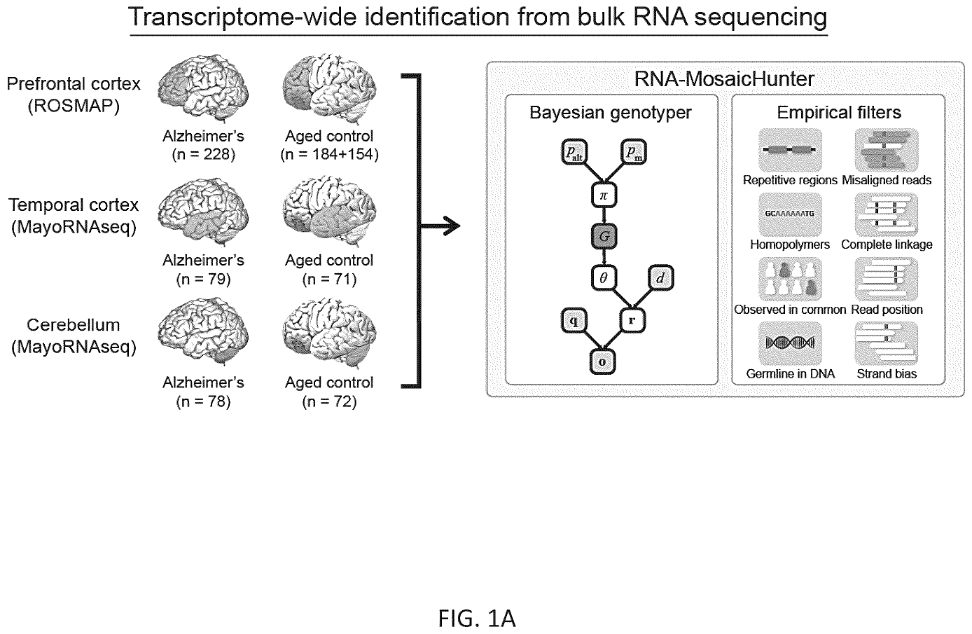

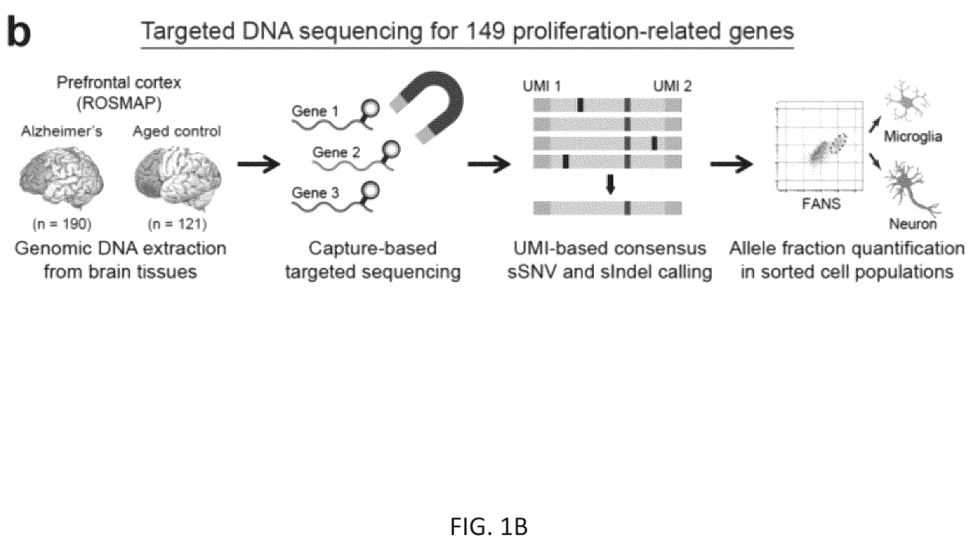

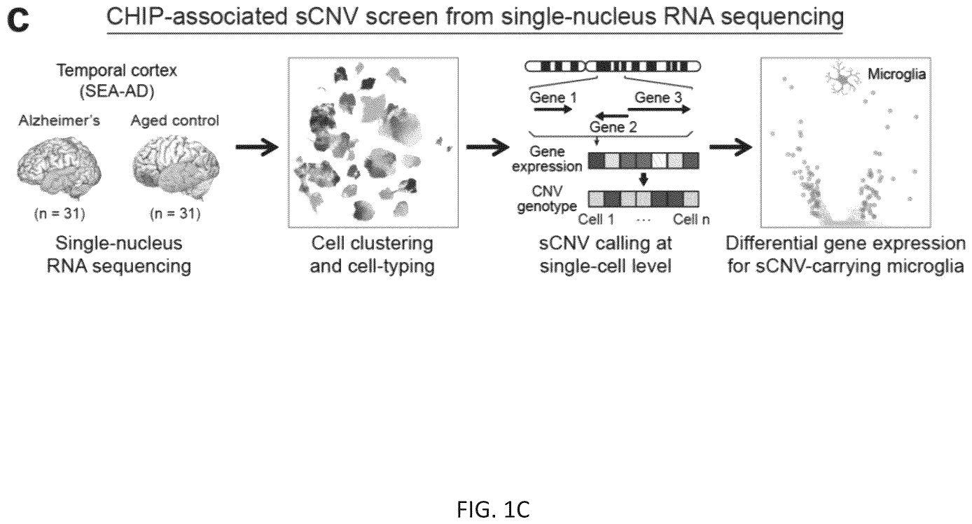

Based on the measured somatic mutation burden, methods of the disclosure involve characterizing chronic neuroinflammation in the subject. In some embodiments, such characterization involves comparing the somatic mutation burden present in a biological sample of the subject to a reference (e.g., the somatic mutation burden present in a biological sample derived from a healthy control subject, or a reference value obtained from prior comparisons). The reference can be a number of somatic mutations identified in the corresponding genes (e.g., proliferation-related genes) of age-matched normal healthy subjects without AD. In some embodiments, a detected increase in the number of somatic mutations in the genes of the subject as compared to the reference is indicative of the subject's having or having a propensity to develop chronic neuroinflammation. The somatic mutations can be detected by any method known in the art. In embodiments, somatic mutations are analyzed using bulk RNA sequencing or deep panel sequencing of polynucleotides derived from a biological sample (e.g., cell free DNA sample derived from blood, serum, CSF, or another liquid sample) or as described in FIG. 1. In some embodiments, an increase in the number of somatic mutations present in a biological sample of a subject (e.g., an increase of 5%, 10%, 15%, 20%, 25%, 30%, 35%, 40%, 45%, 50%, or more) indicates that the subject has or has the propensity to develop chronic neuroinflammation. In some embodiments, an increase by at least 20%, for example, is indicative that the subject has or has an increased propensity to develop chronic neuroinflammation. In some embodiments, the subject is assigned a score based on the detected increase in somatic mutations. For example, a detected increase of about 10% in the number of somatic mutations in a biological sample of a subject relative to a reference is assigned a score of about 1.0, while a detected increase of about 60% may be assigned a risk score of about 6.0. Scores of 2.0 or more are indicative that the subject has or has a propensity to develop chronic neuroinflammation. In other embodiments, the reference can be the number of somatic mutations identified in a biological sample obtained from the subject at an earlier time point, e.g., 6 months, 1 year, 2 years, 5 years, or more, prior to the analysis of a biological sample obtained from the subject at a later time point. Accordingly, in some embodiments, methods involve monitoring the subject over time for an increase in the number of somatic mutations present in a biological sample obtained from the subject.

In some embodiments, methods of measuring a somatic mutation burden involve performing a transcriptome-wide identification of somatic mutations from bulk RNA sequencings. In some embodiments, methods of measuring a somatic mutation burden involve targeted DNA sequencing. In some embodiments, methods of measuring a somatic mutation burden involve bulk RNA sequencing and targeted DNA sequencing, for example, as described in FIG. 1 and Example 1 and Example 2.

### Types of Biological Samples

Compositions and methods described herein involve identifying somatic mutations from nucleic acids taken from a biological sample of a subject. The biological samples are generally derived from the subject in the form of a bodily fluid (e.g., blood, cerebrospinal fluid, phlegm, saliva, sputum, semen, vaginal secretion, or urine) or tissue sample (e.g. a cheek swab, scraping, or tissue sample obtained by biopsy). In some preferred embodiments, the fluid sample is a blood sample or cerebrospinal fluid sample. The cerebrospinal fluid sample can be collected through a procedure called a spinal tap, also known as a lumbar puncture.

### Nucleic Acid Isolation

Once the biological sample is obtained, the sample can be stored or processed. In some embodiments, processing the sample involves isolating nuclei. In some embodiments, nuclei can be prepared from fresh or frozen samples using a chilled nuclear lysis buffer (10 mM Tris-HCl, 0.32 M Sucrose, 3 mM Mg(Acetate)2, 5 mM CaCl₂), 0.1 mM EDTA, pH 8, 1 mM DTT, 0.1% Triton X-100) on ice. Nuclear pellets can be resuspended in ice-cold PBS supplemented with 3 mM MgCl2, filtered, then stained with an anti-NeuN antibody (Millipore MAB377) or a CSF1R antibody. Nuclei can then be sorted by flow cytometry using a custom sheath fluid (1×PBS with 3 mM MgCl2), one nucleus per well into 384- or 96-well plates with each well containing 2.8 μl alkaline nuclear lysis buffer (200 mM KOH, 5 mM EDTA, 40 mM DTT) prechilled on ice. Nuclei can be lysed on ice for 15-30 minutes, and then neutralized on ice in 1.4 μl of neutralization buffer (400 mM HCl, 600 mM Tris-HCl, pH 7.5) allowing for the extraction of nucleic acids.

In one embodiment, bulk DNA is extracted using the QIAamp DNA Mini kit with RNase A treatment. In some embodiments, DNA is extracted from the nuclei of cells sorted based on cell type. The isolation of single nuclei using fluorescence-activated nuclear sorting (FACS) and their whole-genome amplification using multiple displacement amplification (MDA) may be performed as described previously in G. D. Evrony et al., Cell 151, 483-496 (2012); F. B. Dean et al., Genome Res 11, 1095-1099 (2001); G. D. Evrony et al., Neuron 85, 49-59 (2015), all of which are incorporated herein by reference in their entirety. In one embodiment, bulk RNA is isolated using an RNA Extraction Kit, for example, using the RNA extraction kit provided by Qiagen.

### Capture of Target Nucleic Acids

Some embodiments of the disclosure involve capture-based targeted sequencing methods. In particular, in some embodiments, probes are used to selectively bind and capture target nucleic acid molecules of interest for sequencing. In some embodiments, the target nucleic acid molecules of interest encode at least a portion of a proliferation-related gene, for example, any one or more of the genes listed in Table 1. In some embodiments, the target nucleic acids encode at least a portion of a tumor suppressor gene. In some embodiments, the target nucleic acids encode at least a portion of one or more genes selected from the group consisting of those listed in Table 1. In an embodiment, target nucleic acid molecules are captured using nucleic acid probes linked to a capture moiety (affinity tag, antibody, agent, material). In some embodiments, the capture moiety comprises a bead, such as a magnetic bead. The nucleic acid probe can be designed to specifically hybridize to sequences of the target nucleic acid under stringent conditions, particularly under conditions of high stringency, as known in the art. The nucleic acid probe can comprise any number of nucleotides, but preferably comprises at least 10 nucleotides. In some embodiments, following the capture of a target nucleic acid molecule, a UMI is ligated onto an end of a target nucleic acids molecule (e.g., 3′ or 5′ end). In some embodiments, UMIs are ligated onto both the 3′ and 5′ ends of a target nucleic acid molecule.

### Hybrid Capture (HC) Probe Design

The disclosure provides methods to design probe sets to characterize a biological sample comprising a gene of interest (e.g., gene of Table 1, Table 2, or otherwise described herein). In embodiments, the genes are related by function and/or sequence (e.g., proliferation, cell cycle, tumor suppressor). In embodiments, a probe is synthesized and conjugated with a capture molecule, such as biotin, allowing for the low-cost, at-scale enrichment of sequences from biological samples (e.g., samples comprising a neuron, microglial cell, blood cell). Various methods described in U.S. Patent Application Publications 2019/0330706, 2019/019766, and/or 2018/0340215, which are incorporated herein in their entirety, are suitable for use in the methods of the present invention.

The present invention features methods for generating probes for use in characterizing a biological sample. In some embodiments, the design method involves (a) constructing candidate probes targeting gene sequences of interest.

Probes described herein (e.g., hybrid capture probes, a candidate probe or a selected probe) comprise, for example, deoxyribonucleic acid (DNA), ribonucleic acid (RNA), peptide nucleic acid (PNA) and/or other non-naturally occurring nucleic acids.

Methods useful in the invention are described, for example, in Gnirke, et al., Nature biotechnology 27:182-189, 2009, US Patent Publication Nos. 2010/0029498, 2013/0230857, 2014/0200163, 2014/0228223, and 2015/0126377 and International Patent Publication No. WO 2009/099602).

Probes disclosed herein have about or at least about 50%, 55%, 60%, 65%, 70%, 75%, 80%, 85%, 90%, 95%, 99%, or 100% complementarity along a length thereof to gene sequences contained within a target sequence. The length along which sequence identity is measured is at least about 10, 15, 20, 25, 30, 35, 40, 45, 50, 55, 60, 65, 70, 75, 80, 85, 80, 95, 100, 150, 200 nucleotides and/or the full length of a probe.

### Synthesis

The invention features sets of probes (e.g., capture molecules) and methods for producing sets of hybrid capture probes.

In some embodiments, the invention features sets of capture molecules complementary to any gene present in Table 1, Table 2, or otherwise described herein, or to any portion thereof, where non-limiting examples of portions thereof include sequences resulting from 3′ and/or 5′ truncations. In some embodiments, the invention features probes having at least about 30%, 35%, 40%, 45%, 50%, 55%, 60%, 65%, 70%, 75%, 80%, 85%, 90%, 95%, 99%, or 100% nucleotide sequence identity to a polynucleotide described herein. The length along which sequence identity is measured can be 10, 15, 20, 25, 30, 35, 40, 45, 50, 55, 60, 65, 70, 75, 80, 85, 80, 95, 100, 150, 200 nucleotides and/or the full length of a probe.

In embodiments, a set of probes (e.g., capture molecule) contains about or at least about 10, 20, 30, 40, 50, 100, 200, 300, 400, 500, 600, 700, 800, 900, 1000, 10000, 50000, 100000, 200000, 300000, 400000, or more unique probes. In embodiments, each unique probe in the set of probes contains a distinct probe sequence.

Methods for synthesizing a set of hybrid capture probes involve synthesis of oligonucleotides in an array format (e.g., chip). Array synthesis can have the advantages of being customizable and capable of producing long oligonucleotides. TWIST chemistry can also be used to manufacture a set of hybrid capture probes.

In certain embodiments, the probes contain a binding member. In certain example embodiments, the binding member is biotin, a hapten, or an affinity tag. In cases where the hybrid capture probes are biotinylated, the capture probes are captured using a capture molecule (e.g., streptavidin) fixed to a solid support. The capture molecule and/or the binding member can be streptavidin, biotin, a hapten, an affinity tag, an antigen-binding molecule, or an antigen. The hybrid capture probes and/or the solid support can contain more than one distinct binding member or capture molecule, respectively.

The solid support can comprise metal, glass, a polymeric material, or any other suitable material. The support can be planar and/or the support can contain particles or beads. The support can be a biochip. The capture molecule can be coupled to the support by covalent or non-covalent bonds. In some embodiments, hybrid capture probes are directly or indirectly covalently coupled to the solid support.

In other embodiments, the set of probes (e.g., hybrid capture probes) are produced using methods described herein or known to the skilled person. In embodiments, the probes of the present invention include mixed or universal nucleotides, such as inosine or 5-nitroindole (i.e., degeneracy). The mixed or universal base(s) can be included in the bait sequence at the position(s) of a single nucleotide polymorphism (SNP), sSNV, sIndel, or mutation, to optimize the bait sequences to catch both alleles (i.e., mutant and non-mutant). In other embodiments, all known sequence variations (or a subset thereof) can be targeted with multiple probes, rather than by using mixed degenerate probes.

In embodiments, the set of hybrid capture probes are derived from oligonucleotides synthesized in a microarray and cleaved and eluted from the microarray.

In some embodiments, the hybrid capture probes are RNA and/or DNA molecules, as well as derivatives or analogs thereof. In some embodiments the probes are chemically or enzymatically modified or in vitro transcribed RNA molecules including but not limited to those that are more stable and resistant to RNase.

In embodiments, the probes (e.g., hybrid capture probes) comprise about 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides. It can be beneficial in some contexts to use hybrid capture probes having a nucleotide length of no more than about 80, 85, 90, 95, 100, 110, 120, 130, 140, 150, 160, 170, 180, 190, 200, or 250 nucleotides.

In some embodiments, probes generated according to the methods described herein contain non-naturally occurring linkages such as locked nucleic acid (“LNA”) or peptide nucleotide acids.

### Hybrid Capture (HC)

Hybrid capture (HC), also called hybrid selection, relies on specific oligonucleotides (i.e., capture probes or simply “probes”) that selectively hybridize (i.e., bind or capture) to sequences from a gene of interest (e.g., Table 1 or 2).

Hybridization between the polynucleotides and capture probes is conducted under any conditions in which the hybrid capture probes hybridize to target polynucleotides, but do not substantially hybridize to non-target polynucleotides. This can involve selection under high stringency conditions. Following hybridization, the polynucleotide/probe complexes are separated based on the presence of a binding member in each probe, and unbound polynucleotides are removed under appropriate wash conditions that remove the nonspecifically bound polynucleotides, but do not substantially remove polynucleotide probe complexes.

In one embodiment, hybrid capture is carried out using methods including those described herein and those described in Gnirke, et al., Nature biotechnology 27:182-189, 2009, US patent publications No. US 2010/0029498, US 2013/0230857, US 2014/0200163, US 2014/0228223, and US 2015/0126377 and International Patent Publication No. WO 2009/099602, each of which is incorporated by reference in its entirety.

For example, the invention encompasses use of hybrid capture probes of the present invention with the SureSelectXT, SureSelectXT2 and SureSelectQXT Target Enrichment System, the SeqCap EZ kit developed by Roche NimbleGen, a TruSeq® Enrichment Kit developed by Illumina, and other hybridization-based target enrichment methods and kits that add sample-specific sequence tags either before or after the enrichment step.

The hybrid capture methods provided herein can be used for enriching target polynucleotides of interest. The polynucleotides can be related by structure or by function. The polynucleotides can be associated with one or more functions of interest. The target polynucleotides can be enriched by about or at least about 1, 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 95, or 100-fold.

In embodiments, the hybrid capture methods provided herein can be used for enriching for polynucleotides derived from a cell of interest (e.g., microglial cell, neuron, blood cell, or progenitor thereof). Enrichment can involve increasing the concentration of polynucleotides of interest in a sample relative to other polynucleotides in the sample by about or at least about 1, 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 95, or 100-fold.

In embodiments, conditions (e.g., salt concentration and/or temperature) are adjusted such that hybridization between a target sequence and a hybridization probe(s), optionally bound to a solid support, occurs with precise complementary matches or with various degrees of less complementarity depending on the degree of stringency employed. For example, stringent salt concentration can include those containing less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be achieved in the absence of organic solvent, e.g., formamide, while high stringency hybridization can be obtained in the presence of at least about 35% formamide, and most preferably at least about 50% formamide. Stringent temperature conditions can include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

### Characterization

The hybrid capture probes and methods featured in the disclosure can be used for the characterization of a gene or panel of genes (e.g., genes of Table 1 or 2). The hybrid capture probes can be used to characterize a biological sample which may comprise a target sequence(s) or a fragment thereof. The target sequences can be related by structure and/or function. The method may comprise (a) contacting the selected probes to the target sequence or a fragment thereof; and (b) analyzing the target sequence or fragment thereof that hybridizes to one or more of the selected probes.

Analyzing the target sequence or fragment thereof that hybridizes to one or more of the selected probes may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various characterization methods may be used and are described as follows.

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Library preparation may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference). See also Metzker Nature Review Genetics 11, 31-46 (2010).

The sequencing of a polynucleotide can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

Polynucleotides may be characterized and/or enriched by means of a biochip (also known as a microarray) containing hybrid capture probes of the present invention. Biochips generally comprise solid substrates and have a generally planar surface, to which a capture reagent (also called an adsorbent or affinity reagent) is attached. The capture reagent can be a hybrid capture probe(s) or a binding member. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. Such solid supports are suitable for use as solid supports generally in embodiments of the present invention. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Molecular Identifiers

For a convenient detection of polynucleotide/probe complexes, the hybrid capture probes can be coupled to a molecular identifier. Molecular identifiers suitable for use in the present invention include any agent detectable by photochemical, biochemical, spectroscopic, immunochemical, electrical, optical or chemical means. In some embodiments, a probe described herein is linked to a nucleotide sequence that is used for molecular identification.

A wide variety of appropriate molecular identifiers are known in the art, which include fluorescent or chemiluminescent labels, radioactive isotope labels, enzymatic or other ligands. The molecular identifier can be a fluorescent label or an enzyme tag, such as digoxigenin, β-galactosidase, urease, alkaline phosphatase or peroxidase, avidin/biotin complex.

Methods used to detect or quantify the hybridization intensity will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticles including as non-limiting examples quantum dots (Empodocles, et al., Nature 399:126-130, 1999), gold nanoparticles (Reichert et al., Anal. Chem. 72:6025-6029, 2000).

Characterizing a target sequence or fragment thereof that hybridizes to one or more of the hybrid capture probes may be an identifying analysis, wherein hybridization of a selected hybrid capture probe(s) to the target sequence or a fragment thereof indicates the presence of the target sequence within the sample.

### Nucleic Acid Amplification

In some embodiments, nucleic acid molecules obtained from a biological sample derived from a subject are amplified. Nucleic acid amplification involves producing one or more copies of a nucleic acid molecule. An amplification product may be RNA or DNA, and may include a complementary strand to the expressed target sequence. RNA amplification products can be produced initially through reverse transcription (e.g., with reverse transcriptase) to generate cDNA and then optionally from further amplification reactions. The amplification product may include all or a portion of a target sequence, and may optionally be labeled. A variety of amplification methods are suitable for use in the methods described herein, including polymerase-based methods and ligation-based methods. One exemplary amplification technique is the polymerase chain reaction (PCR).

The first cycle of amplification in polymerase-based methods (e.g., PCR) typically involves a primer extension product complementary to the template strand. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR. The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

In some embodiments, a multiple displacement amplification (MDA) reaction is performed to amplify one or more targets of interest. In some embodiments, the targets include proliferation-related genes, including any one or more of the genes listed in Table 1. The MDA reaction can be performed in a 20 μl total reaction volume by addition of an MDA master mix (2 μl 10×Phi29 reaction buffer (Epicentre), 8.4 μl H₂O, 4 μl 10 mM dNTP, 1 μl 1 mM random hexamer (5′ dNdNdNdN*dN*dN-3′ [where*=thiophosophate linkage])(IDT or Thermo-Fisher), 0.4 μl repliPHI polymerase (40U) (Epicentre)). MDA was performed at 30° C. for 16 hours.

### Primers

Primers based on the nucleotide sequences of a target polynucleotide (e.g., polynucleotides encoding at least a portion of a proliferation-related, cell-cycle related, tumor suppressor gene, or a gene of Table 1) may be designed for use in amplification of the target sequences. For use in amplification reactions, such as PCR, a pair of primers is used. The exact composition of the primer sequences is not critical to the invention, but for most applications the primers hybridize to specific sequences under stringent conditions, particularly under conditions of high stringency, as known in the art. The pairs of primers are typically positioned to generate an amplification product. In embodiments, an amplification product comprises at least about 25, 50, 75 or at least about 100 nucleotides. Algorithms for the selection of primer sequences are generally known, and are available in commercial software packages. Primers for use in the methods described herein may be used in standard quantitative or qualitative PCR-based assays to assess transcript expression levels of RNAs defined by a probe set. Alternatively, primers are used in combination with probes, such as molecular beacons in amplifications using real-time PCR. In some embodiments, the primers are designed to hybridize to sequences flanking one or more proliferation-related genes. In some embodiments, the primers are designed to hybridize to sequences flanking one or more tumor suppressor genes. In some embodiments, the primers are designed to hybridize to sequences flanking at least a portion of one or more genes listed in Table 1.

Primers and probes useful in the methods described herein comprise oligonucleotides containing modified backbones or non-natural internucleoside linkages. As is known in the art, a nucleoside is a base-sugar combination and a nucleotide is a nucleoside that also includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Oligonucleotides having modified backbones include those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′ amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Other modifications may be made at other positions on the polynucleotide probes or primers, particularly the 3′ position of the sugar on the 3′ terminal nucleotide or in 2′-5′ linked oligonucleotides and the 5′ position of 5′ terminal nucleotide. Polynucleotide probes or primers may also comprise sugar mimetics, such as cyclobutyl moieties in place of the pentofuranosyl sugar.

Polynucleotide primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. L, ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability.

One skilled in the art recognizes that it is not necessary for all positions in a given polynucleotide probe or primer to be uniformly modified. The present disclosure, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences that are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. In some embodiments, they may provide a UMI. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

### Single Cell Whole Genome Sequencing

Whole genome sequencing (also known as “WGS”) is a process that determines the DNA sequence of an organism's genome. In various embodiments, the genome of a single cell is sequenced, including from a nucleus isolated from the cell. Methods of isolating single cells and nuclei of cells are known in the art. For single cell WGS, whole genome amplification is used to construct a library. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Sequencing of library fragments can be determined by any known method for DNA sequencing. However, high throughput sequencing methods are generally preferred. In one embodiment, the sequencing of a DNA fragment is carried out using commercially available sequencing technology, e.g., SBS (sequencing by synthesis) by Illumina. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing.

All sequencing libraries contain finite pools of distinct DNA fragments. In a sequencing experiment only some of these fragments are sampled. As used herein, the term “coverage” refers to the percentage of genome covered by reads. Coverage also refers to, in shotgun sequencing, the average number of reads representing a given nucleotide in the reconstructed sequence. Biases in sample preparation, sequencing, and genomic alignment and assembly can result in regions of the genome that lack coverage (that is, gaps) and in regions with much higher coverage than theoretically expected. The term depth may also be used to describe how much of the complexity in a sequencing library has been sampled.

### Whole-Genome Sequencing

In some embodiments, DNA libraries are prepared as previously described G. D. Evrony et al., Cell 151, 483-496 (2012); G. D. Evrony et al., Neuron 85, 49-59 (2015), which are incorporated by reference. In some embodiments, 500 ng of amplified DNA from the isolated nuclei described above are sheared on a Covaris E210 focused ultra-sonicator. Paired-end barcoded whole genome sequencing (WGS) libraries can then be prepared with a NEXTflex DNA sequencing kit using 8 cycles of PCR amplification. Paired-end sequencing (100 bp×2 or 101 bp×2) can be performed on Illumina HiSeq 2000 sequencers at the Harvard Biopolymers Facility (Harvard Medical School, Boston MA) and Axeq (Seoul, South Korea).

In some embodiments, identification of somatic mutations involves high average read depth, such that a low frequency mutation is distinguished from an error as the number of correct reads outnumbers any individual errors that may occur, rendering them statistically irrelevant. The sequencing depth typically ranges from 80× to up to thousands, or even millions-fold coverage (e.g., 100, 1,000, 10,000, 20,000, 50,000, 100,000, 250,000, 500,000, 1,000,000, 250,000,000). In some embodiments, targeted DNA sequencing is to a coverage of about or at least about 10×, 20×, 30×, 40×, 50×, 60×, 70×, 80×, 90×, 100×, 200×, 500×, 1000×, 2000×, or more, where a sequencing coverage of 0.01 indicates that a DNA sample has been sequenced such that the amount of DNA sequenced is equivalent in size to about 1% of the corresponding amplicon from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×.

### Whole Transcriptome Sequencing

RNA sequencing (RNA-Seq) is a powerful tool for whole transcriptome profiling. In some embodiments, RNA sequencing is used to identify expressed somatic mutations at the whole-transcriptome scale. RNA sequencing can be performed using methods known in the art. In brief, RNA can be extracted form nucleic prepared as described above. The RNA can be converted into complementary cDNA using a reverse transcriptase enzyme. The cDNA can then be processed for sequencing. To mitigate sequence-dependent bias resulting from amplification complications, a set of unique molecular marker identification sequences can be used to ensure that every cDNA molecule prepared from a nucleic is uniquely labeled. In other embodiments, a molecular barcode is used (see, e.g., Shiroguchi K, et al. Proc Natl Acad Sci USA. 2012 Jan. 24; 109(4):1347-52). After PCR, paired-end deep sequencing can be applied. Rather than counting the number of reads, RNA abundance can be measured based on the number of unique sequences observed for a given cDNA sequence. The barcodes may be optimized to be unambiguously identifiable.

### Compositions and Methods for Assessing Chronic Neuroinflammation

In some aspects, this disclosure provides oligonucleotides that specifically hybridize to a target gene (e.g., tumor suppressor gene, gene of Table 1, gene encoding a component of the PI3 kinase pathway) comprising one or more somatic mutations, wherein the nucleic acid molecule is derived from a subject having or having a propensity to develop chronic neuroinflammation (e.g., AD, PD) as compared to one or more age-matched subjects without neuroinflammation. In some embodiments, the oligonucleotides hybridize to a portion of one or more target genes selected from the group consisting of EPPK1, PPM1D, TP53, PDGFRA, TET2, ASXL1, MED12, and CHK2. In some embodiments, the oligonucleotide hybridizes to a gene at a genomic position that is between 0-100 base pairs upstream of the target gene. In some embodiments, the nucleic acid probe comprises a sequence that hybridizes to the nucleic acid encoding the gene at a genomic position that is between 0-100 base pairs downstream of the target gene. In some embodiments, the oligonucleotides comprise a pair of oligonucleotides useful in the amplification of a target gene. In other embodiments, the oligonucleotides are primers useful for sequencing a target gene. In still other embodiments, the oligonucleotides comprise one or more probes useful for characterizing the presence of absence of an alteration in a target polynucleotide. The primers and probes may comprise RNA, DNA, or a mixture thereof. In some embodiments, the primers and probes comprise at least about 5, 10, 15, 20, 25, 30, 35, 40, 45, 50, 55, 60, 65, 70, 75, 80, 85, 90, 95, or 100 nucleobases. In some embodiments, the primers and probes comprise a length of between 15-25 base pairs. In some embodiments, the primers and probes comprise one or more modified nucleotides. The modified nucleotides may comprise chemistries that prevent degradation or facilitate detection.

In some embodiments, the nucleic acid probes are linked to a capture moiety. The capture moiety can comprise any material or agent (e.g., affinity tag) that allows the probes to be separated from non-target nucleic acids. In some embodiments, the capture moiety is a solid substrate. In some embodiments, the solid substrate is a bead. In some embodiments, the bead is a magnetic bead. In some embodiments, the solid substrate is a chip, e.g., a biochip. In some embodiments, the nucleic acid probes comprise a detectable label. In some embodiments, the detectable label comprises a fluorescent label.

### Compositions and Methods for Regulating Chronic Neuroinflammation

In some aspects, this disclosure provides compositions and methods useful for treating or preventing chronic neuroinflammation (e.g., Alzheimer's disease, and other neurodegenerative conditions), for example, by reducing the level of inflammation in a cell, tissue, or organ of a subject identified as having chronic neuroinflammation according to a method described herein (e.g., FIG. 1, Example 1, 2). Accordingly, certain methods and compositions described herein may be useful for treating disorders associated with chronic neuroinflammation, such as AD or PD, or symptoms thereof.

In one aspect, this disclosure provides a method for inhibiting neuroinflammation. In some embodiments, the method involves using sequence data or the presence of somatic mutations to identify a subject as having or having a propensity to develop chronic neuroinflammation. After identifying a subject as having or having a propensity to develop chronic neuroinflammation, the method further includes contacting a cell, such as a microglial cell, with an effective amount of a therapeutic agent (e.g., any one or more of the agents listed in Table 1).

TABLE 1
Genes and Therapeutic Agents

| Gene | interaction_types | drug_name | drug_claim_name |
|---|---|---|---|
| MTOR | inhibitor | CHEMBL561708 | 9361 |
| PIK3CA | inhibitor | INK-1117 | INK-1117 |
| MTOR | inhibitor | DACTOLISIB | BEZ235 |
| PIK3R1 | inhibitor | RG-7666 | CHEMBL3545324 |
| MTOR | inhibitor | METFORMIN | 9360 |
| AKT1 | allosteric modulator | MK-2206 | 7945 |
| PIK3CA | inhibitor | | 8243 |
| AKT3 | inhibitor | OMIPALISIB | GSK2126458 |
| PIK3CA | inhibitor | AMG-319 | 8917 |
| PIK3CA | inhibitor | BUPARLISIB | BKM120 |
| AKT1 | inhibitor | UPROSERTIB | CHEMBL3137336 |
| PIK3R1 | inhibitor | VS-5584 | CHEMBL1079593 |
| PIK3R1 | inhibitor | GEDATOLISIB | CHEMBL592445 |
| PIK3CA | inhibitor | SF-1126 | CHEMBL2326966 |
| MTOR | inhibitor | GEDATOLISIB | 7940 |
| TP53 | vaccine | | AD.P53-DC |
| PIK3CA | allosteric modulator | | 8968 |
| MTOR | inhibitor | | DCL001086 |
| AKT3 | inhibitor | AZD-5363 | 7709 |
| PIK3CA | inhibitor | AZD-8186 | 8527 |
| PIK3R1 | inhibitor | SF-1126 | CHEMBL2326966 |
| AKT1 | inhibitor | MK-2206 | MK-2206 |
| MTOR | inhibitor | CHEMBL1081312 | 8013 |
| PIK3R1 | inhibitor | DACTOLISIB | DACTOLISIB |
| MTOR | inhibitor | TEMSIROLIMUS | TEMSIROLIMUS |
| PIK3CA | inhibitor | SONOLISIB | PX-866 |
| MTOR | inhibitor | INK-128 | 7933 |
| MTOR | inhibitor | CC-223 | 8914 |
| PIK3CA | inhibitor | TASELISIB | GDC-0032 |
| MTOR | inhibitor | PI-103 | PI-103 |
| MTOR | inhibitor | BENZONATATE | 7699 |
| | | PILARALISIB | |
| PIK3CA | inhibitor | (CHEMBL3360203) | CHEMBL3360203 |
| PIK3CA | inhibitor | PWT-33587 | CHEMBL3545006 |
| PIK3CA | inhibitor | RECILISIB | CHEMBL2219421 |
| TP53 | activator | | DCL000015 |
| PIK3R1 | inhibitor | Puquitinib | CHEMBL3545088 |
| MTOR | inhibitor | VS-5584 | 8382 |
| MTOR | inhibitor | METFORMIN | METFORMIN |
| AKT3 | inhibitor | A-443654 | 8204 |
| MTOR | inhibitor | | 8827 |
| | | BUPARLISIB | BUPARLISIB |
| PIK3R1 | inhibitor | HYDROCHLORIDE | HYDROCHLORIDE |
| MTOR | inhibitor | MLN-0128 | CHEMBL3545097 |
| STAT3 | inhibitor | ACITRETIN | DAP000743 |
| MTOR | inhibitor | PF-04691502 | PF-04691502 |
| PIK3CA | inhibitor | PA-799 | CHEMBL1684984 |
| MTOR | inhibitor | TEMSIROLIMUS | 5892 |
| MTOR | inhibitor | INK-128 | DCL001198 |
| AKT1 | inhibitor | XL-418 | CHEMBL3544935 |
| PIK3CA | inhibitor | TASELISIB | CHEMBL2387080 |
| MTOR | inhibitor | SPARFLOXACIN | 9212 |
| PIK3R1 | inhibitor | PICTILISIB | CHEMBL521851 |
| | | PILARALISIB | |
| PIK3CA | inhibitor | (CHEMBL3218575) | XL147 |
| PIK3CA | inhibitor | VS-5584 | CHEMBL1079593 |
| MTOR | inhibitor | RG-7603 | CHEMBL2331680 |
| PIK3R1 | inhibitor | | PWT33597 |
| MTOR | inhibitor | GEDATOLISIB | CHEMBL592445 |
| AKT3 | inhibitor | GSK-690693 | CHEMBL494089 |
| AKT1 | inhibitor | PERIFOSINE | DCL000194 |
| PIK3CA | inhibitor | COPANLISIB | CHEMBL3218576 |
| AKT1 | inhibitor | AZD-5363 | CHEMBL2178577 |
| PIK3CA | inhibitor | CHEMBL1231533 | 6024 |
| MTOR | inhibitor | APITOLISIB | DCL001189 |
| MTOR | inhibitor | SIROLIMUS | SIROLIMUS |
| MTOR | inhibitor | EVEROLIMUS | EVEROLIMUS |
| MTOR | inhibitor | DS-7423 | CHEMBL3545248 |
| AKT3 | allosteric modulator | | 5921 |
| PIK3CA | inhibitor | PF-04691502 | CHEMBL1234354 |
| PIK3CA | inhibitor | SONOLISIB | CHEMBL411907 |
| PIK3R1 | inhibitor | SF-1126 | SF1126 |
| PIK3CA | inhibitor | PKI-179 | CHEMBL1258517 |
| PIK3R1 | inhibitor | ALPELISIB | BYL719 |
| PIK3CA | inhibitor | APITOLISIB | GDC-0980 |
| AKT3 | allosteric modulator | ARQ-092 | 9429 |
| AKT3 | inhibitor | XL-418 | CHEMBL3544935 |
| AKT1 | inhibitor | MK-2206 | MK2206 |
| MTOR | inhibitor | Panulisib | CHEMBL3545322 |
| PIK3CA | inhibitor | CUDC-907 | CHEMBL3545052 |
| PIK3R1 | inhibitor | GEDATOLISIB | PKI-587 |
| PIK3R1 | inhibitor | Panulisib | CHEMBL3545322 |
| | | BARDOXOLONE | |
| STAT3 | inhibitor | METHYL | DCL000217 |
| PIK3CA | inhibitor | BUPARLISIB | BKM120 |
| PIK3CA | inhibitor | INK-1117 | CHEMBL3545055 |
| PIK3R1 | inhibitor | DACTOLISIB | CHEMBL1879463 |
| PIK3CA | inhibitor | GSK-2636771 | GSK2636771 |
| AKT1 | inhibitor | IPATASERTIB | GDC-0068 |
| MTOR | inhibitor | VOXTALISIB | XL-765 |
| | | TRICIRIBINE | |
| AKT3 | inhibitor | PHOSPHATE | CHEMBL462018 |
| PIK3R1 | inhibitor | SONOLISIB | CHEMBL411907 |
| PIK3CA | inhibitor | LY-3023414 | CHEMBL3544999 |
| AKT1 | inhibitor | IPATASERTIB | DCL001186 |
| AKT1 | allosteric modulator | | 5921 |
| AKT1 | inhibitor | | AS703569 |
| PIK3CA | inhibitor | LY-294002 | 6004 |
| PIK3CA | inhibitor | | 9571 |
| PIK3R1 | inhibitor | SONOLISIB | PX-866 |
| MTOR | inhibitor | SF-1126 | SF1126 |
| PIK3CA | inhibitor | | 8012 |
| AKT1 | inhibitor | AZD-5363 | AZD5363 |
| PIK3CA | inhibitor | DACTOLISIB | CHEMBL1879463 |
| MTOR | inhibitor | RIDAFOROLIMUS | RIDAFOROLIMUS |
| PIK3CA | inhibitor | PA-799 | 7743 |
| MTOR | inhibitor | PA-799 | 7743 |
| MTOR | inhibitor | PI-103 | 5701 |
| MTOR | inhibitor | CC-223 | CHEMBL3545151 |
| PIK3R1 | inhibitor | PF-04691502 | CHEMBL1234354 |
| MTOR | inhibitor | APITOLISIB | GDC-0980 |
| AKT1 | inhibitor | AZD-5363 | 7709 |
| PIK3CA | inhibitor | PI-103 | PI-103 |
| PIK3CA | inhibitor | APITOLISIB | 7888 |
| PIK3CA | inhibitor | RG-7666 | CHEMBL3545324 |
| MTOR | inhibitor | AZD-8055 | AZD8055 |
| MTOR | inhibitor | EVEROLIMUS | EVEROLIMUS |
| AKT3 | inhibitor | EVEROLIMUS | EVEROLIMUS |
| MTOR | inhibitor | | 9571 |
| MTOR | inhibitor | TEMSIROLIMUS | DAP001222 |
| PIK3CA | inhibitor | | 7951 |
| PIK3CA | inhibitor | TASELISIB | 7794 |
| AKT1 | inhibitor | BAY-1125976 | CHEMBL3545049 |
| MTOR | inhibitor | RIDAFOROLIMUS | DCL000624 |
| PIK3CA | inhibitor | GEDATOLISIB | PKI-587 |
| MTOR | inhibitor | INK-128 | MLN0128 |
| MTOR | inhibitor | PF-04691502 | CHEMBL1234354 |
| PIK3CA | inhibitor | ZSTK-474 | CHEMBL586701 |
| PIK3CA | inhibitor | BUPARLISIB | 7878 |
| PIK3CA | inhibitor | DACTOLISIB | BEZ235 |
| | | BGT-226 | |
| PIK3CA | inhibitor | (CHEMBL3545096) | CHEMBL3545096 |
| AKT1 | inhibitor | AFURESERTIB | CHEMBL2219422 |
| AKT1 | inhibitor | MK-2206 | CHEMBL1079175 |
| PIK3CA | inhibitor | | 9424 |
| PIK3R1 | inhibitor | COPANLISIB | BAY80-6946 |
| PIK3CA | inhibitor | | 8383 |
| MTOR | inhibitor | RIDAFOROLIMUS | CHEMBL2103839 |
| AKT3 | inhibitor | MSC-2363318A | CHEMBL3545003 |
| MTOR | inhibitor | Palomid-529 | CHEMBL2141712 |
| AKT1 | inhibitor | MK-2206 | MK2206 |
| MTOR | inhibitor | TEMSIROLIMUS | TEMSIROLIMUS |
| PIK3CA | inhibitor | PICTILISIB | 5682 |
| PIK3CA | inhibitor | ALPELISIB | BYL719 |
| MTOR | inhibitor | LY-3023414 | 8918 |
| BCR | inhibitor | IMATINIB | IMATINIB |
| MTOR | inhibitor | EVEROLIMUS | EVEROLIMUS |
| PIK3R1 | inhibitor | PA-799 | CHEMBL1684984 |
| PIK3CA | inhibitor | CANDICIDIN | 8915 |
| MTOR | inhibitor | AZD-8055 | CHEMBL1801204 |
| MTOR | inhibitor | PKI-179 | CHEMBL1258517 |
| PIK3CA | inhibitor | VOXTALISIB | XL-765 |
| PIK3R1 | inhibitor | COPANLISIB | BAY80-6946 |
| PIK3CA | inhibitor | OMIPALISIB | 8974 |
| PIK3CA | inhibitor | GSK-1059615 | CHEMBL3544966 |
| PIK3CA | inhibitor | AZD-6482 | 8059 |
| PIK3CA | inhibitor | GEDATOLISIB | PKI-587 |
| AKT1 | inhibitor | UPROSERTIB | 7902 |
| PIK3CA | inhibitor | | 8793 |
| AKT1 | inhibitor | XL-418 | DCL000009 |
| AKT1 | inhibitor | OMIPALISIB | GSK2126458 |
| PIK3CA | inhibitor | SF-1126 | SF1126 |
| MTOR | inhibitor | | 8805 |
| AKT3 | inhibitor | MK-2206 | MK2206 |
| MTOR | inhibitor | DACTOLISIB | DCL001085 |
| AKT1 | inhibitor | PERIFOSINE | PERIFOSINE |
| AKT3 | inhibitor | IPATASERTIB | 7887 |
| MTOR | inhibitor | INK-128 | CHEMBL3545056 |
| PIK3CA | inhibitor | APITOLISIB | CHEMBL1922094 |
| PIK3R1 | inhibitor | BUPARLISIB | CHEMBL2017974 |
| PIK3R1 | inhibitor | BUPARLISIB | BKM120 |
| PIK3CA | inhibitor | PI-103 | 5701 |
| PIK3CA | inhibitor | VOXTALISIB | CHEMBL3545366 |
| BCR | inhibitor | DASATINIB | DASATINIB |
| PIK3CA | inhibitor | ZSTK-474 | 7965 |
| PIK3CA | inhibitor | TG100-115 | 5715 |
| AKT3 | inhibitor | AZD-5363 | CHEMBL2178577 |
| PIK3CA | inhibitor | | PWT33597 |
| PIK3R1 | inhibitor | PF-04691502 | PF-4691502 |
| MTOR | inhibitor | DACTOLISIB | 7950 |
| PIK3R1 | inhibitor | OMIPALISIB | GSK2126458 |
| AKT1 | inhibitor | IPATASERTIB | 7887 |
| MTOR | inhibitor | OMIPALISIB | 8974 |
| AKT3 | inhibitor | UPROSERTIB | 7902 |
| MTOR | inhibitor | | BGJ398 |
| STAT3 | antisense | | DPR000181 |
| | | PONATINIB | |
| BCR | inhibitor | HYDROCHLORIDE | CHEMBL2105708 |
| PIK3CA | inhibitor | GEDATOLISIB | 7940 |
| AKT3 | inhibitor | ARQ-092 | CHEMBL3545422 |
| BCR | inhibitor | PONATINIB | PONATINIB |
| STK11 | antibody | LANADELUMAB | 9094 |
| MTOR | inhibitor | TEMSIROLIMUS | TEMSIROLIMUS |
| MTOR | inhibitor | DACTOLISIB | DACTOLISIB |
| AKT1 | inhibitor | MSC-2363318A | CHEMBL3545003 |
| PIK3CA | inhibitor | OMIPALISIB | GSK2126458 |
| MTOR | inhibitor | SIROLIMUS | SIROLIMUS |
| PIK3R1 | inhibitor | DACTOLISIB | BEZ235 |
| | | BGT-226 | |
| MTOR | inhibitor | (CHEMBL3545096) | CHEMBL3545096 |
| MTOR | inhibitor | | 8383 |
| AKT1 | inhibitor | GSK-690693 | 5196 |
| PIK3CA | inhibitor | | BGJ398 |
| MTOR | inhibitor | | 7973 |
| | | PILARALISIB | |
| PIK3CA | inhibitor | (CHEMBL3360203) | 7963 |
| | | PILARALISIB | |
| PIK3R1 | inhibitor | (CHEMBL3218575) | XL147 |
| AKT3 | inhibitor | MK-2206 | CHEMBL1079175 |
| MTOR | inhibitor | VOXTALISIB | XL765 |
| PIK3CA | inhibitor | VS-5584 | 8382 |
| PIK3CA | inhibitor | SONOLISIB | PX-866 |
| AKT1 | inhibitor | AZD-4547 | AZD4547 |
| MTOR | inhibitor | ALPELISIB | BYL719 |
| MTOR | inhibitor | PWT-33587 | CHEMBL3545006 |
| MTOR | inhibitor | DACTOLISIB | CHEMBL1879463 |
| MTOR | inhibitor | | PWT33597 |
| AKT3 | inhibitor | IPATASERTIB | CHEMBL2177390 |
| MTOR | inhibitor | SIROLIMUS | DAP000663 |
| MTOR | inhibitor | | BGT226 |
| AKT3 | inhibitor | | 8181 |
| PIK3CA | inhibitor | CUDC-907 | 8952 |
| AKT3 | inhibitor | AFURESERTIB | 7890 |
| MTOR | inhibitor | DS-3078a | CHEMBL3544963 |
| | inhibitory allosteric | | |
| AKT1 | modulator | MK-2206 | MK2206 |
| AKT1 | inhibitor | | AS703569 |
| AKT1 | inhibitor | | DCL000086 |
| MTOR | inhibitor | RIDAFOROLIMUS | RIDAFOROLIMUS |
| PIK3CA | inhibitor | DS-7423 | CHEMBL3545248 |
| TP53 | inhibitor | BORTEZOMIB | BORTEZOMIB |
| MTOR | inhibitor | VOXTALISIB | CHEMBL3545366 |
| PIK3CA | inhibitor | YOHIMBINE | 8969 |
| PIK3CA | inhibitor | CHEMBL1081312 | 8013 |
| PIK3CA | inhibitor | IDELALISIB | 6741 |
| AKT3 | inhibitor | LY-2780301 | CHEMBL3545134 |
| PIK3R1 | inhibitor | GSK-2636771 | GSK2636771 |
| PIK3CA | inhibitor | CHEMBL1086377 | 8011 |
| MTOR | inhibitor | | 8839 |
| AKT1 | inhibitor | ARQ-092 | CHEMBL3545422 |
| MTOR | inhibitor | GEDATOLISIB | PKI-587 |
| AKT1 | inhibitor | LY-2780301 | CHEMBL3545134 |
| AKT1 | inhibitor | AFURESERTIB | 7890 |
| PIK3R1 | inhibitor | APITOLISIB | GDC-0980 |
| PIK3CA | inhibitor | | 8827 |
| PIK3CA | inhibitor | OMIPALISIB | CHEMBL1236962 |
| | | PILARALISIB | |
| PIK3R1 | inhibitor | (CHEMBL3360203) | CHEMBL3360203 |
| AKT1 | inhibitor | PERIFOSINE | PERIFOSINE |
| PIK3CA | inhibitor | PF-04691502 | PF-4691502 |
| AKT3 | inhibitor | GSK-690693 | 5196 |
| MTOR | inhibitor | | 5704 |
| PIK3CA | inhibitor | PF-04691502 | 7936 |
| MTOR | inhibitor | OSI-027 | OSI-027 |
| AKT3 | inhibitor | IPATASERTIB | GDC-0068 |
| PIK3CA | inhibitor | PICTILISIB | GDC-0941 |
| PIK3CA | inhibitor | AZD-6482 | CHEMBL2165191 |
| STAT3 | inhibitor | ATIPRIMOD | DCL000707 |
| PIK3CA | inhibitor | WORTMANNIN | 6060 |
| MTOR | inhibitor | CC-223 | CC-223 |
| AKT1 | inhibitor | MK-2201 | CHEMBL3545000 |
| AKT3 | inhibitor | MK-2201 | CHEMBL3545000 |
| MTOR | inhibitor | OSI-027 | DCL001095 |
| PIK3CA | inhibitor | | 9425 |
| MTOR | inhibitor | PF-04691502 | PF-4691502 |
| AKT1 | inhibitor | CHEMBL379218 | 5655 |
| MTOR | inhibitor | CC-115 | CHEMBL3545426 |
| PIK3CA | inhibitor | INK-128 | 7933 |
| PIK3CA | inhibitor | COPANLISIB | BAY80-6946 |
| PIK3CA | inhibitor | CHEMBL568150 | 6023 |
| MTOR | inhibitor | LY-3023414 | CHEMBL3544999 |
| PIK3CA | inhibitor | COPANLISIB | BAY80-6946 |
| AKT1 | inhibitor | ENZASTAURIN | DCL000109 |
| PIK3R1 | inhibitor | ALPELISIB | BYL719 |
| MTOR | inhibitor | RIDAFOROLIMUS | DCL001094 |
| MTOR | inhibitor | DACTOLISIB | BEZ 235 |
| PIK3R1 | inhibitor | OMIPALISIB | CHEMBL1236962 |
| PIK3CA | inhibitor | DACTOLISIB | DACTOLISIB |
| MTOR | inhibitor | AZD-8055 | AZD 8055 |
| MTOR | inhibitor | PWT-33579 | CHEMBL3545323 |
| PIK3CA | inhibitor | SONOLISIB | PX-866 |
| PIK3CA | inhibitor | QUERCETIN | SOPHORETIN |
| AKT1 | inhibitor | SR-13668 | CHEMBL3545143 |
| PIK3R1 | inhibitor | QUERCETIN | SOPHORETIN |
| PIK3R1 | inhibitor | LY-3023414 | CHEMBL3544999 |
| MTOR | inhibitor | RIDAFOROLIMUS | DCL000767 |
| MTOR | inhibitor | SF-1126 | CHEMBL2326966 |
| PIK3R1 | inhibitor | APITOLISIB | CHEMBL1922094 |
| AKT1 | inhibitor | GSK-690693 | CHEMBL494089 |
| PIK3R1 | inhibitor | WX-037 | CHEMBL3545385 |
| PIK3CA | inhibitor | DACTOLISIB | BEZ235 |
| BCR | inhibitor | SARACATINIB | SARACATINIB |
| AKT3 | inhibitor | AZD-4547 | AZD4547 |
| PIK3CA | inhibitor | ALPELISIB | 7955 |
| AKT3 | inhibitor | AFURESERTIB | CHEMBL2219422 |
| PIK3R1 | inhibitor | VOXTALISIB | CHEMBL3545366 |
| MTOR | inhibitor | OSI-027 | OSI 027 |
| PIK3R1 | inhibitor | PWT-33587 | CHEMBL3545006 |
| | | TRICIRIBINE | |
| AKT1 | inhibitor | PHOSPHATE | DCL001093 |
| AKT3 | inhibitor | UPROSERTIB | CHEMBL3137336 |
| PIK3R1 | inhibitor | GSK-1059615 | CHEMBL3544966 |
| MTOR | inhibitor | VOXTALISIB | XL765 |
| PIK3CA | inhibitor | BAY-1082439 | CHEMBL3545245 |
| AKT1 | allosteric modulator | ARQ-092 | 9429 |
| AKT1 | inhibitor | IPATASERTIB | GDC-0068 |
| PIK3CA | inhibitor | ALPELISIB | BYL719 |
| PIK3CA | inhibitor | VOXTALISIB | XL765 |
| PIK3R1 | inhibitor | VOXTALISIB | XL-765 |
| | | BGT-226 | |
| PIK3R1 | inhibitor | (CHEMBL3545096) | CHEMBL3545096 |
| MTOR | inhibitor | YOHIMBINE | 8969 |
| PIK3R1 | inhibitor | | BGJ398 |
| AKT3 | allosteric modulator | MK-2206 | 7945 |
| MTOR | inhibitor | AZD-2014 | CHEMBL1078983 |
| MTOR | inhibitor | | DCL000481 |
| PIK3CA | inhibitor | LY-3023414 | 8918 |
| AKT1 | inhibitor | | 8181 |
| PIK3CA | inhibitor | | 8805 |
| AKT1 | inhibitor | NELFINAVIR | NELFINAVIR |
| PIK3CA | inhibitor | WX-037 | CHEMBL3545385 |
| PIK3R1 | inhibitor | DS-7423 | CHEMBL3545248 |
| MTOR | inhibitor | OMIPALISIB | CHEMBL1236962 |
| PIK3CA | inhibitor | | 8978 |
| MTOR | inhibitor | RIDAFOROLIMUS | 7884 |
| PIK3CA | inhibitor | Puquitinib | CHEMBL3545088 |
| PIK3CA | inhibitor | PWT-33579 | CHEMBL3545323 |
| | | BUPARLISIB | BUPARLISIB |
| PIK3CA | inhibitor | HYDROCHLORIDE | HYDROCHLORIDE |
| MTOR | inhibitor | VS-5584 | CHEMBL1079593 |
| PIK3CA | inhibitor | GEDATOLISIB | CHEMBL592445 |
| MTOR | inhibitor | OSI-027 | CHEMBL3120215 |
| PIK3CA | inhibitor | OXAZEPAM | 9563 |
| PIK3R1 | inhibitor | AZD-6482 | CHEMBL2165191 |
| PIK3R1 | inhibitor | SONOLISIB | PX-866 |
| MTOR | inhibitor | TEMSIROLIMUS | TEMSIROLIMUS |
| PIK3CA | inhibitor | BUPARLISIB | CHEMBL2017974 |
| MTOR | inhibitor | EVEROLIMUS | DAP001223 |
| PIK3CA | inhibitor | DACTOLISIB | 7950 |
| PIK3CA | inhibitor | Panulisib | CHEMBL3545322 |
| PIK3CA | inhibitor | SAR-260301 | SAR260301 |
| STAT3 | inhibitor | | OPB 51602 |
| PIK3CA | inhibitor | COPANLISIB | BAY80-6946 |
| MTOR | inhibitor | | DCL000264 |
| MTOR | inhibitor | APITOLISIB | 7888 |
| AKT1 | inhibitor | MK-2206 | DCL000570 |
| DNMT3A | inhibitor | DECITABINE | CHEMBL1201129 |
| AKT1 | inhibitor | A-443654 | 8204 |
| MTOR | inhibitor | DS-3078a | DS-3078A |
| PIK3CA | inhibitor | ALPELISIB | BYL719 |
| MTOR | inhibitor | AZD-8055 | 7714 |
| AKT1 | inhibitor | EVEROLIMUS | EVEROLIMUS |
| PIK3R1 | inhibitor | GEDATOLISIB | PKI-587 |
| PIK3CA | inhibitor | ALPELISIB | CHEMBL2396661 |
| AKT1 | inhibitor | IPATASERTIB | CHEMBL2177390 |
| TP53 | vaccine | | EP-2101 |
| MTOR | inhibitor | GEDATOLISIB | PKI-587 |
| AKT1 | inhibitor | MK-2206 | DCL001092 |
| DNMT3A | inhibitor | AZACITIDINE | CHEMBL1489 |
| | | TRICIRIBINE | |
| AKT1 | inhibitor | PHOSPHATE | CHEMBL462018 |
| PIK3CA | inhibitor | PHENMETRAZINE | 9636 |
| MTOR | inhibitor | APITOLISIB | CHEMBL1922094 |
| PIK3R1 | inhibitor | ZSTK-474 | CHEMBL586701 |
| AKT1 | inhibitor | | DCL000218 |
| PIK3R1 | inhibitor | RECILISIB | CHEMBL2219421 |
| MTOR | inhibitor | DACTOLISIB | BEZ235 |
| MTOR | inhibitor | TORIN1 | 8004 |
| PIK3R1 | inhibitor | PICTILISIB | GDC-0941 |
| PIK3R1 | inhibitor | PI-103 | PI-103 |
| PIK3R1 | inhibitor | COPANLISIB | CHEMBL3218576 |
| PIK3CA | inhibitor | PICTILISIB | CHEMBL521851 |
| MTOR | inhibitor | PF-04691502 | 7936 |
| PIK3R1 | inhibitor | TASELISIB | CHEMBL2387080 |
| MTOR | inhibitor | EVEROLIMUS | 5889 |
| PIK3CA | inhibitor | MLN-1117 | CHEMBL3545379 |
| PIK3CA | inhibitor | DUVELISIB | 7795 |
| MTOR | inhibitor | | 8003 |

In some embodiments, the agent is selected based on the identification of a somatic mutation burden in the gene corresponding to the agent listed in Table 1. In embodiments, the somatic mutation burden is the number of mutations present in the DNA of one or more somatic cells of a subject.

As described herein, the therapeutically effective amount means an amount necessary to provide the indicated therapeutic benefit. As used herein, an effective amount is the amount required to confer a therapeutic effect on the treated patient. Typically, the effective amount is determined based on physical parameters such as age, surface area, weight, height, and condition of the patient. For example, a therapeutically effective amount may be from 0.01 mg to 10 g administered once (q.d.) or twice (b.i.d.) daily. In certain embodiments, the therapeutically effective amount may be administered less than once daily (e.g., every other day, weekly, etc.). In one embodiment, an effective amount is an amount that reduces neuroinflammation within, for example, hours (e.g., 6, 12, 24), days (2, 3, 5, 6 days), weeks (e.g., 1, 2, 3, 4, 5, or 6 weeks), or months (e.g., 1, 2, 3, 4, 5, or 6 months) of administration.

The therapeutic agent can be delivered with a pharmaceutically acceptable carrier, which includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose; starches such as corn starch and potato starch; cellulose and its analogs such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers.

### Hardware and Software

The present invention also relates to a computer system involved in carrying out the methods of the invention relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

The invention provides kits for characterizing a mutation in a target gene present in a somatic cell. For example, a kit may include primers and probes that hybridize to a target gene, and that may be used to characterize somatic single-nucleotide variants (SNVs), and/or measuring somatic mutation burden in a biological sample of a subject. In particular embodiments, kits include one or more reagents for single cell (e.g., microglial cell) isolation, whole genome amplification (e.g., primers), and/or whole genome sequencing. In some embodiments, the kit includes primers for amplifying portions of proliferation-related genes previously identified as having one or more somatic mutations, and or probes that hybridize to the amplified proliferation-related genes. In some embodiments, the kit includes primers for amplifying at least a portion of a gene identified herein as having an increased somatic mutation burden in subjects with AD. In particular, the kit may include primers for amplifying at least a portion of one or more genes selected from those listed in Table 1 or 2. In some embodiments, the kit includes primers for amplifying at least a portion of gene implicated in the PI3K pathway. In some embodiments, the kit includes nucleic acid capture probes for capturing nucleic acids encoding a proliferation-related gene. In some embodiments, the kit includes nucleic acid capture probes for capturing nucleic acids encoding a tumor suppressor gene. In some embodiments, the kit includes a capture probe for capturing one or more genes selected from the genes listed in Table 1. In some embodiments, the kit includes a plurality of capture probes for capturing a panel of genes corresponding to one or more proliferation-related genes, tumor suppressor genes, one or more genes listed in Table 1. In some embodiments, the kit includes reagents for unique molecular identifier (UMI) barcoding. In some embodiments, plurality of capture probes are provided with nucleic acid sequences capable of hybridizing to a gene panel that comprises 149 proliferation related genes. In some embodiments, the gene panel comprises less than 149 proliferation related genes. In some embodiments, the gene panel comprises more than 149 proliferation related genes. In some embodiments, the panel comprises genes encoding polypeptides. In some embodiments, the kit comprises a sterile container containing a reagent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. If desired, the kit is provided together with instructions for identifying somatic single nucleotide variants. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”; “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction” (Mullis, 1994); and “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following methods are put forth so as to provide those of ordinary skill in the art with a complete disclosure and description of how to make and use certain aspects of this disclosure and are not intended to limit the scope of what the inventors regard as their invention.

### EXAMPLES

### Example 1: AD Brains Displayed Increases in Somatic Mutations

Whether brain clonal somatic mutation is associated with Alzheimer's disease was tested by two prospective and orthogonal approaches in >300 Alzheimer's disease samples and >400 control brains (FIG. 1), and consistent increases in overall somatic mutations were found in Alzheimer's disease compared to control, as well as function-specific enrichment in genes previously implicated in clonal hematopoiesis of indeterminate potential (CHIP) and other pre-cancerous conditions.

RNA-MosaicHunter was first developed as a method to identify somatic mutations in 886 bulk RNA sequencing (RNA-seq) data sets of various brain regions including prefrontal cortex (PFC), temporal cortex, and cerebellum, to identify somatic mutations in coding regions of expressed genes (FIG. 1A). The RNA-seq datasets were obtained from two independent harmonized cohorts of aging and dementia, the Rush Religious Orders Study/Memory and Aging Project (ROSMAP) (G. X. Y. Zheng et al., Massively parallel digital transcriptional profiling of single cells. Nature Communications 8, 14049 (2017)) and a collection of brains under the Mayo Clinic Alzheimer's Disease Genetics Studies (MayoRNAseq) (J. Fan et al., Characterizing transcriptional heterogeneity through pathway and gene set overdispersion analysis. Nature Methods 13, 241-244 (2016)), in which the clinical consensus diagnosis of cognitive status was given by expert neurologists based on detailed cognitive and neuropathologic phenotyping. Deep DNA panel sequencing was performed with unique molecular identifiers (UMIs) on 311 prefrontal cortex brain samples from ROSMAP to screen somatic mutations in 149 proliferation-related genes (FIG. 1B), which allowed sensitive detection of somatic mutations with mutant allele fractions (MAFs) as low as 0.1%. With the lists of somatic mutations from both approaches, the mutation burden was compared between Alzheimer's disease and control brains with functional annotation to assess whether somatic mutation was associated with Alzheimer's disease (FIG. 1C). The cell-type identity of somatic mutation-carrying cells was investigated using amplicon sequencing of DNA derived from nuclei isolated through fluorescence-activated nuclei sorting (FANS) (FIG. 1C).

RNA-MosaicHunter was also developed to detect somatic mutations in bulk brain RNA-seq datasets (FIG. 1A). RNA-MosaicHunter first calculated the likelihood of somatic mutation for each genomic position using a Bayesian graphical model, which distinguished true mutations from random sequencing errors by considering the base quality metrics for covered reads. RNA-MosaicHunter also incorporated a series of empirical filters to remove artifacts due to systematic base-calling and alignment errors in RNA-seq. Germline variations were removed by comparing against the matched whole-genome or whole-exome sequencing data of the same individual. Considering the widespread adenosine-to-inosine (A-to-I) RNA editing sites across the genome, where inosine was recognized as guanine (G) and therefore indistinguishable from A-to-G somatic single nucleotide variations (sSNVs) in RNA-seq data, only non-A-to-G sites were considered as sSNV candidates.

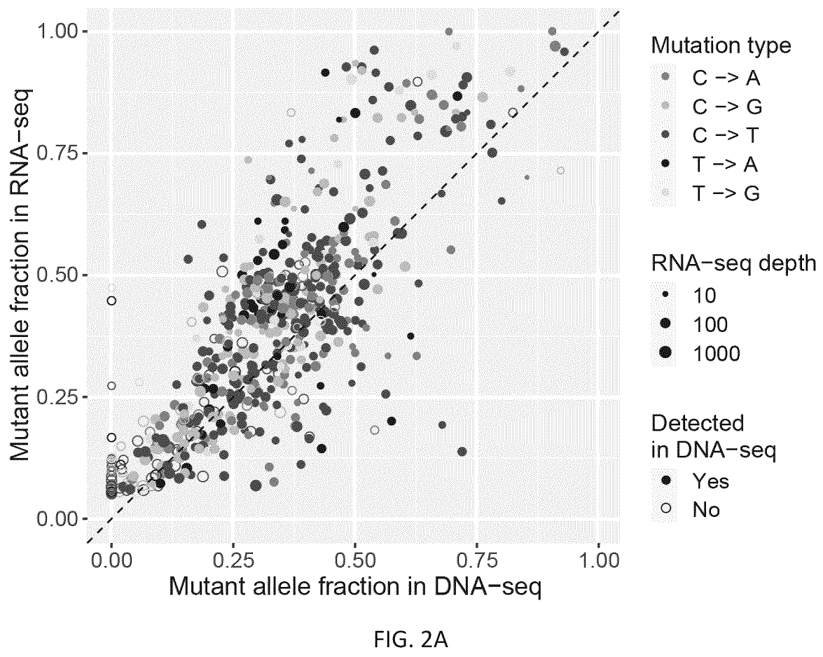

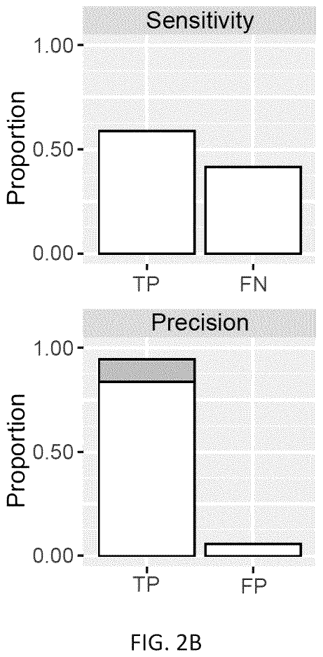

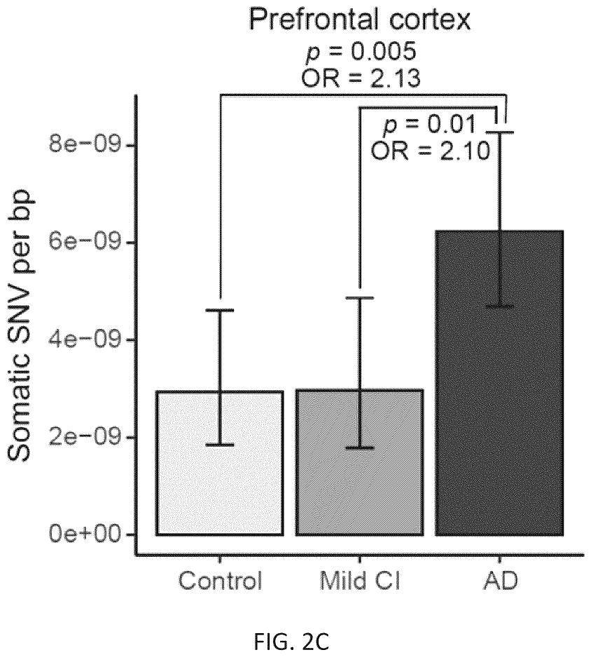

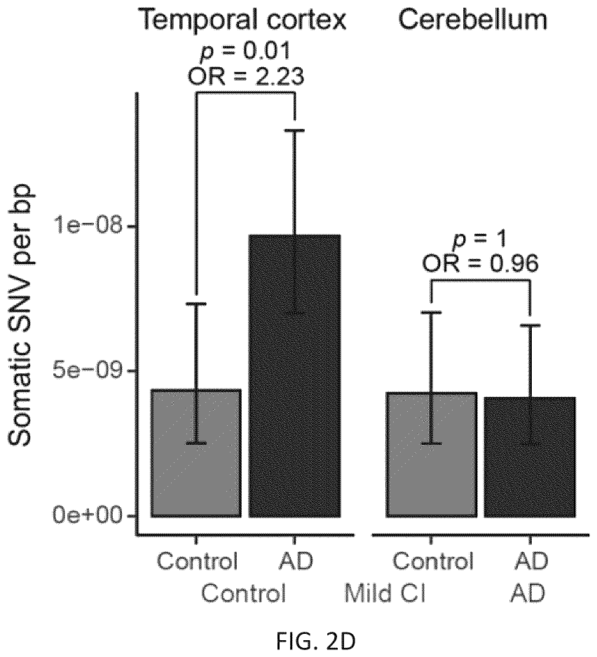

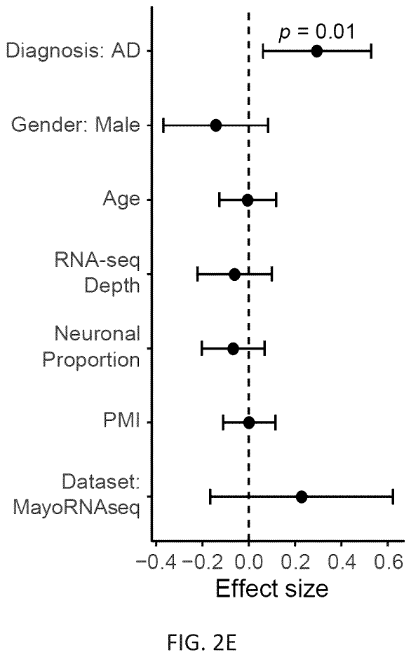

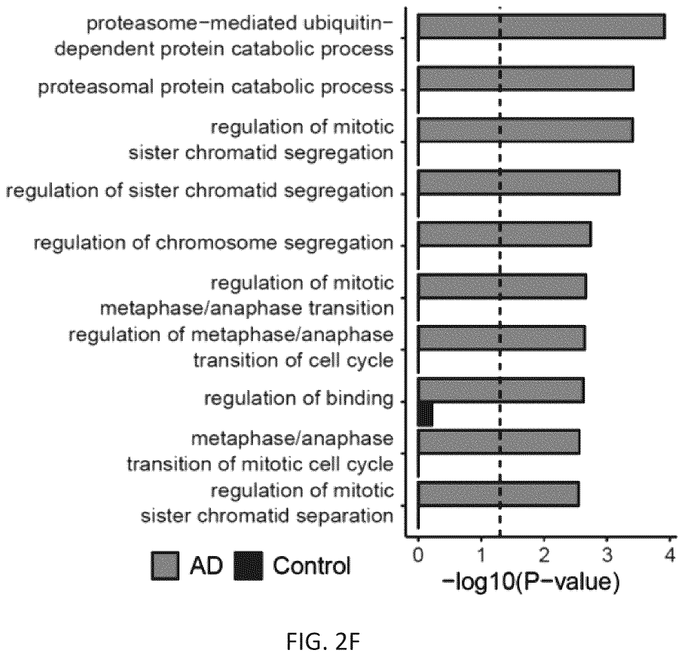

The performance of RNA-MosaicHunter was benchmarked by using 19 esophageal carcinoma samples obtained from The Cancer Genome Atlas (TCGA) Research Network (M. Olah et al., Single cell RNA sequencing of human microglia uncovers a subset associated with Alzheimer's disease. Nat Commun 11, 6129 (2020)). RNA-MosaicHunter identified 613 non-A-to-G sSNVs from the RNA-seq data, and 513 of them were supported by MuTect (J. T. Robinson et al., Integrative genomics viewer. Nat Biotechnol 29, 24-26 (2011)), where calls of the matched whole-exome sequencing data confirmed the accuracy of RNA-MosaicHunter (FIG. 2A). In addition, RNA-MosaicHunter further identified 65 sSNVs with >2% mutant allele fractions in the DNA-seq data, indicating that they were true somatic mutations omitted by MuTect (FIG. 2A). Among 851 MuTect-called exonic mutations with sufficient RNA-seq read coverage, RNA-MosaicHunter successfully recaptured 499 of them (FIG. 2B). In summary, RNA-MosaicHunter achieved 59% sensitivity and 94% precision to identify non-A-to-G sSNVs from the tumor RNA-seq data (FIG. 2B). The sSNVs missed by RNA-MosaicHunter generally had poor coverage or low mutant allele fractions in RNA-seq data, likely due to their low expression level or allele-specific expression in the tumor samples.

### Example 2: A Higher Burden of sSNVs was Observed in Alzheimer's Disease Prefrontal Cortex Samples Relative to Control Subjects

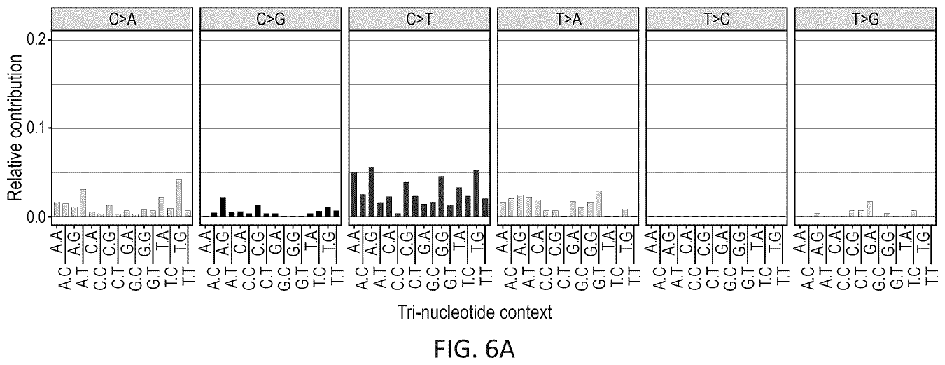

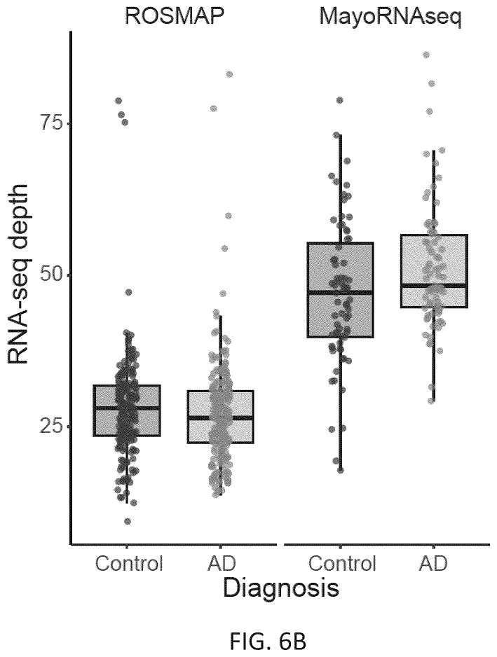

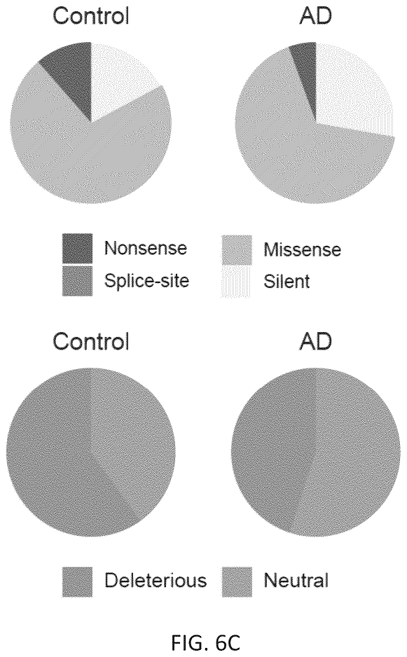

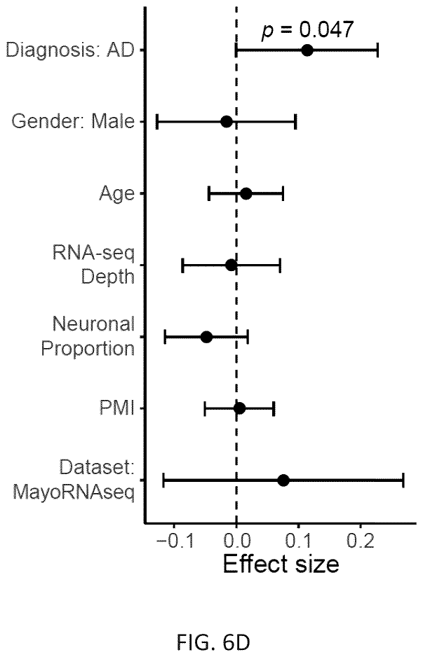

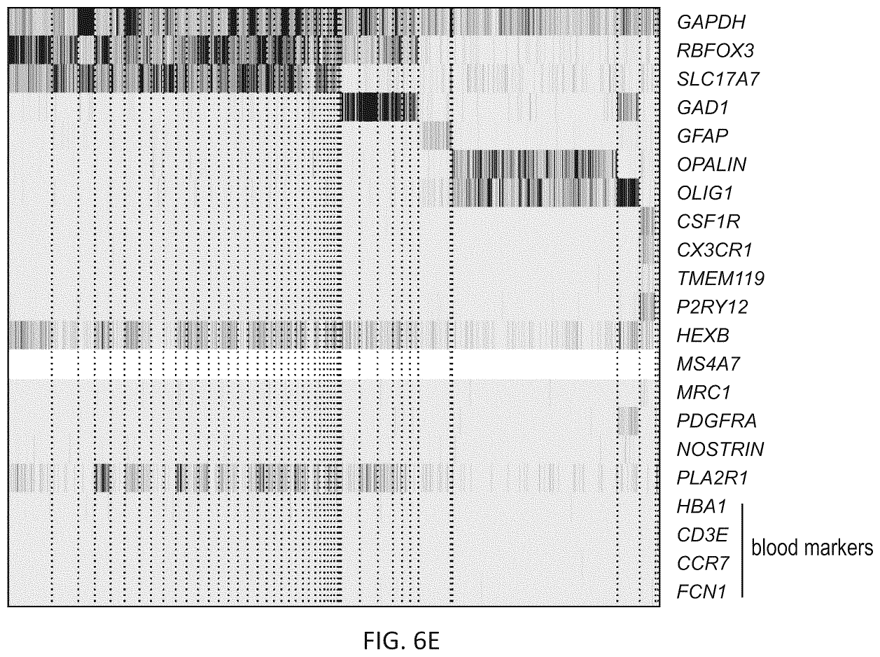

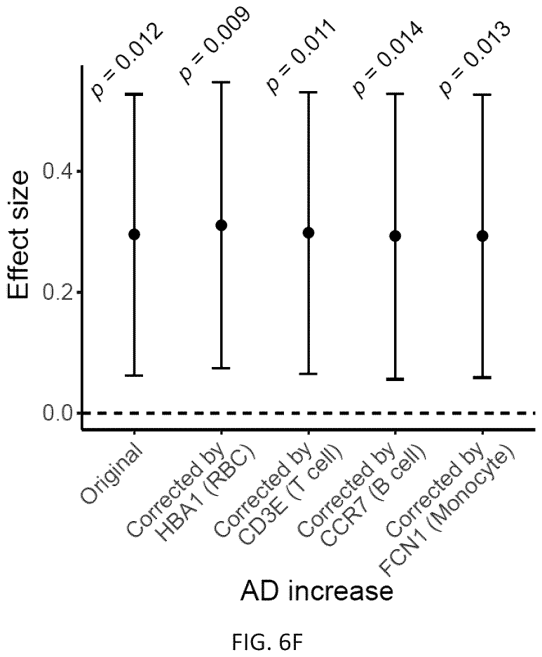

RNA-MosaicHunter was first applied to PFC RNA-seq data of 228 persons with Alzheimer's disease and 338 non-Alzheimer's disease controls (FIG. 6A; Tables 2-3) from the ROSMAP cohort. A significantly higher burden of sSNVs was observed in Alzheimer's disease prefrontal cortex samples compared to controls with a diagnosis of no or only mild cognitive impairment (FIG. 2C; p<0.01; OR=2.1). This finding was tested in a second independent RNA-seq dataset from the MayoRNAseq project, consisting of 300 brain samples from the temporal cortex and cerebellum of 92 patients who died with neuropathologically confirmed Alzheimer's disease and 82 matched controls (FIG. 6A; Tables 2-3). Alzheimer's disease temporal cortex samples showed a consistent increase of sSNV burden compared to neurotypical controls (FIG. 2D; p=0.01; OR=2.2), with a remarkably similar odds ratio to that seen in the ROSMAP prefrontal cortex samples. Interestingly, the disease-specific enrichment of sSNV was limited to the temporal cortex samples and was not observed in cerebellar samples (FIG. 2D; p=1), a brain region not severely affected in Alzheimer's disease. The observed greater sSNV burden in Alzheimer's disease remained significant after controlling for potential confounding factors including gender, age, RNA-seq coverage, neuronal proportion, and batch effect (FIG. 2E and FIG. 6B; p=0.01). This enrichment persisted even when only the subset of sSNVs predicted to have deleterious impact on protein function were considered (FIGS. 6C-6D; p=0.047).

TABLE 2

| | Brain | | # of | | Age | PMI | RNA-seq | Neuronal |
|---|---|---|---|---|---|---|---|---|
| Dataset | region | Diagnosis | samples | Female % | (yr) | (hr) | depth | proportion |
| ROSMAP | Prefrontal | Healthy | 228 | 62.84% | 84.80 ± | 7.37 ± | 28.13 ± | 0.63 ± 0.09 |
| | cortex | control | | | 5.31 | 4.84 | 8.91 | |
| | | Mild cognitive | 154 | 62.34% | 86.93 ± | 7.83 ± | 28.35 ± | 0.60 ± 0.09 |
| | | impairment | | | 4.13 | 5.23 | 8.88 | |
| | | Alzheimer's | 184 | 68.89% | 88.03 ± | 7.14+ | 27.19 ± | 0.58 ± 0.09 |
| | | disease | | | 3.54 | 4.29 | 8.61 | |
| MayoRNAseq | Temporal | Healthy | 71 | 47.89% | 82.85 ± | 6.45 ± | 47.08 ± | 0.54 ± 0.13 |
| | cortex | control | | | 8.26 | 7.07 | 11.96 | |
| | | Alzheimer's | 79 | 60.76% | 82.53 ± | 6.74 ± | 51.20 ± | 0.46 ± 0.09 |
| | | disease | | | 7.67 | 5.43 | 10.21 | |
| | Cerebellum | Healthy | 72 | 47.22% | 82.49 ± | 6.41 ± | 52.96 ± | 0.71 ± 0.11 |
| | | control | | | 8.33 | 7.13 | 12.35 | |
| | | Alzheimer's | 78 | 60.26% | 82.45 ± | 7.62 ± | 61.31 ± | 0.71 ± 0.08 |
| | | disease | | | 7.72 | 7.35 | 12.18 | |

TABLE 3A
(ROSMAP)

| Chr | Pos | Ref | Alt | Gene | Type | Impact | ID |
|---|---|---|---|---|---|---|---|
| 1 | 21560082 | C | T | ECE1 | nonsynonymous | deleterious | 636_120524 |
| 1 | 40214638 | G | A | PPIE | nonsynonymous | deleterious | 119_120418 |
| 1 | 67895888 | C | T | SERBP1 | synonymous | neutral | 202_120424 |
| 1 | 110168313 | C | T | AMPD2 | synonymous | neutral | 884 130923 |
| 1 | 144930627 | A | T | PDE4DIP | nonsynonymous | neutral | 887_130923 |
| 1 | 220284259 | G | A | IARS2 | nonsynonymous | neutral | 571_120521 |
| 2 | 25360357 | G | A | EFR3B | nonsynonymous | deleterious | 03_120405 |
| 2 | 27607766 | G | A | PPM1G | nonsynonymous | neutral | 03 120405 |
| 2 | 54893142 | C | T | SPTBN1 | synonymous | neutral | 119_120418 |
| 2 | 102955409 | C | T | IL1RL1 | synonymous | neutral | 884_130923 |
| 2 | 136513175 | A | T | UBXN4 | nonsynonymous | neutral | 929 131031 |
| 2 | 182542683 | T | A | NEUROD1 | nonsynonymous | deleterious | 202_120424 |
| 3 | 10354407 | C | A | SEC13 | nonsynonymous | deleterious | 571_120521 |
| 3 | 18457586 | G | C | SATB1 | nonsynonymous | deleterious | 119_120418 |
| 3 | 50679747 | C | A | MAPKAPK3 | nonsynonymous | deleterious | 314 120501 |
| 3 | 176744205 | T | G | TBL1XR1 | nonsynonymous | deleterious | 492_120515 |
| 3 | 185213014 | C | T | TMEM41A | synonymous | neutral | 153_120423 |
| 4 | 5841317 | C | T | CRMP1 | synonymous | neutral | 410_120503 |
| 4 | 6086677 | C | T | JAKMIP1 | nonsynonymous | deleterious | 468_120514 |
| 4 | 55161303 | C | T | PDGFRA | nonsynonymous | deleterious | 284_120430 |
| 5 | 36995788 | C | A | NIPBL | synonymous | neutral | 884_130923 |
| 5 | 136448220 | G | A | SPOCK1 | synonymous | neutral | 923 131031 |
| 5 | 177631873 | C | T | HNRNPAB | nonsynonymous | deleterious | 183_120424 |
| 6 | 2766445 | G | A | WRNIP1 | nonsynonymous | deleterious | 380_120503 |
| 6 | 2766445 | G | A | WRNIP1 | nonsynonymous | deleterious | 707_120531 |
| 6 | 72975684 | C | T | RIMS1 | synonymous | neutral | 119 120418 |
| 6 | 127775100 | G | T | KIAA0408 | nonsynonymous | deleterious | 162_120423 |
| 7 | 22194191 | G | A | RAPGEF5 | synonymous | neutral | 119_120418 |
| 7 | 98647273 | C | G | SMURF1 | nonsynonymous | deleterious | 202_120424 |
| 7 | 150937588 | C | T | SMARCD3 | synonymous | neutral | 03_120405 |
| 8 | 42698195 | C | T | THAP1 | nonsynonymous | neutral | 923_131031 |
| 8 | 107531250 | C | T | OXR1 | stopgain | deleterious | 177_120424 |
| 8 | 107531250 | C | T | OXR1 | stopgain | deleterious | 431_120507 |
| 8 | 145537708 | C | T | HSF1 | nonsynonymous | neutral | 699 120605 |
| 8 | 145617483 | G | A | ADCK5 | nonsynonymous | neutral | 366_120502 |
| 8 | 145617483 | G | A | ADCK5 | nonsynonymous | neutral | 884 130923 |
| 9 | 74489626 | G | A | ABHD17B | nonsynonymous | deleterious | 410_120503 |
| 9 | 130986602 | G | A | DNM1 | unknown | neutral | 119_120418 |
| 9 | 139944785 | C | G | ENTPD2 | nonsynonymous | neutral | 72_120417 |
| 10 | 1061710 | G | A | GTPBP4 | synonymous | neutral | 56 120417 |
| 10 | 27313439 | G | A | ANKRD26 | nonsynonymous | deleterious | 12 120410 |
| 10 | 27313439 | G | A | ANKRD26 | nonsynonymous | deleterious | 679_120604 |
| 10 | 104163181 | G | A | PSD | nonsynonymous | deleterious | 111_120418 |
| 11 | 608769 | G | A | PHRF1 | nonsynonymous | deleterious | 270 120430 |
| 11 | 47187883 | T | G | ARFGAP2 | nonsynonymous | deleterious | 03_120405 |
| 11 | 65653039 | G | A | FIBP | nonsynonymous | deleterious | 03_120405 |
| 12 | 6230460 | G | T | VWF | synonymous | neutral | 707 120531 |
| 12 | 7015712 | C | T | LRRC23 | stopgain | deleterious | 202 120424 |
| 12 | 7023107 | C | G | LRRC23 | nonsynonymous | neutral | 202_120424 |
| 12 | 50513969 | C | T | COX14 | nonsynonymous | deleterious | 177_120424 |
| 12 | 51497482 | G | A | TFCP2 | nonsynonymous | deleterious | 923_131031 |
| 12 | 64825579 | C | T | XPOT | synonymous | neutral | 202_120424 |
| 12 | 118599665 | G | A | TAOK3 | synonymous | deleterious | 03_120405 |
| 12 | 123780561 | A | C | SBNO1 | nonsynonymous | neutral | 571_120521 |
| 13 | 67800999 | G | A | PCDH9 | nonsynonymous | deleterious | 665_120530 |
| 13 | 76429410 | A | C | LMO7 | nonsynonymous | deleterious | 787_130530 |
| 14 | 91681811 | G | A | C14orf159 | nonsynonymous | neutral | 236_120425 |
| 14 | 91681811 | G | A | C14orf159 | nonsynonymous | neutral | 884_130923 |
| 15 | 48435124 | C | A | MYEF2 | nonsynonymous | deleterious | 196_120424 |
| 15 | 59063826 | C | A | FAM63B | nonsynonymous | neutral | 571_120521 |
| 15 | 69076803 | C | T | ANP32A | synonymous | neutral | 03_120405 |
| 16 | 624614 | C | T | PIGQ | synonymous | neutral | 202_120424 |
| 16 | 2229182 | G | A | CASKIN1 | nonsynonymous | neutra | 270_120430 |
| 16 | 19726353 | A | T | KNOP1 | nonsynonymous | deleterious | 929_131031 |
| 16 | 30960674 | G | C | ORAI3 | nonsynonymous | neutral | 119_120418 |
| 17 | 1442212 | A | T | PITPNA | nonsynonymous | deleterious | 202_120424 |
| 17 | 48625767 | C | G | SPATA20 | nonsynonymous | neutral | 929_131031 |
| 17 | 61824248 | G | A | CCDC47 | nonsynonymous | deleterious | 549_120517 |
| 17 | 61824248 | G | A | CCDC47 | nonsynonymous | deleterious | 929_131031 |
| 17 | 65955937 | C | G | BPTF | nonsynonymous | neutral | 929_131031 |
| 17 | 73753083 | G | C | ITGB4 | nonsynonymous | deleterious | 787_130530 |
| 17 | 73900910 | C | T | MRPL38 | stopgain | deleterious | 653_120529 |
| 17 | 76136972 | G | A | TMC8 | nonsynonymous | neutral | 410_120503 |
| 17 | 79954599 | G | A | ASPSCR1 | synonymous | neutral | 884_130923 |
| 18 | 12725520 | C | A | PSMG2 | nonsynonymous | neutral | 929_131031 |
| 19 | 541473 | A | C | CDC34 | nonsynonymous | neutral | 202_120424 |
| 19 | 19770511 | C | T | ATP13A1 | synonymous | neutral | 884_130923 |
| 19 | 41893501 | C | G | EXOSC5 | nonsynonymous | deleterious | 410_120503 |
| 19 | 55750856 | G | C | PPP6R1 | nonsynonymous | deleterious | 929_131031 |
| 20 | 1373630 | C | T | FKBP1A | nonsynonymous | deleterious | 884_130923 |
| 20 | 48565848 | G | A | RNF114 | nonsynonymous | neutral | 108_120418 |
| 20 | 60968609 | T | G | CABLES2 | nonsynonymous | deleterious | 884_130923 |
| 21 | 47611838 | C | G | LSS | nonsynonymous | deleterious | 492_120515 |
| 22 | 17619220 | G | A | CECR5 | synonymous | neutral | 571_120521 |
| 22 | 19435000 | G | C | C22orf39 | nonsynonymous | deleterious | 410_120503 |
| 22 | 22058150 | G | A | YPEL1 | nonsynonymous | deleterious | 202_120424 |
| 22 | 42300950 | G | A | SREBF2 | synonymous | neutral | 428_120507 |
| 22 | 42300950 | G | A | SREBF2 | synonymous | neutral | 791_130530 |
| 22 | 47158687 | G | A | TBC1D22A | nonsynonymous | deleterious | 25_120411 |

TABLE 3B
(MayoRNAseq)

| Chr | Pos | Ref | Alt | Symbol | Type | Impact | ID |
|---|---|---|---|---|---|---|---|
| 1 | 14105452 | C | T | PRDM2 | nonsynonymous | deleterious | 1010_TCX |
| 1 | 14105452 | C | T | PRDM2 | nonsynonymous | deleterious | 1956_CER |
| 1 | 15888779 | A | T | DNAJC16 | nonsynonymous | neutral | 11495_TCX |
| 1 | 44422107 | G | A | IPO13 | nonsynonymous | deleterious | 11263_CER |
| 1 | 45821075 | T | A | TESK2 | nonsynonymous | neutral | 1214_TCX |
| 1 | 45821075 | T | A | TESK2 | nonsynonymous | neutral | 834_CER |
| 1 | 75199071 | C | T | TYW3 | nonsynonymous | deleterious | 1034_TCX |
| 1 | 75199071 | C | T | TYW3 | nonsynonymous | deleterious | 129_TCX |
| 1 | 145579249 | C | T | PIAS3 | nonsynonymous | deleterious | 11494_TCX |
| 1 | 150471047 | A | T | TARS2 | synonymous | neutral | 937_CER |
| 1 | 161726630 | C | T | DUSP12 | stopgain | deleterious | 1963_CER |
| 1 | 161726630 | C | T | DUSP12 | stopgain | deleterious | 1963_TCX |
| 1 | 206628281 | G | T | SRGAP2 | unknown | neutral | 1952_CER |
| 1 | 206628281 | G | T | SRGAP2 | unknown | neutral | 886_TCX |
| 1 | 222802081 | A | T | MIA3 | nonsynonymous | neutral | 11279_CER |
| 1 | 223989982 | C | A | TP53BP2 | nonsynonymous | deleterious | 11353_TCX |
| 1 | 228401183 | G | A | OBSCN | nonsynonymous | deleterious | 11267_CER |
| 1 | 228401183 | G | A | OBSCN | nonsynonymous | deleterious | 11267_TCX |
| 1 | 237995919 | G | A | RYR2 | nonsynonymous | deleterious | 11404_CER |
| 1 | 237995919 | G | A | RYR2 | nonsynonymous | deleterious | 11404_TCX |
| 2 | 37217890 | G | A | HEATR5B | synonymous | neutral | 1302_TCX |
| 2 | 39033733 | C | A | DHX57 | nonsynonymous | deleterious | 11331_TCX |
| 2 | 39033733 | C | A | DHX57 | nonsynonymous | deleterious | 1919_CER |
| 2 | 39893389 | C | T | TMEM178A | nonsynonymous | neutral | 11499_TCX |
| 2 | 55844287 | C | A | SMEK2 | nonsynonymous | deleterious | 6894_TCX |
| 2 | 71633283 | G | T | ZNF638 | nonsynonymous | deleterious | 11295_TCX |
| 2 | 71633283 | G | T | ZNF638 | nonsynonymous | deleterious | 11499_TCX |
| 2 | 96952544 | C | T | SNRNP200 | synonymous | neutral | 812_CER |
| 2 | 96952544 | C | T | SNRNP200 | synonymous | neutral | 812_TCX |
| 2 | 101670616 | G | A | TBC1D8 | synonymous | neutral | 11342_CER |
| 2 | 109086344 | A | T | GCC2 | nonsynonymous | neutral | 11263_CER |
| 2 | 109086344 | A | T | GCC2 | nonsynonymous | neutral | 11303_TCX |
| 2 | 128262995 | C | A | IWS1 | stopgain | deleterious | 11472_TCX |
| 2 | 128262995 | C | A | IWS1 | stopgain | deleterious | 11505_TCX |
| 2 | 136872607 | C | A | CXCR4 | synonymous | neutral | 11370_TCX |
| 2 | 176860339 | T | A | KIAA1715 | nonsynonymous | neutral | 1052_CER |
| 2 | 197157356 | T | A | HECW2 | nonsynonymous | deleterious | 1085_TCX |
| 2 | 202260139 | C | A | TRAK2 | nonsynonymous | neutral | 1960_TCX |
| 3 | 36873423 | T | A | TRANK1 | nonsynonymous | neutral | 11374_TCX |
| 3 | 56763404 | T | A | ARHGEF3 | nonsynonymous | neutral | 1937_TCX |
| 3 | 69134282 | C | T | ARL6IP5 | synonymous | neutral | 1935_CER |
| 3 | 99568674 | T | A | FILIP1L | nonsynonymous | neutral | 135_TCX |
| 3 | 133320211 | G | A | TOPBP1 | synonymous | neutral | 7016_CER |
| 3 | 148765848 | C | A | HLTF | nonsynonymous | deleterious | 11279_TCX |
| 3 | 148765848 | C | A | HLTF | nonsynonymous | deleterious | 11463_TCX |
| 3 | 194373563 | C | A | LSG1 | nonsynonymous | neutral | 11481_CER |
| 3 | 194373563 | C | A | LSG1 | nonsynonymous | neutral | 946_TCX |
| 4 | 10079430 | C | A | WDR1 | nonsynonymous | deleterious | 871_TCX |
| 5 | 6606977 | T | A | NSUN2 | synonymous | neutral | 11506_CER |
| 5 | 37125400 | C | T | C5orf42 | synonymous | neutral | 11397_TCX |
| 5 | 54579061 | T | A | DHX29 | nonsynonymous | deleterious | 11371_CER |
| 5 | 54579061 | T | A | DHX29 | nonsynonymous | deleterious | 1919_CER |
| 5 | 132039283 | C | A | KIF3A | nonsynonymous | neutral | 11505_TCX |
| 5 | 139908652 | C | A | ANKHD1, | nonsynonymous | neutral | 1943_TCX |
| | | | | EIF4EBP3 | | | |
| 5 | 140957898 | T | A | ANKHD1-DIAPH1 | nonsynonymous | deleterious | 11305_TCX |
| 5 | 140957898 | T | A | DIAPH1 | nonsynonymous | deleterious | 11371_TCX |
| 5 | 142779303 | G | A | NR3C1 | stopgain | deleterious | 11275_CER |
| 5 | 178045766 | C | A | CLK4 | nonsynonymous | neutral | 1123_TCX |
| 6 | 43014822 | G | A | CUL7 | synonymous | neutral | 11303_TCX |
| 6 | 88344628 | A | T | ORC3 | nonsynonymous | neutral | 11370_TCX |
| 6 | 99997415 | G | A | CCNC | synonymous | neutral | 1271_TCX |
| 6 | 99997415 | G | A | CCNC | synonymous | neutral | 1925_TCX |
| 6 | 117996933 | A | C | NUS1 | nonsynonymous | neutral | 1089_CER |
| 6 | 117996933 | A | C | NUS1 | nonsynonymous | neutral | 1089_TCX |
| 6 | 151779689 | A | T | ARMT1 | nonsynonymous | neutral | 937_CER |
| 7 | 21469502 | C | T | SP4 | nonsynonymous | deleterious | 931_CER |
| 7 | 27570902 | C | A | HIBADH | nonsynonymous | neutral | 11377_TCX |
| 7 | 38803116 | G | A | VPS41 | nonsynonymous | neutral | 6870_TCX |
| 7 | 65445218 | G | A | GUSB | nonsynonymous | deleterious | 11323_CER |
| 7 | 80456792 | T | G | SEMA3C | synonymous | neutral | 11425_CER |
| 7 | 80456792 | T | G | SEMA3C | synonymous | neutral | 11425_TCX |
| 7 | 100033087 | G | A | PPP1R35 | nonsynonymous | neutral | 1936_TCX |
| 7 | 100175357 | C | A | LRCH4 | nonsynonymous | neutral | 1010_TCX |
| 7 | 100175357 | C | A | LRCH4 | nonsynonymous | neutral | 886_TCX |
| 8 | 38099844 | G | T | DDHD2 | synonymous | neutral | 11507_CER |
| 8 | 38099844 | G | T | DDHD2 | synonymous | neutral | 813_CER |
| 8 | 62430102 | C | A | ASPH | nonsynonymous | deleterious | 11505_TCX |
| 8 | 97318716 | C | T | PTDSS1 | synonymous | neutral | 11358_TCX |
| 8 | 99044506 | C | T | MATN2 | nonsynonymous | neutral | 11492_TCX |
| 8 | 99044506 | C | T | MATN2 | nonsynonymous | neutral | 933_TCX |
| 9 | 123330657 | T | A | CDK5RAP2 | nonsynonymous | neutral | 851_CER |
| 9 | 125719450 | A | T | RABGAP1 | nonsynonymous | neutral | 11357_TCX |
| 10 | 863743 | T | A | LARP4B | synonymous | neutral | 787_CER |
| 10 | 120892078 | A | T | FAM45A | nonsynonymous | deleterious | 886_TCX |
| 10 | 135020368 | G | C | KNDC1 | nonsynonymous | deleterious | 991_CER |
| 10 | 135020368 | G | C | KNDC1 | nonsynonymous | deleterious | 991_TCX |
| 11 | 4139645 | G | A | RRM1 | nonsynonymous | deleterious | 11495_TCX |
| 11 | 8016050 | C | A | EIF3F | nonsynonymous | deleterious | 11468_CER |
| 11 | 8016050 | C | A | EIF3F | nonsynonymous | deleterious | 11468_TCX |
| 11 | 61545875 | G | T | MYRF | stopgain | deleterious | 871_TCX |
| 11 | 67925594 | G | A | SUV420H1 | nonsynonymous | deleterious | 1944_CER |
| 12 | 7303584 | C | A | CLSTN3 | nonsynonymous | neutral | 7015_CER |
| 12 | 7303584 | C | A | CLSTN3 | nonsynonymous | neutral | 7015_TCX |
| 12 | 53454318 | G | T | TNS2 | nonsynonymous | deleterious | 11267_TCX |
| 12 | 53454318 | G | T | TNS2 | nonsynonymous | deleterious | 871_TCX |
| 12 | 62949836 | T | A | MON2 | synonymous | neutral | 11266_CER |
| 12 | 62949836 | T | A | MON2 | synonymous | neutral | 11266_TCX |
| 12 | 62949836 | T | A | MON2 | synonymous | neutral | 736_TCX |
| 12 | 112888139 | C | T | PTPN11 | nonsynonymous | deleterious | 6952_TCX |
| 12 | 132490672 | C | T | EP400 | nonsynonymous | neutral | 11249_CER |
| 12 | 132490672 | C | T | EP400 | nonsynonymous | neutral | 1159_CER |
| 13 | 29002012 | T | A | FLT1 | nonsynonymous | neutral | 11349_CER |
| 13 | 29002012 | T | A | FLT1 | nonsynonymous | neutral | 11349_TCX |
| 13 | 29002012 | T | A | FLT1 | nonsynonymous | neutral | 812_CER |
| 13 | 49064340 | T | A | RCBTB2 | synonymous | neutral | 791_TCX |
| 13 | 49064340 | T | A | RCBTB2 | synonymous | neutral | 886_TCX |
| 13 | 115051802 | G | T | UPF3A | nonsynonymous | deleterious | 1105_TCX |
| 14 | 59806877 | A | T | DAAM1 | nonsynonymous | deleterious | 11377_TCX |
| 14 | 59806877 | A | T | DAAM1 | nonsynonymous | deleterious | 11452_CER |
| 15 | 25953160 | C | A | ATP10A | nonsynonymous | deleterious | 11293_TCX |
| 15 | 42132404 | C | G | JMJD7-PLA2G4B, | nonsynonymous | deleterious | 948_CER |
| | | | | PLA2G4B | | | |
| 15 | 42132404 | C | G | JMJD7-PLA2G4B, | nonsynonymous | deleterious | 948_TCX |
| | | | | PLA2G4B | | | |
| 15 | 44865849 | C | A | SPG11 | nonsynonymous | deleterious | 746_TCX |
| 15 | 44865849 | C | A | SPG11 | nonsynonymous | deleterious | 966_CER |
| 15 | 64418368 | C | T | SNX1 | nonsynonymous | neutral | 6870_CER |
| 15 | 91493736 | G | T | UNC45A | stopgain | deleterious | 11472_TCX |
| 16 | 1877786 | G | T | FAHD1 | nonsynonymous | deleterious | 777_TCX |
| 16 | 15715730 | T | A | KIAA0430 | nonsynonymous | deleterious | 791_TCX |
| 16 | 30666433 | G | T | PRR14 | nonsynonymous | deleterious | 962_TCX |
| 16 | 31228280 | T | A | PYDC1 | stopgain | deleterious | 11317_CER |
| 16 | 88643618 | G | T | ZC3H18 | synonymous | neutral | 1929_CER |
| 16 | 88643618 | G | T | ZC3H18 | synonymous | neutral | 871_TCX |
| 16 | 89352548 | T | A | ANKRD11 | nonsynonymous | deleterious | 1951_TCX |
| 17 | 1579275 | C | A | PRPF8 | stopgain | deleterious | 1047_TCX |
| 17 | 1964827 | G | A | SMG6 | nonsynonymous | deleterious | 11306_CER |
| 17 | 1964827 | G | A | SMG6 | nonsynonymous | deleterious | 11306_TCX |
| 17 | 56389950 | G | A | BZRAP1 | synonymous | neutral | 834_TCX |
| 17 | 66914279 | T | A | ABCA8 | nonsynonymous | deleterious | 11486_TCX |
| 17 | 73043669 | G | A | KCTD2 | synonymous | neutral | 1214_TCX |
| 18 | 9570363 | G | A | PPP4R1 | synonymous | neutral | 1089_TCX |
| 18 | 29848030 | G | A | GAREM | nonsynonymous | deleterious | 1058_CER |
| 18 | 54354126 | A | T | WDR7 | nonsynonymous | neutral | 11495_TCX |
| 18 | 54354126 | A | T | WDR7 | nonsynonymous | neutral | 7015_TCX |
| 18 | 60646406 | G | A | PHLPP1 | synonymous | neutral | 849_CER |
| 19 | 5144310 | C | T | KDM4B | nonsynonymous | deleterious | 1926_CER |
| 19 | 5144310 | C | T | KDM4B | nonsynonymous | deleterious | 1926_TCX |
| 19 | 5144310 | C | T | KDM4B | nonsynonymous | deleterious | 6914_CER |
| 19 | 7618785 | A | T | PNPLA6 | nonsynonymous | neutral | 1923_TCX |
| 19 | 11032065 | G | T | CARM1 | nonsynonymous | deleterious | 886_TCX |
| 19 | 15349778 | C | A | BRD4 | stopgain | deleterious | 129_TCX |
| 19 | 57966802 | G | A | VN1R1 | synonymous | neutral | 11255_TCX |
| 19 | 57966802 | G | A | VN1R1 | synonymous | neutral | 11357_CER |
| 20 | 3649629 | G | A | ADAM33 | nonsynonymous | neutral | 1015_TCX |
| 20 | 3649629 | G | A | ADAM33 | nonsynonymous | neutral | 1123_TCX |
| 20 | 31413753 | C | T | MAPRE1 | nonsynonymous | neutral | 11460_CER |
| 21 | 30329177 | T | A | LTN1 | nonsynonymous | deleterious | 11443_TCX |
| 21 | 30329177 | T | A | LTN1 | nonsynonymous | deleterious | 834_CER |
| 21 | 34074285 | A | C | SYNJ1 | nonsynonymous | deleterious | 11433_TCX |
| 21 | 34632954 | A | T | IFNAR2 | synonymous | neutral | 991_TCX |
| 22 | 36696975 | T | A | MYH9 | synonymous | neutral | 11307_TCX |
| 22 | 38043281 | C | G | SH3BP1 | synonymous | neutral | 11344_CER |
| 22 | 38043281 | C | G | SH3BP1 | synonymous | neutral | 11344_TCX |
| 22 | 47287230 | A | T | TBC1D22A | synonymous | neutral | 11456_TCX |

To ensure that the larger number of somatic mutations in Alzheimer's disease brains did not reflect contamination by blood, the presence of blood cell types was measured by analyzing gene markers for blood cells in both bulk and single-nucleus RNA-seq data of ROSMAP and/or MayoRNAseq. Blood contamination, as measured by blood-related transcripts in these brain samples, was confirmed to be minimal (FIG. 6E); correcting the data for any minimal blood did not change the elevated burden of somatic mutation in Alzheimer's disease brains (FIG. 6F). The results from these two RNA-seq datasets consistently indicated that clonal somatic mutations in the cerebral cortex are increased in Alzheimer's disease patients.

Using Gene Ontology annotation, sSNVs were found in Alzheimer's disease brains to be significantly enriched in genes related to ubiquitin-dependent proteolysis, as well as in genes that regulate cell cycle and proliferation, and this enrichment pattern was not found in sSNVs identified in control brains (FIG. 2F). Considering the role of proliferation-related genes in amplifying somatic mutations, the results indicated that somatic mutations in proliferation-related genes may be more common in Alzheimer's disease cerebral cortex.

### Example 3: AD Brains Harbored More sSNVs Among Proliferation-Related Genes than Controls

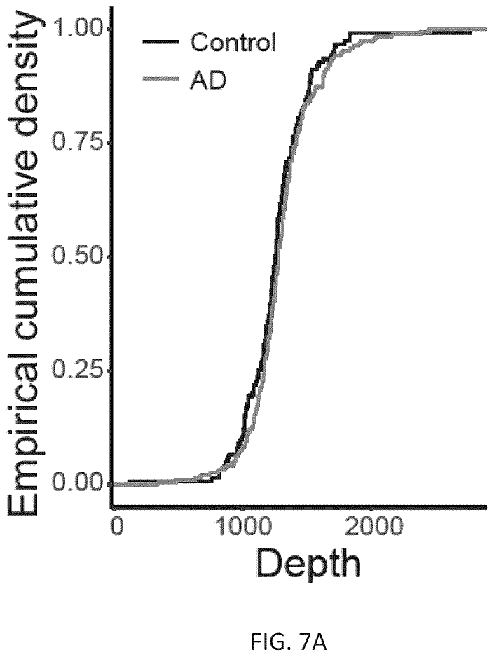

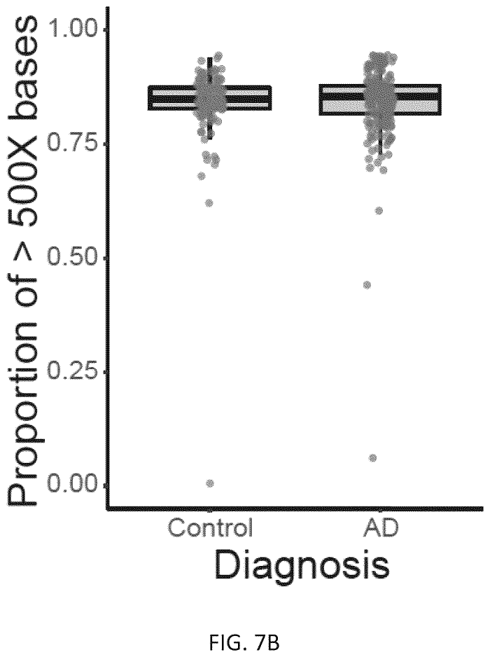

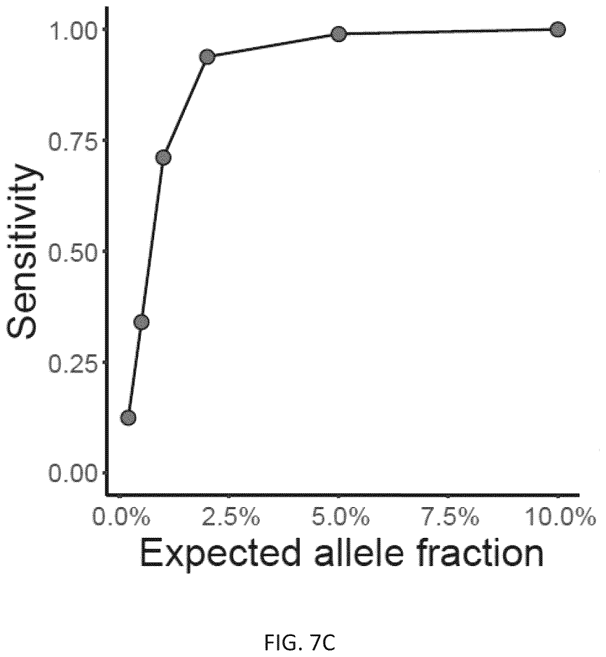

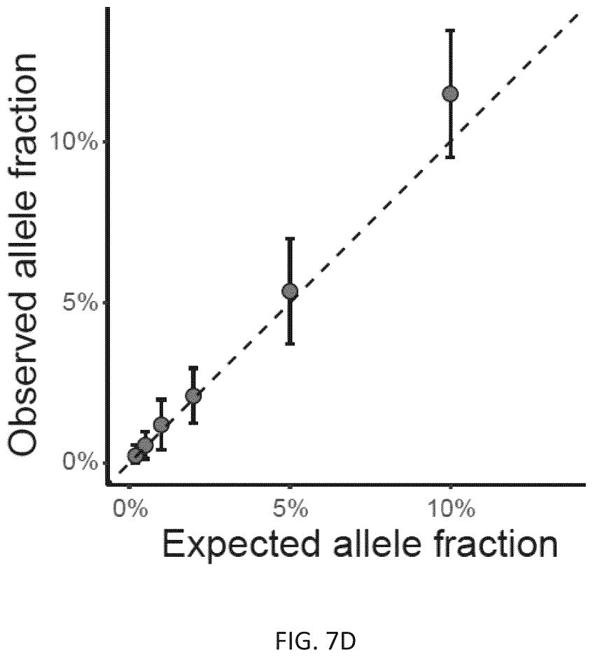

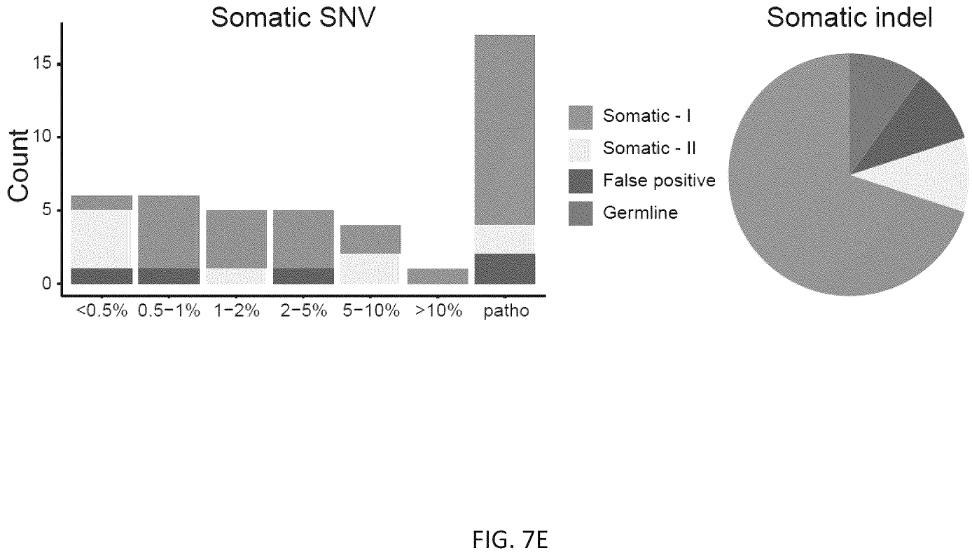

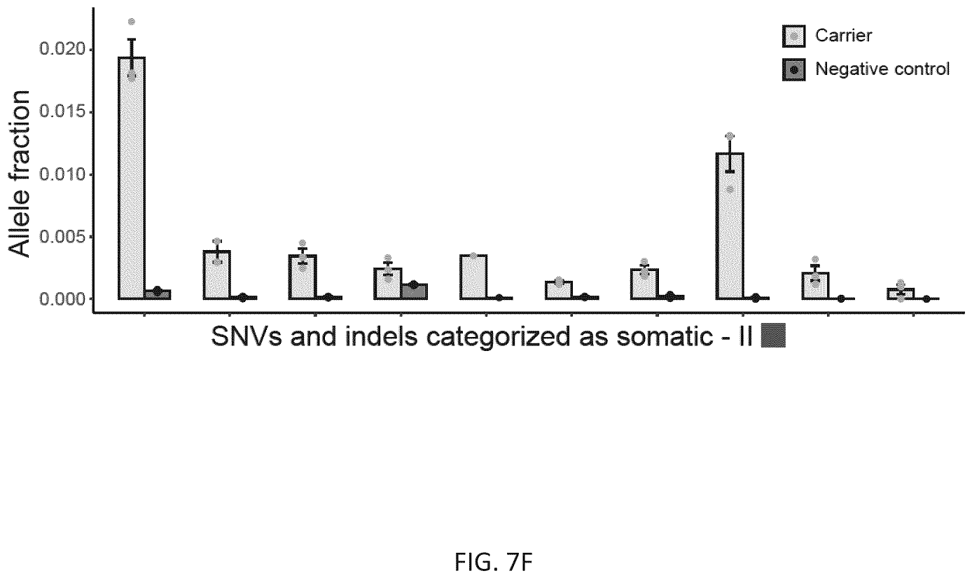

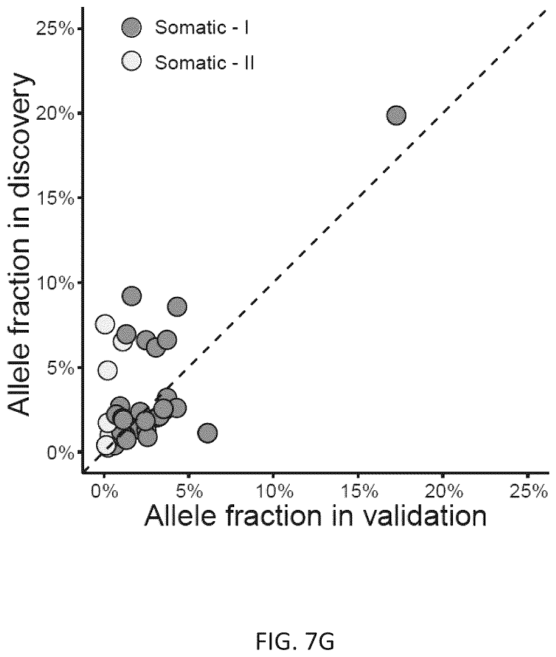

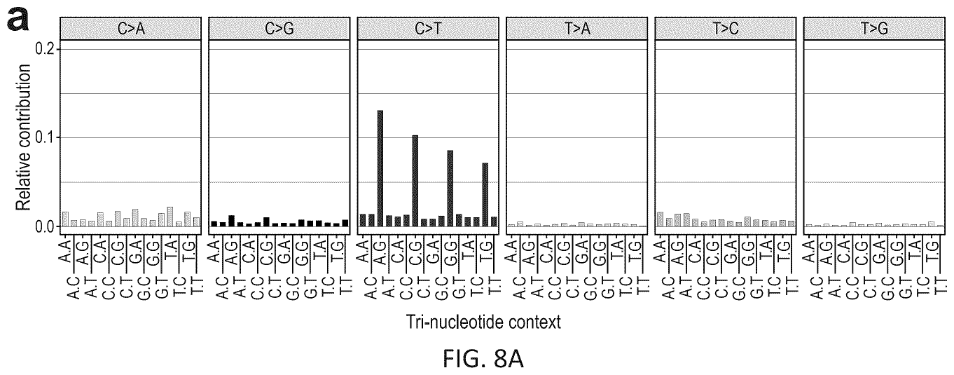

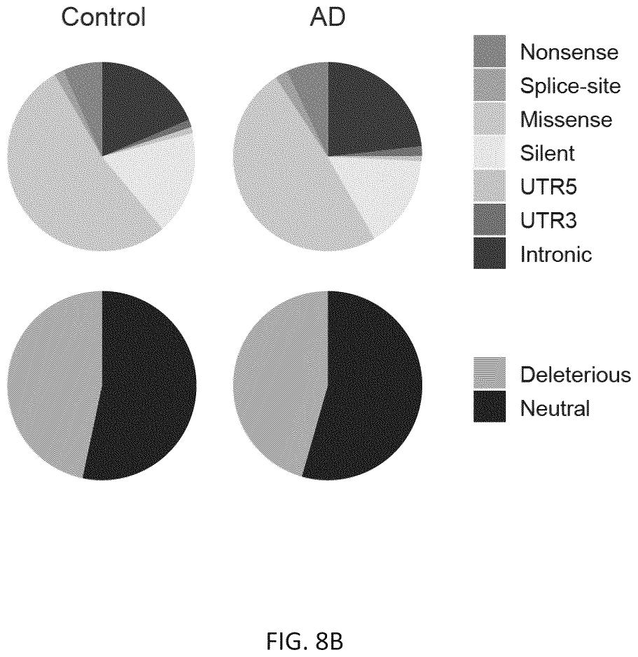

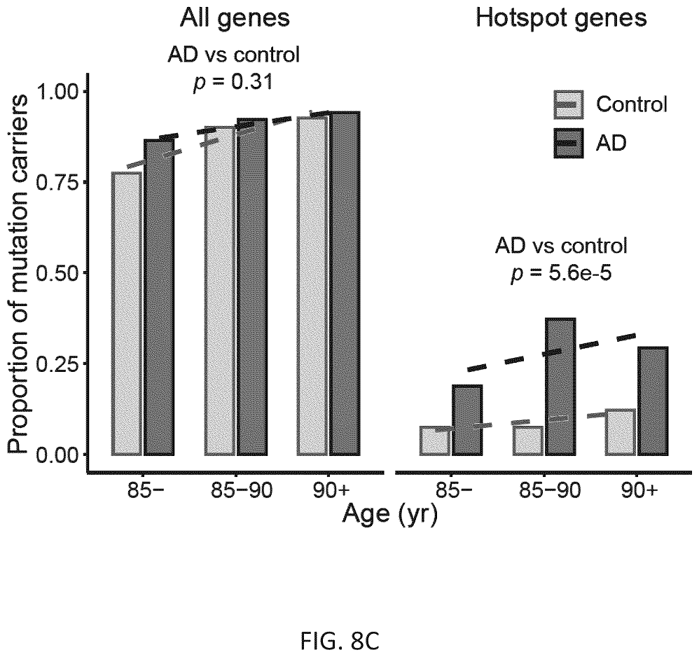

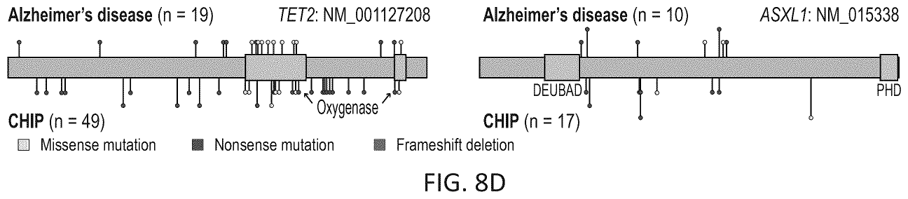

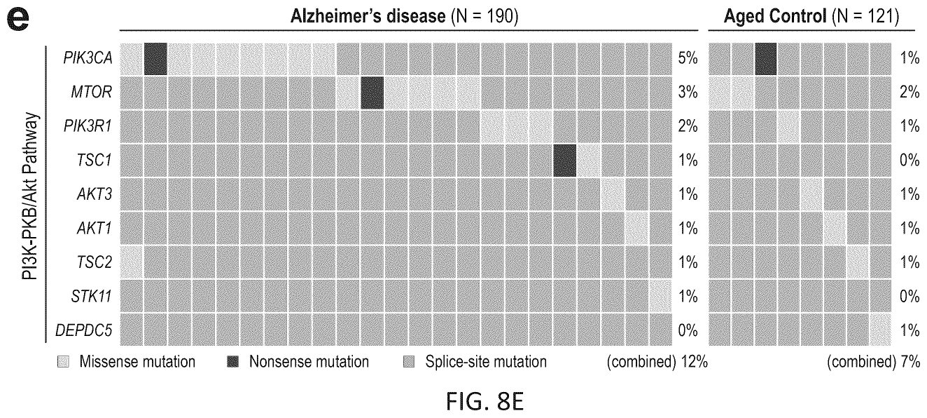

As an orthogonal and more sensitive approach to examining the mutational burden in proliferation-related genes in Alzheimer's disease, a gene panel was designed covering 149 genes frequently mutated in cancer and clonal hematopoiesis of indeterminate potential events with unique molecular identifier barcoding (Table 4). The gene panel was applied to sequence the prefrontal cortex of 190 Alzheimer's disease patients and 121 matched controls from the ROSMAP cohort at an average sequencing depth of >1000× after unique molecular identifier collapsing (Table 5; FIGS. 7A-7B). By exponentially reducing base-calling errors when generating the consensus sequence from multiple reads derived from the same original DNA molecule, this unique molecular identifier-based panel sequencing allowed detection of somatic mutations with mutant allele fractions as low as 0.1% (FIGS. 7C-7D)). This panel sequencing had much higher sensitivity and precision than previous methods without consensus error correction. Using this customized computational pipeline, 199 sSNVs and 13 somatic indels (sIndels) were identified that were exclusively present in a single DNA sample (the “stringent” list). To increase the detection power, recurrent mutations were allowed when they were specifically enriched in Alzheimer's disease or in control samples, which expanded the list to 1001 sSNVs and 20 sIndels, respectively (the “sensitive” list; FIGS. 8A-8B). 22 sSNVs were randomly selected with a range of mutant allele fractions for validation using amplicon sequencing, along with 17 potentially pathogenic sSNVs identified in Alzheimer's disease brains that were predicted to be deleterious, and all of the 10 frameshift sIndels in the “sensitive” list. Thirty-five of 39 (90) tested sSNVs and 8 of 10 (80%) sIndels were successfully validated in newly extracted DNA samples from the corresponding prefrontal cortex samples, confirming the high accuracy of the somatic mutation calling strategy even for those with mutant allele fractions as low as 0.1% (FIGS. 7E-7G).

TABLE 4

| Symbol | Gene Name | Ensembl ID | Oncogene | TSG |
|---|---|---|---|---|
| ABL1 | ABL proto-oncogene 1, non-receptor tyrosine kinase | ENSG00000097007 | Yes | |
| AKT1 | AKT serine/threonine kinase 1 | ENSG00000142208 | Yes | |
| AKT3 | AKT serine/threonine kinase 3 | ENSG00000117020 | Yes | |
| ALK | ALK receptor tyrosine kinase | ENSG00000171094 | Yes | |
| APC | APC, WNT signaling pathway regulator | ENSG00000134982 | | Yes |
| AR | androgen receptor | ENSG00000169083 | Yes | |
| ASXL1 | ASXL transcriptional regulator 1 | ENSG00000171456 | | Yes |
| ATM | ATM serine/threonine kinase | ENSG00000149311 | | Yes |
| ATRX | ATRX, chromatin remodeler | ENSG00000085224 | | Yes |
| BCR | BCR, RhoGEF and GTPase activating protein | ENSG00000186716 | | |
| BRAF | B-Raf proto-oncogene, serine/threonine kinase | ENSG00000157764 | Yes | |
| CALM3 | calmodulin 3 | ENSG00000160014 | | |
| CBL | Cbl proto-oncogene | ENSG00000110395 | | |
| CCND2 | cyclin D2 | ENSG00000118971 | Yes | |
| CD28 | CD28 molecule | ENSG00000178562 | Yes | |
| CDC73 | cell division cycle 73 | ENSG00000134371 | | Yes |
| CDH1 | cadherin 1 | ENSG00000039068 | | Yes |
| CDK4 | cyclin dependent kinase 4 | ENSG00000135446 | Yes | |
| CDK6 | cyclin dependent kinase 6 | ENSG00000105810 | Yes | |
| CDKN2A | cyclin dependent kinase inhibitor 2A | ENSG00000147889 | | Yes |
| CDKN2B | cyclin dependent kinase inhibitor 2B | ENSG00000147883 | | |
| CDKN2C | cyclin dependent kinase inhibitor 2C | ENSG00000123080 | | Yes |
| CHEK2 | checkpoint kinase 2 | ENSG00000183765 | | Yes |
| CIC | capicua transcriptional repressor | ENSG00000079432 | | |
| CSF1R | colony stimulating factor 1 receptor | ENSG00000182578 | Yes | |
| CTNNB1 | catenin beta 1 | ENSG00000168036 | Yes | |
| DDX3X | DEAD-box helicase 3 X-linked | ENSG00000215301 | | Yes |
| DEPDC5 | DEP domain containing 5 | ENSG00000100150 | | |
| DICER1 | dicer 1, ribonuclease III | ENSG00000100697 | | Yes |
| DNMT3A | DNA methyltransferase 3 alpha | ENSG00000119772 | | Yes |
| EGFR | epidermal growth factor receptor | ENSG00000146648 | Yes | |
| EPCAM | epithelial cell adhesion molecule | ENSG00000119888 | | |
| EPPK1 | epiplakin 1 | ENSG00000261150 | | |
| ERBB2 | erb-b2 receptor tyrosine kinase 2 | ENSG00000141736 | Yes | |
| ERBB4 | erb-b2 receptor tyrosine kinase 4 | ENSG00000178568 | | |
| EZH2 | enhancer of zeste 2 polycomb repressive complex 2 | ENSG00000106462 | | |
| | subunit | | | |
| FBXW7 | F-box and WD repeat domain containing 7 | ENSG00000109670 | | Yes |
| FGFR1 | fibroblast growth factor receptor 1 | ENSG00000077782 | Yes | |
| FGFR2 | fibroblast growth factor receptor 2 | ENSG00000066468 | Yes | |
| FGFR3 | fibroblast growth factor receptor 3 | ENSG00000068078 | Yes | |
| FLT3 | fms related tyrosine kinase 3 | ENSG00000122025 | Yes | |
| FUBP1 | far upstream element binding protein 1 | ENSG00000162613 | Yes | |
| FYN | FYN proto-oncogene, Src family tyrosine kinase | ENSG00000010810 | | |
| GLI3 | GLI family zinc finger 3 | ENSG00000106571 | | |
| GNA11 | G protein subunit alpha 11 | ENSG00000088256 | Yes | |
| GNAQ | G protein subunit alpha q | ENSG00000156052 | Yes | |
| GNAS | GNAS complex locus | ENSG00000087460 | Yes | |
| GNB1 | G protein subunit beta 1 | ENSG00000078369 | | |
| GPC3 | glypican 3 | ENSG00000147257 | | |
| H3F3A | H3 histone family member 3A | ENSG00000163041 | Yes | |
| HIF1A | hypoxia inducible factor 1 subunit alpha | ENSG00000100644 | Yes | |
| HIST1H3B | histone cluster 1 H3 family member b | ENSG00000274267 | Yes | |
| HNF1A | HNF1 homeobox A | ENSG00000135100 | | Yes |
| HRAS | HRas proto-oncogene, GTPase | ENSG00000174775 | Yes | |
| IDH1 | isocitrate dehydrogenase (NADP(+)) 1, cytosolic | ENSG00000138413 | Yes | |
| IDH2 | isocitrate dehydrogenase (NADP(+)) 2, mitochondrial | ENSG00000182054 | Yes | |
| JAK2 | Janus kinase 2 | ENSG00000096968 | Yes | |
| JAK3 | Janus kinase 3 | ENSG00000105639 | Yes | |
| KDR | kinase insert domain receptor | ENSG00000128052 | Yes | |
| KIF7 | kinesin family member 7 | ENSG00000166813 | | |
| KIT | KIT proto-oncogene receptor tyrosine kinase | ENSG00000157404 | Yes | |
| KLF4 | Kruppel like factor 4 | ENSG00000136826 | | |
| KMT2C | lysine methyltransferase 2C | ENSG00000055609 | | Yes |
| KMT2D | lysine methyltransferase 2D | ENSG00000167548 | | |
| KRAS | KRAS proto-oncogene, GTPase | ENSG00000133703 | Yes | |
| MAP2K1 | mitogen-activated protein kinase kinase 1 | ENSG00000169032 | Yes | |
| MAP2K2 | mitogen-activated protein kinase kinase 2 | ENSG00000126934 | Yes | |
| MAP3K1 | mitogen-activated protein kinase kinase kinase 1 | ENSG00000095015 | | |
| MAP4K1 | mitogen-activated protein kinase kinase kinase | ENSG00000104814 | | |
| | kinase 1 | | | |
| MDM1 | Mdm1 nuclear protein | ENSG00000111554 | | |
| MDM2 | MDM2 proto-oncogene | ENSG00000135679 | Yes | |
| MED12 | mediator complex subunit 12 | ENSG00000184634 | | Yes |
| MEN1 | menin 1 | ENSG00000133895 | | Yes |
| MET | MET proto-oncogene, receptor tyrosine kinase | ENSG00000105976 | Yes | |
| MIOS | meiosis regulator for oocyte development | ENSG00000164654 | | |
| MLH1 | mutL homolog 1 | ENSG00000076242 | | Yes |
| MPL | MPL proto-oncogene, thrombopoietin receptor | ENSG00000117400 | Yes | |
| MSH2 | mutS homolog 2 | ENSG00000095002 | | Yes |
| MSH6 | mutS homolog 6 | ENSG00000116062 | | Yes |
| MTOR | mechanistic target of rapamycin kinase | ENSG00000198793 | Yes | |
| MYC | MYC proto-oncogene, bHLH transcription factor | ENSG00000136997 | Yes | |
| MYCN | MYCN proto-oncogene, bHLH transcription factor | ENSG00000134323 | Yes | |
| NF1 | neurofibromin 1 | ENSG00000196712 | | Yes |
| NF2 | neurofibromin 2 | ENSG00000186575 | | Yes |
| NOP53 | NOP53 ribosome biogenesis factor | ENSG00000105373 | | |
| NOTCH3 | notch 3 | ENSG00000074181 | | |
| NPM1 | nucleophosmin 1 | ENSG00000181163 | Yes | |
| NPRL2 | NPR2 like, GATOR1 complex subunit | ENSG00000114388 | | |
| NPRL3 | NPR3 like, GATOR1 complex subunit | ENSG00000103148 | | |
| NRAS | NRAS proto-oncogene, GTPase | ENSG00000213281 | Yes | |
| NRGN | neurogranin | ENSG00000154146 | | |
| NSD1 | nuclear receptor binding SET domain protein 1 | ENSG00000165671 | | |
| OFD1 | OFD1, centriole and centriolar satellite protein | ENSG00000046651 | | |
| PBRM1 | polybromo 1 | ENSG00000163939 | | Yes |
| PDGFRA | platelet derived growth factor receptor alpha | ENSG00000134853 | Yes | |
| PHOX2B | paired like homeobox 2B | ENSG00000109132 | | Yes |
| PIK3CA | phosphatidylinositol-4,5-bisphosphate 3-kinase | ENSG00000121879 | Yes | |
| | catalytic subunit alpha | | | |
| PIK3CG | phosphatidylinositol-4,5-bisphosphate 3-kinase | ENSG00000105851 | | |
| | catalytic subunit gamma | | | |
| PIK3R1 | phosphoinositide-3-kinase regulatory subunit 1 | ENSG00000145675 | | Yes |
| PIK3R2 | phosphoinositide-3-kinase regulatory subunit 2 | ENSG00000105647 | | |
| PLEKHS1 | pleckstrin homology domain containing S1 | ENSG00000148735 | | |
| PMS2 | PMS1 homolog 2, mismatch repair system | ENSG00000122512 | | Yes |
| | component | | | |
| PPM1D | protein phosphatase, Mg2+/Mn2+ dependent 1D | ENSG00000170836 | Yes | |
| PRKAR1A | protein kinase cAMP-dependent type I regulatory | ENSG00000108946 | | |
| | subunit alpha | | | |
| PRNP | prion protein | ENSG00000171867 | | |
| PTCH1 | patched 1 | ENSG00000185920 | | Yes |
| PTEN | phosphatase and tensin homolog | ENSG00000171862 | | Yes |
| PTPN11 | protein tyrosine phosphatase, non-receptor type 11 | ENSG00000179295 | Yes | |
| RAB39A | RAB39A, member RAS oncogene family | ENSG00000179331 | | |
| RAF1 | Raf-1 proto-oncogene, serine/threonine kinase | ENSG00000132155 | Yes | |
| RB1 | RB transcriptional corepressor 1 | ENSG00000139687 | | Yes |
| RET | ret proto-oncogene | ENSG00000165731 | Yes | |
| RHOA | ras homolog family member A | ENSG00000067560 | | |
| RIT1 | Ras like without CAAX 1 | ENSG00000143622 | | |
| RPL5 | ribosomal protein L5 | ENSG00000122406 | | Yes |
| RPSAP58 | ribosomal protein SA pseudogene 58 | ENSG00000288920 | | |
| SDHD | succinate dehydrogenase complex subunit D | ENSG00000204370 | | Yes |
| SEC13 | SEC13 homolog, nuclear pore and COPII coat | ENSG00000157020 | | |
| | complex component | | | |
| SEH1L | SEH1 like nucleoporin | ENSG00000085415 | | |
| SETD2 | SET domain containing 2 | ENSG00000181555 | | Yes |
| SF3B1 | splicing factor 3b subunit 1 | ENSG00000115524 | Yes | |
| SHOC2 | SHOC2, leucine rich repeat scaffold protein | ENSG00000108061 | | |
| SMAD3 | SMAD family member 3 | ENSG00000166949 | | Yes |
| SMAD4 | SMAD family member 4 | ENSG00000141646 | | Yes |
| SMARCB1 | SWI/SNF related, matrix associated, actin dependent | ENSG00000099956 | | Yes |
| | regulator of chromatin, subfamily b, member 1 | | | |
| SMO | smoothened, frizzled class receptor | ENSG00000128602 | Yes | |
| SOS1 | SOS Ras/Rac guanine nucleotide exchange factor 1 | ENSG00000115904 | | |
| SPRED1 | sprouty related EVH1 domain containing 1 | ENSG00000166068 | | |
| SRC | SRC proto-oncogene, non-receptor tyrosine kinase | ENSG00000197122 | Yes | |
| SRSF2 | serine and arginine rich splicing factor 2 | ENSG00000161547 | Yes | |
| STAG2 | stromal antigen 2 | ENSG00000101972 | | Yes |
| STAT3 | signal transducer and activator of transcription 3 | ENSG00000168610 | Yes | |
| STAT5B | signal transducer and activator of transcription 5B | ENSG00000173757 | | |
| STK11 | serine/threonine kinase 11 | ENSG00000118046 | | Yes |
| SUFU | SUFU negative regulator of hedgehog signaling | ENSG00000107882 | | Yes |
| TERT | telomerase reverse transcriptase | ENSG00000164362 | | |
| TET2 | tet methylcytosine dioxygenase 2 | ENSG00000168769 | | Yes |
| TNFAIP3 | TNF alpha induced protein 3 | ENSG00000118503 | | Yes |
| TNNI3 | troponin I3, cardiac type | ENSG00000129991 | | |
| TP53 | tumor protein p53 | ENSG00000141510 | | |
| TSC1 | TSC complex subunit 1 | ENSG00000165699 | | Yes |
| TSC2 | TSC complex subunit 2 | ENSG00000103197 | | Yes |
| TSHZ2 | teashirt zinc finger homeobox 2 | ENSG00000182463 | | |
| U2AF1 | U2 small nuclear RNA auxiliary factor 1 | ENSG00000160201 | Yes | |
| VHL | von Hippel-Lindau tumor suppressor | ENSG00000134086 | | Yes |
| WDR24 | WD repeat domain 24 | ENSG00000127580 | | |
| WDR59 | WD repeat domain 59 | ENSG00000103091 | | |
| WDR74 | WD repeat domain 74 | ENSG00000133316 | | |
| WT1 | Wilms tumor 1 | ENSG00000184937 | | |

TABLE 5

| | | | | | | | Sequencing | Sequencing |
|---|---|---|---|---|---|---|---|---|
| | | | | | | | depth after | coverage |
| | Brain | | # of | Female | Age | PMI | UMI | with >500X |
| Dataset | region | Diagnosis | samples | % | (yr) | (hr) | aggregation | depth (Mb) |
| ROSMAP | Prefrontal | Healthy | 123 | 66.67% | 85.93 | 8.63 | 1264 ± 275 | 0.58 ± 0.06 |
| | cortex | control | | | ± | ± | | |
| | | | | | 4.87 | 5.27 | | |
| | | Alzheimer's | 190 | 68.95% | 87.74 | 7.84 | 1305 ± 277 | 0.58 ± 0.06 |
| | | disease | | | ± | ± | | |
| | | | | | 3.52 | 5.05 | | |

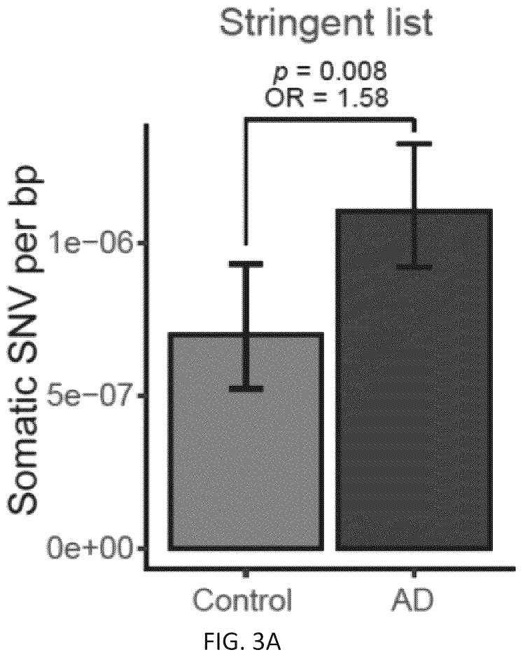

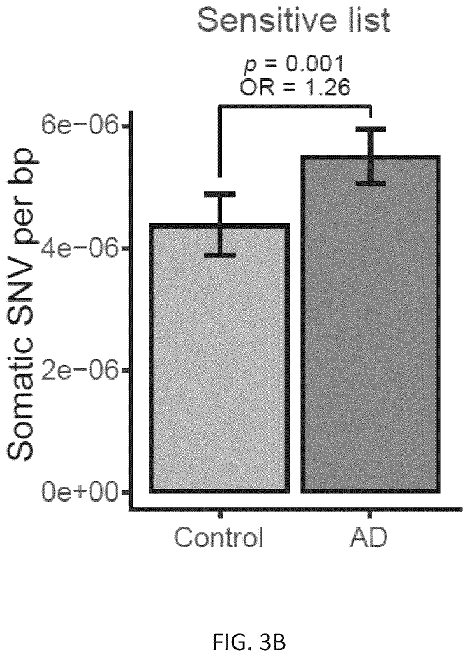

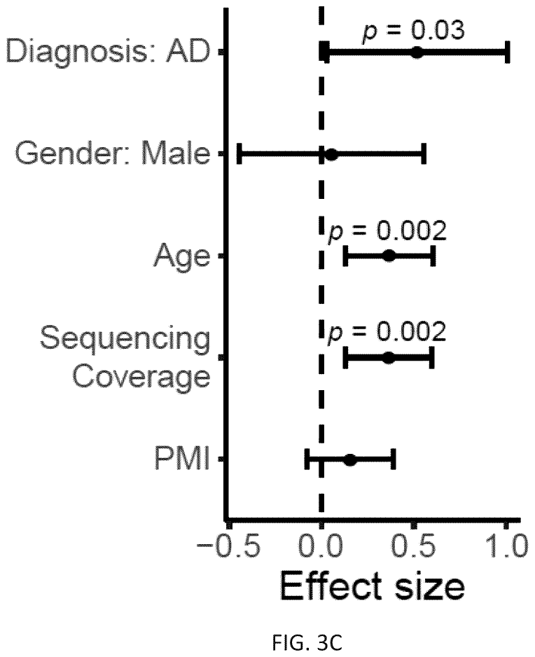

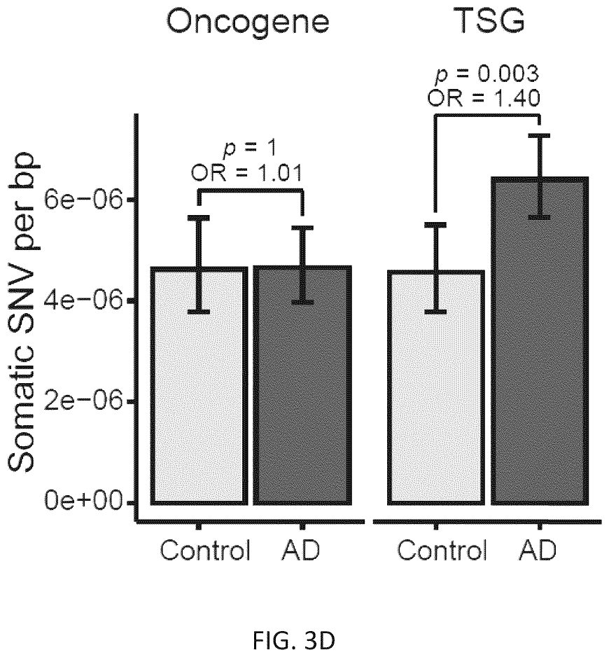

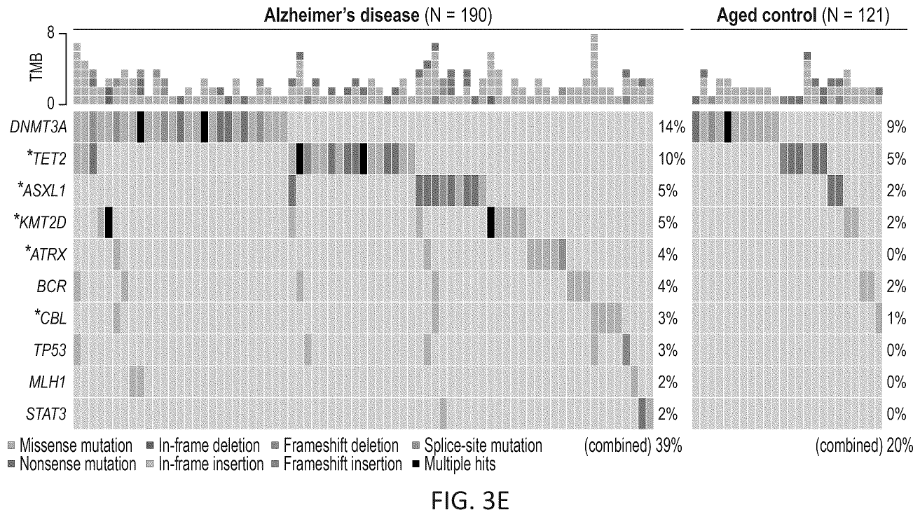

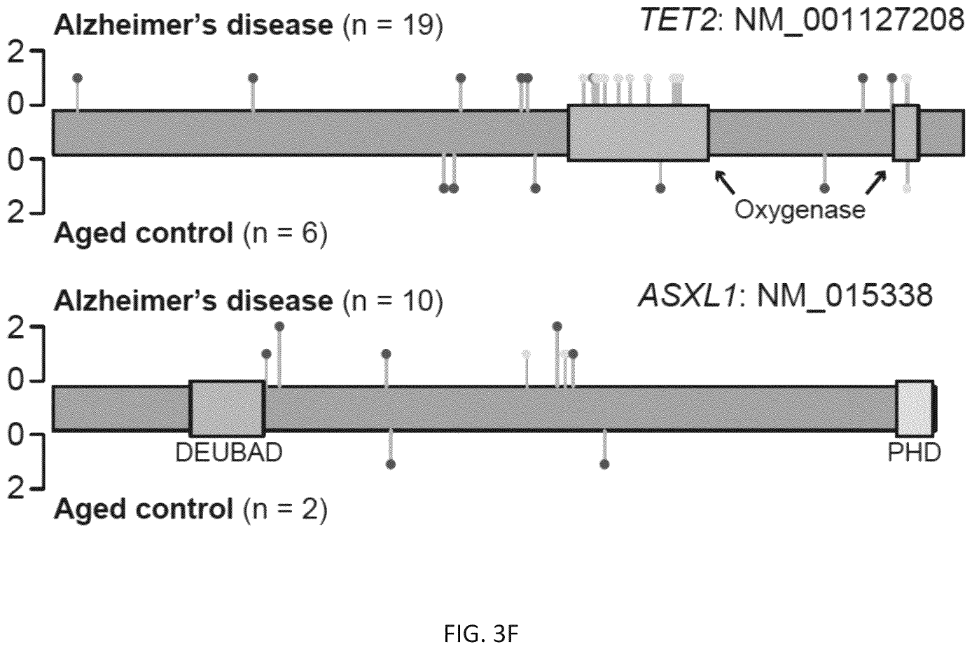

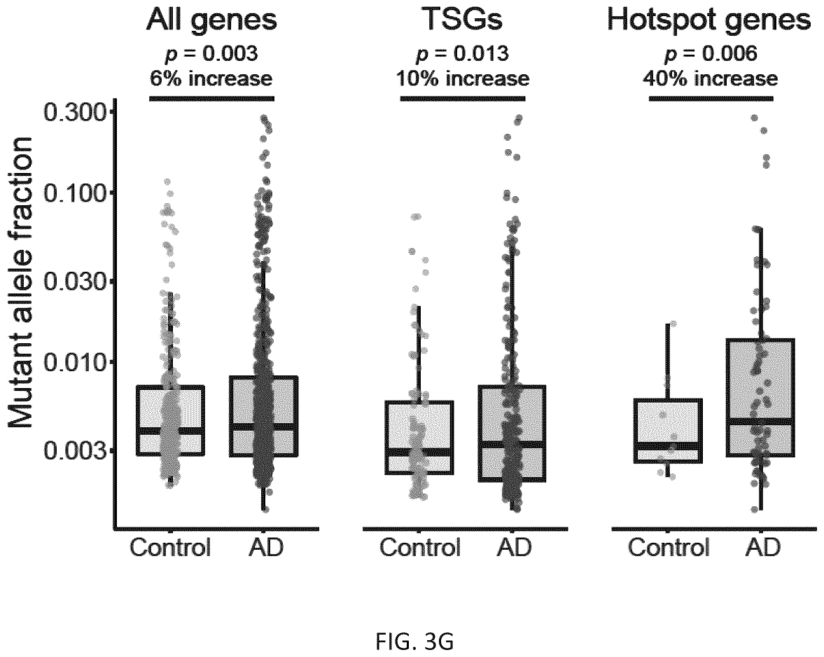

With similar sequencing depth and coverage between Alzheimer's disease and control prefrontal cortex samples (FIGS. 7A-7B), the stringent pipeline revealed that Alzheimer's disease brains harbored significantly more sSNVs among the 149 proliferation-related genes than aged-matched controls (FIG. 3A; p=0.008; OR=1.6). When using the sensitive pipeline which allows recurrent mutations, the sSNV increase in Alzheimer's disease brains became even more significant (FIG. 3B; p=0.001; OR=1.3), and this pattern remained significant after controlling for confounding factors including age, gender, sequencing coverage, and post-mortem interval (FIG. 3C; p=0.03).

In addition to the Alzheimer's disease effect, that Alzheimer's disease brains harbored significantly more sSNVs among the 149 proliferation-related genes than aged-matched controls (FIG. 3C; p=0.002) and the proportion of sSNV carriers (FIG. 8C), indicating a likely age-associated accumulation of somatic mutations in proliferation-related genes in both normal and diseased brains. Interestingly, when proliferation-related genes were further divided into (proto-)oncogenes and tumor suppressor genes (TSGs), the greater sSNV burden in Alzheimer's disease was observed for tumor suppressor genes but not for oncogenes (FIG. 3D). Considering that tumor suppressor genes lead to proliferation when they are inactivated by loss-of-function mutations throughout the gene body, but oncogenes are usually only activated by specific gain-of-function alleles affecting critical domains, the results indicated that the majority of sSNVs are associated with Alzheimer's disease by a loss-of-function manner in tumor suppressor genes. Besides sSNVs, more frameshift sIndels were observed in Alzheimer's disease brains (7 in Alzheimer's disease versus 2 in control; Table 5).

Examination of the somatic mutation burden at the individual-gene level revealed that sSNVs in the top 10 most commonly mutated genes were found in 39% of the Alzheimer's disease patients compared to only 20% of the aged controls (FIG. 3E). Five “hotspot” genes—TET2, ASXL1, KMT2D, ATRX, and CBL harbored nominally more somatic mutations in Alzheimer's disease brains than controls (FIG. 3E; p<0.05), though these individual gene burdens were not significant after multiple hypothesis testing for 149 genes. All “hotspot” genes represented critical tumor suppressor genes. Most Alzheimer's disease somatic mutations in ASXL1 were nonsense mutations broadly distributed across the encoded protein, including two recurrent alleles observed in multiple Alzheimer's disease patients, similar to what was seen in clonal hematopoiesis of indeterminate potential events of blood. Alzheimer's disease patients showed missense mutations in TET2 that clustered in its critical oxygenase domains (FIG. 3F), a similar mutational pattern to that seen in clonal hematopoiesis of indeterminate potential (FIG. 8D) but not seen in aged controls. Somatic mutations in Alzheimer's disease brains showed significantly higher mutant allele fractions than did mutations in control brains, especially in the five hotspot genes where the average mutant allele fraction was 40% increased, indicating that many somatic mutations found in Alzheimer's disease drive the clonal expansion of cells that carry them (FIG. 3G) to a greater extent than in control brains. In addition to individual genes, Alzheimer's disease patients had significantly more somatic mutations in PI3K-PKB/Akt pathway genes than did controls (FIG. 8E; p<0.05), a pathway that may be enriched with somatic mutations in Alzheimer's disease brains. Without intending to be bound by theory, the panel sequencing results revealed more frequent somatic mutations in proliferation-related genes of Alzheimer's disease brains, highlighting their potential roles in driving the clonal expansion of certain proliferating cell types during Alzheimer's disease pathogenesis.

### Example 4: Microglial Enrichment of Proliferation-Related Somatic Mutations

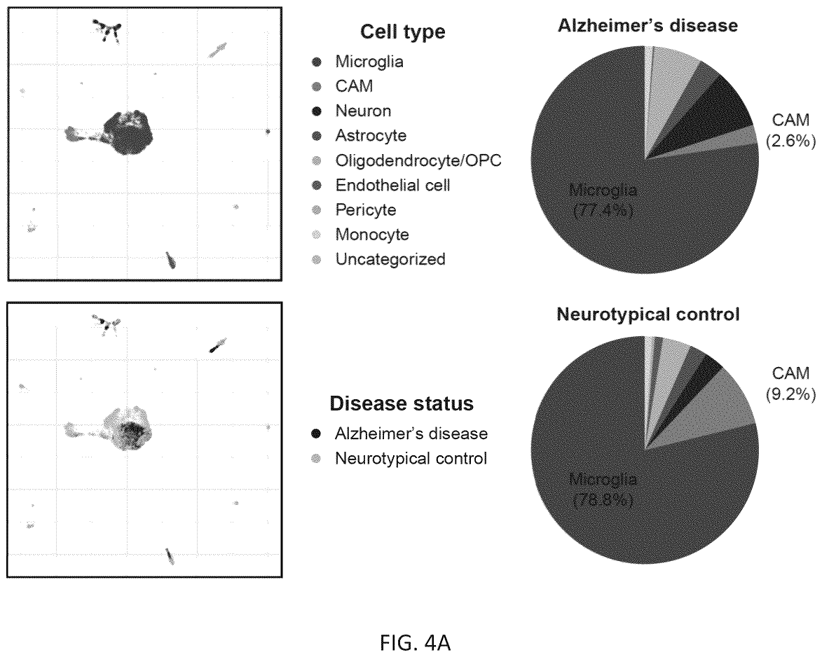

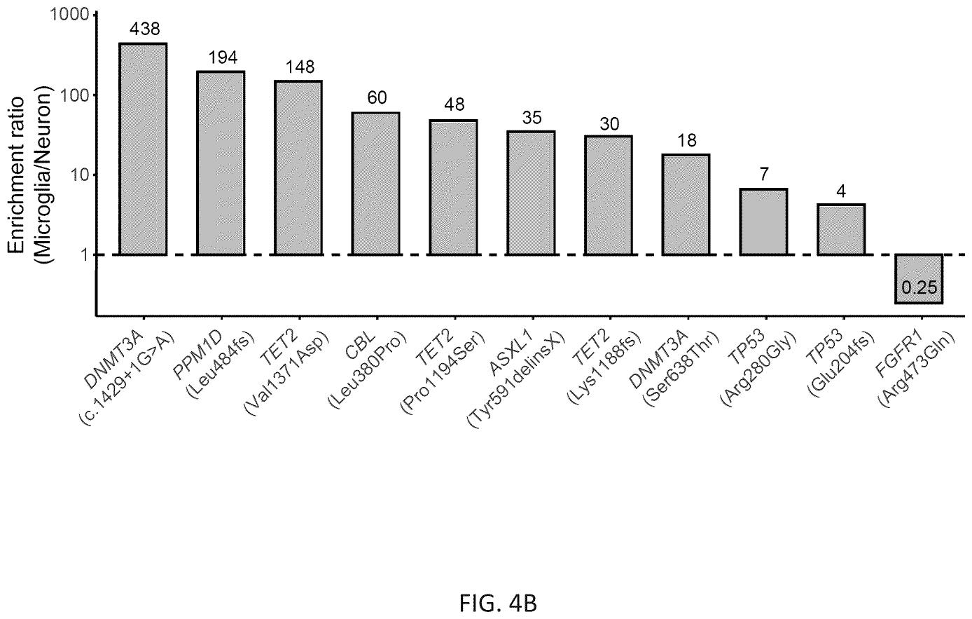

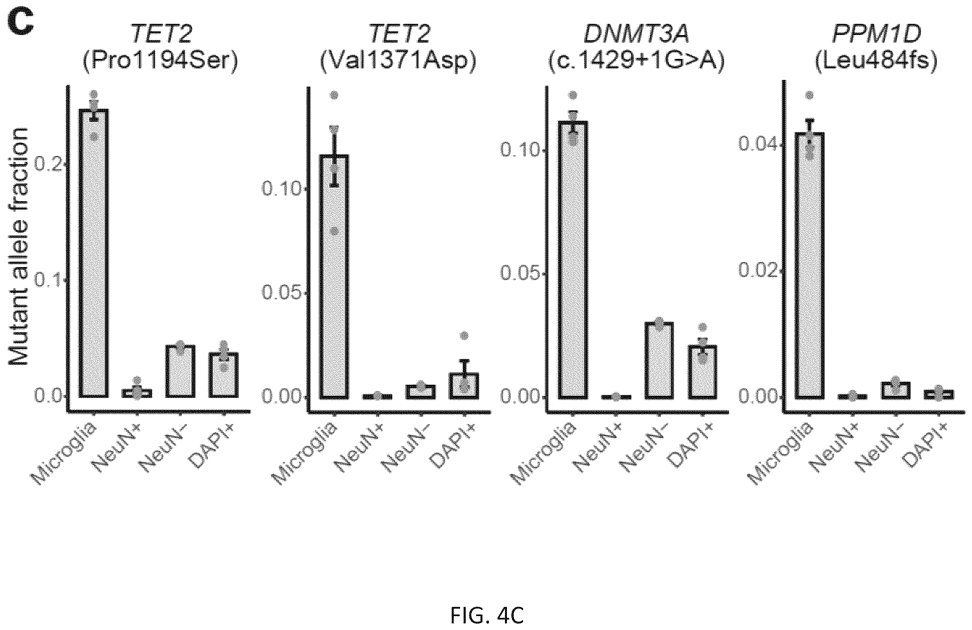

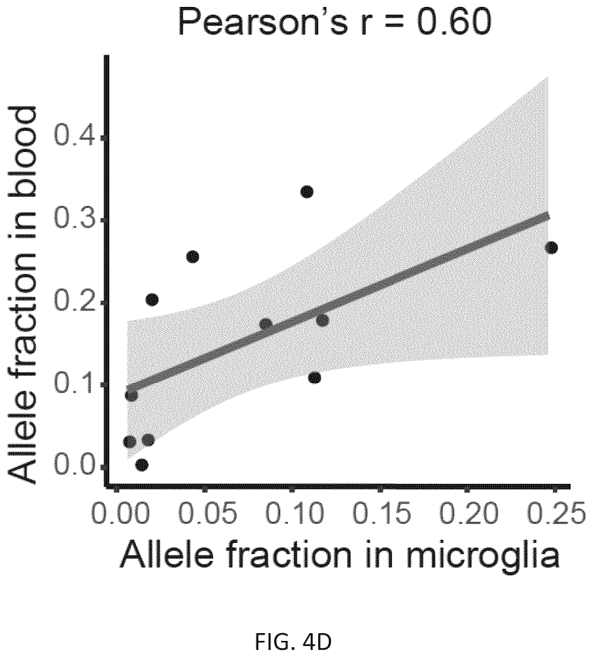

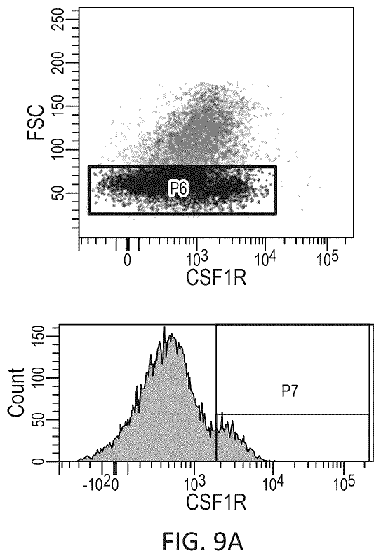

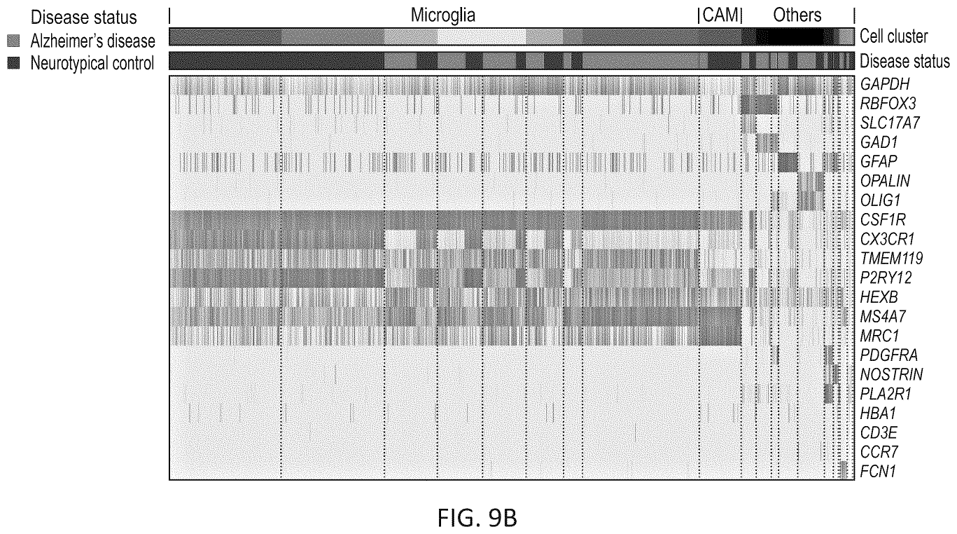

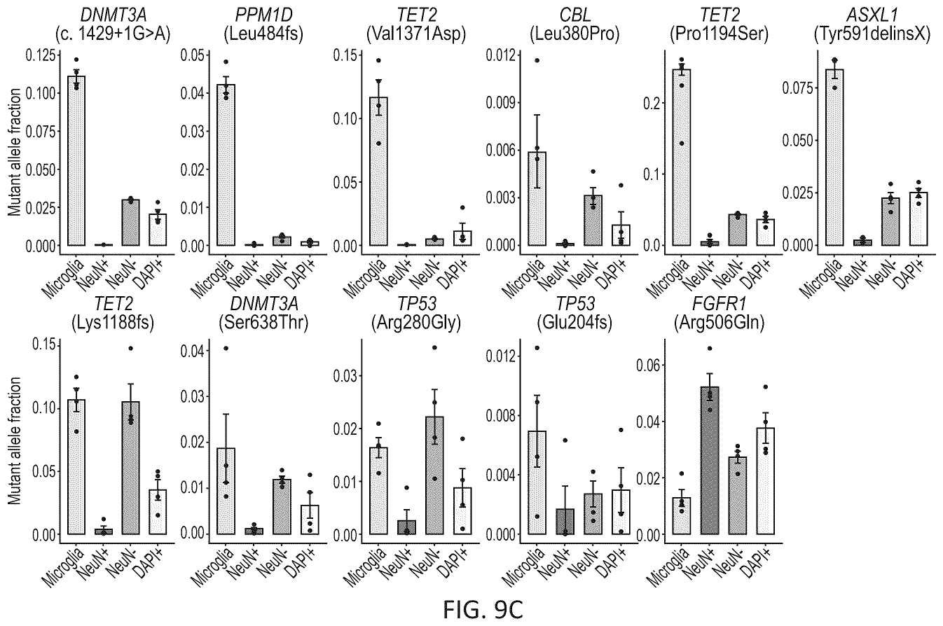

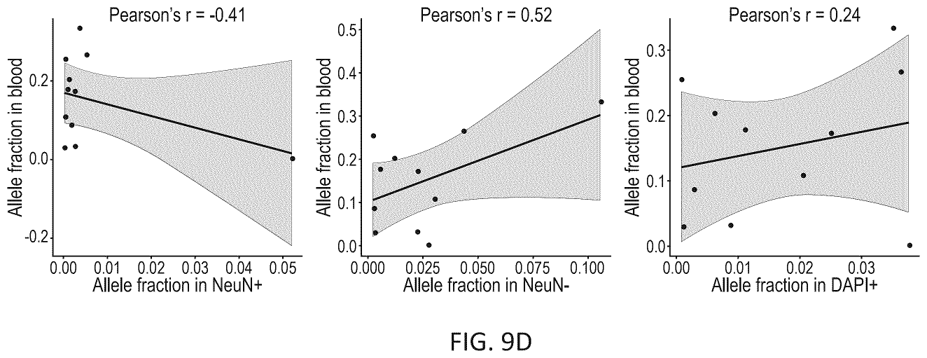

As clonal hematopoiesis of indeterminate potential mutations of the blood are commonly found in myeloid cells, microglia, which share a very early lineage with other myeloid cells, were hypothesized to be the carrier cells of these pathogenic mutations in Alzheimer's disease brains. To test this, a fluorescence-activated nuclei sorting method was developed to specifically isolate microglial nuclei from frozen postmortem brain tissues using an antibody targeting CSF1R (FIG. 9A), a cell surface marker for microglia. The subsequent single-nucleus RNA-seq (snRNAseq) result from Alzheimer's disease and control brains confirmed that about 80% of the sorted nuclei belonged to the microglial cluster (FIG. 4A), categorized by expression of microglia marker genes including CX3CR1, TMEM119, and P2RY12 (FIG. 9B). Interestingly, another 3-9% of the nuclei were classified as CNS-associated macrophages (CAMs) (FIG. 4A and FIG. 9B), a class of brain-resident myeloid cells with high expression of MS4A7 and MRC1, while the remaining cells represented scattered neural cells or pericytes. Both microglia and CNS-associated macrophages are brain-resident macrophages predominantly derived from the erythromyeloid progenitors during embryogenesis, but a contribution of hematopoietic-derived immune cells were found in the brain macrophage pool in adulthood.

Based on the predicted pathogenicity inferred from their mutation types, impacted genes, and population allele frequencies, 7 sSNVs and 4 sIndels identified from Alzheimer's disease brains were selected for cell type analysis, all of which were predicted to be deleterious for critical oncogenes or tumor suppressor genes. For each somatic mutation, amplicon sequencing was performed to measure the mutant allele fraction in four different populations of sorted cells: microglia (CSF1R+), neurons (NeuN+), glia and other nonneuronal cells (NeuN-), and all cells (DAPI+). In all but one somatic mutation, a 4- to 438-fold enrichment of mutant allele fractions was observed in microglia when compared to neurons sorted from the same brain region (FIG. 4B and FIG. 9C). In a splice-site sSNV in DNMT3A (c.1429+1G>A) and two deleterious missense sSNVs in TET2 (p.Pro1194Ser and p.Val1371Asp), >10% mutant allele fractions in microglia was observed, dramatically higher than the mutant allele fractions observed in neurons and other mixed cell populations (FIG. 4C; p<0.05), indicating that mutant cells constitute >20% of all microglia in the sample. Interestingly, the FGFR1 (p.Arg506Gln) variant, not known to stimulate cancer and predicted to result in lower levels of function, and the DNMT3A (c.1429+1G>A) variant co-exist in the same Alzheimer's disease prefrontal cortex sample, but the latter is almost exclusively present in microglia whereas the former is shared between microglia and neurons, indicating that the variants originated at different times (FIG. 4C).

Analysis of blood DNA from these 10 patients showed that 10/10 mutations that were enriched in microglia were also present in blood, with a weak positive correlation between mutant allele fractions in microglia and blood (p=0.052; FIG. 4D and FIG. 9D). Minimal blood contamination was confirmed in the unsorted bulk brains (as measured by RNA-seq analysis) and in the sorted microglial nuclei (FIG. 4A and FIG. 9B). Given the high mutant allele fractions of these mutations in microglia (typically >5%, equivalent to >10% cell fraction), the results indicate shared origins of these cancer-driver somatic mutations between the microglial and blood lineages.

### Example 5: Proliferation-Related Somatic Mutations in AD snRNAseq Data

To explore the effects of somatic mutations in microglia in Alzheimer's disease, a recent high-quality snRNAseq dataset of middle temporal gyrus neocortex samples obtained from Alzheimer's disease donors and age-matched controls was utilized, the Seattle Alzheimer's Disease Brain Cell Atlas (SEA-AD). Since somatic copy number variants (sCNVs) have also been associated with CHIP, generally disrupting specific genes also mutated by sSNV, it was hypothesized that Alzheimer's disease brains would also carry somatic copy number variants at loci associated with CHIP (CHIP-sCNV) in microglia-CNS-associated macrophages (CAMs).

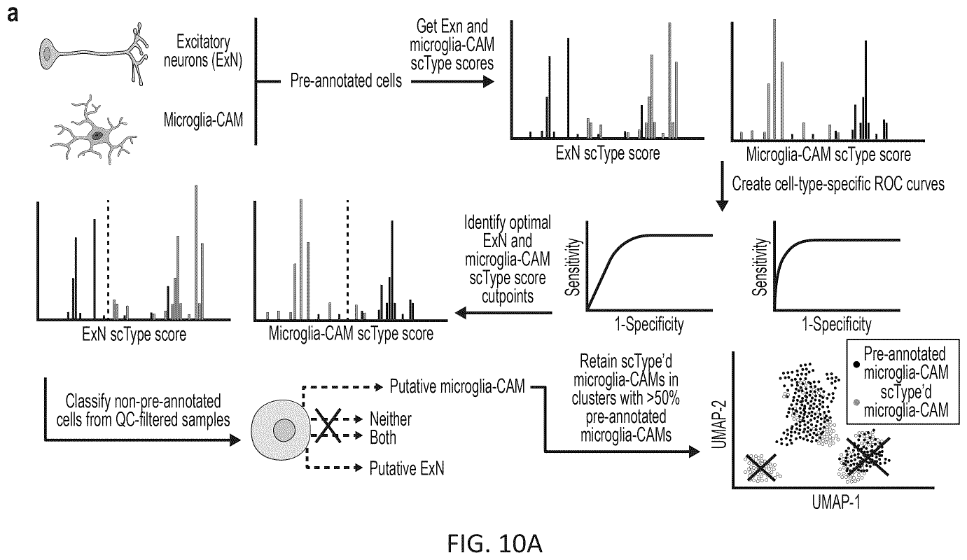

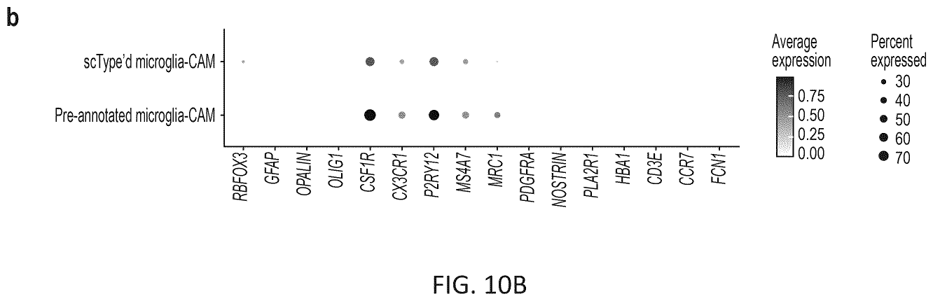

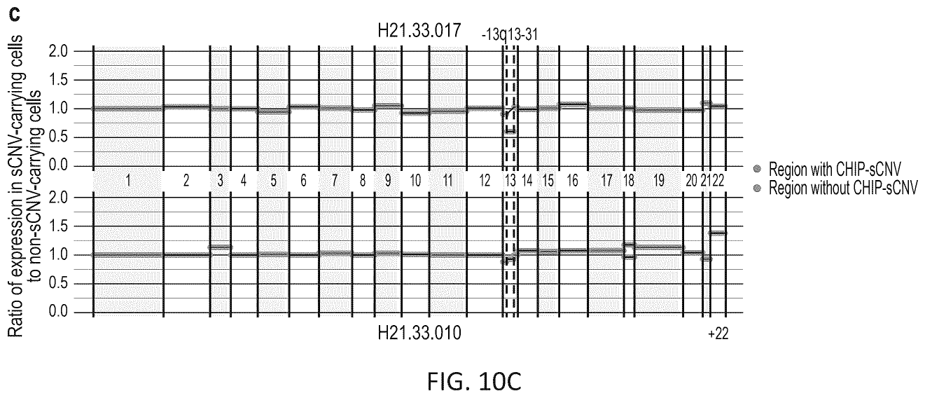

Cells were extracted that were annotated as microglia-perivascular macrophages (a subtype of CAMs, hereby called microglia-CAMs) or were identified as microglia-CAMs through automatic cell-typing with scType (FIGS. 10A-10B), and then called microglia-CAM-specific CHIP-sCNVs within SEA-AD using CONICSmat (Muller, S. et al. Bioinformatics 34, 3217-3219 (2018)) for all individuals with a consensus clinical diagnosis of Alzheimer's disease (n=31) or healthy, age-matched controls (n=31). CHIP-sCNVs were also called in excitatory neurons (ExNs), astrocytes, oligodendrocytes, or oligodendrocyte precursor cells (OPCs) and retained only CHIP-sCNVs that were not called in any of these other cell types from the same donor and which passed several stringent filtering criteria (FIG. 10C). A list of the regions/loci tested for CHIP-sCNVs is found in Table 6.

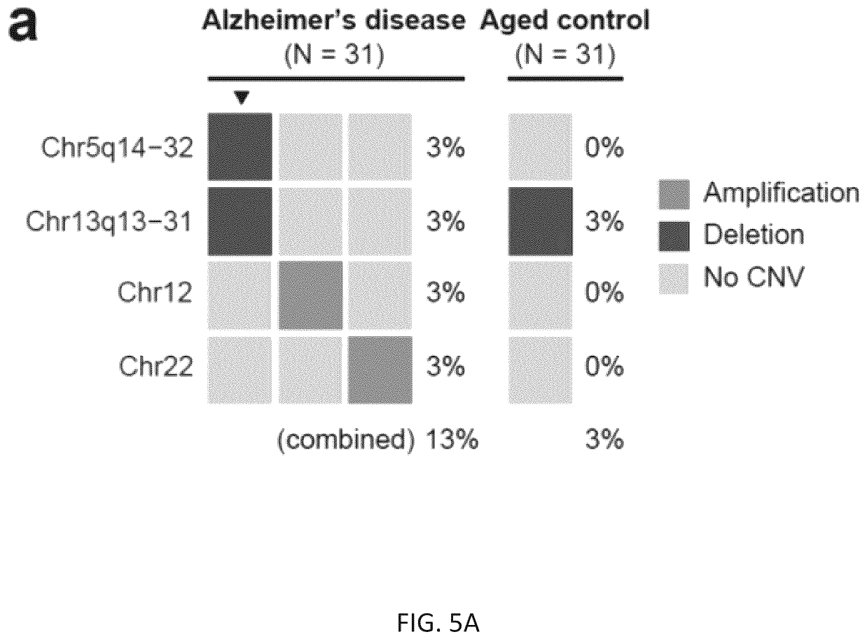

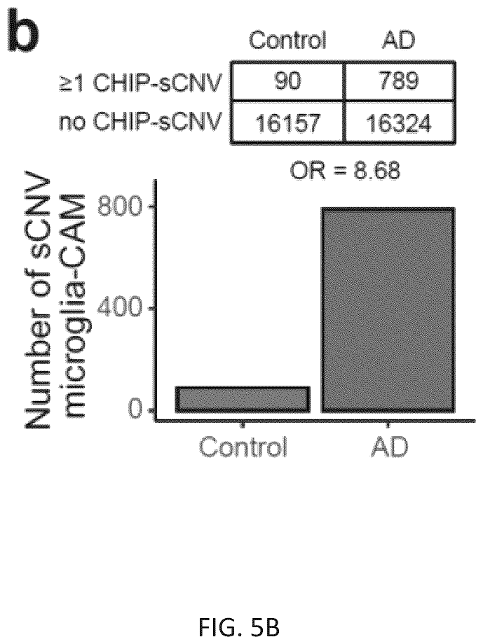

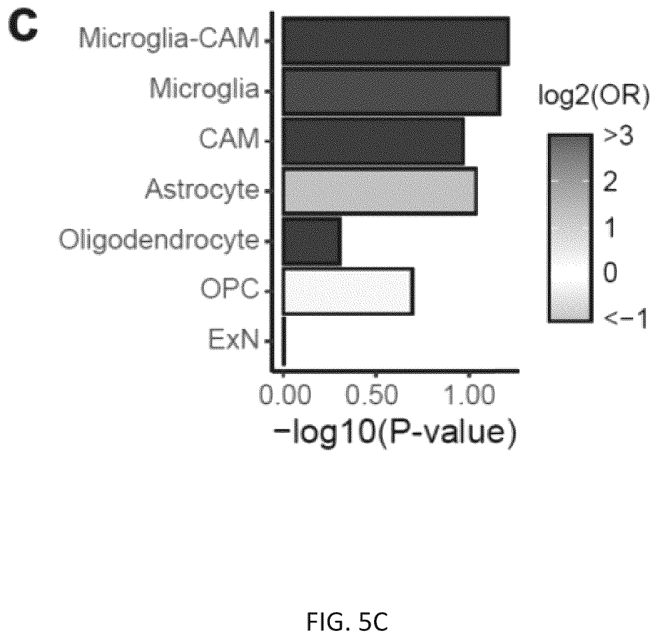

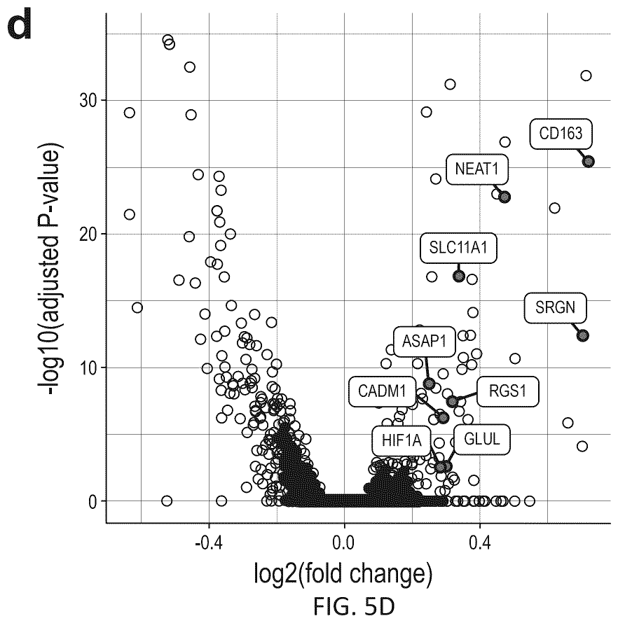

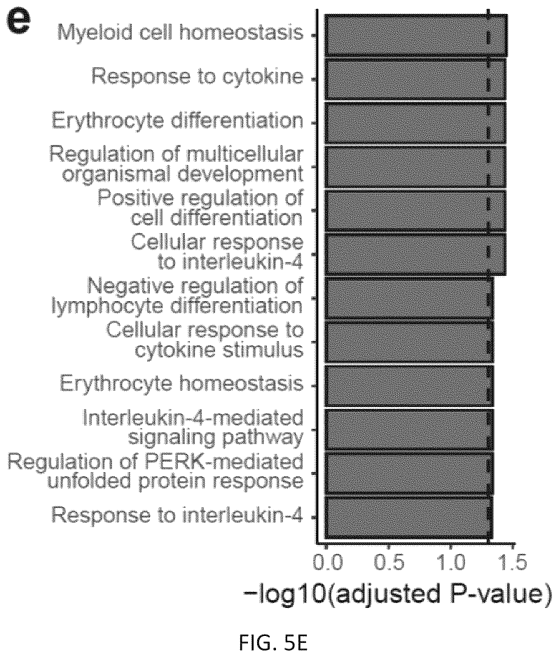

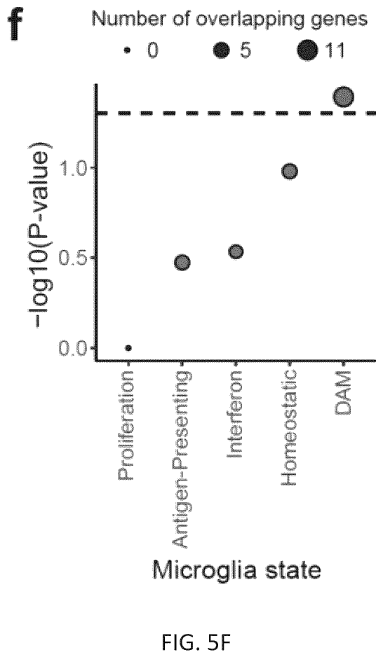

Alzheimer's disease brains harbored nominally more CHIP-sCNVs (4 in AD versus 1 in control; FIG. 5A) and nominally 8-fold more CHIP-sCNV-carrying microglia-CAMs (FIG. 5B; p=0.06, permutation test), though as expected, the SEA-AD sample size was too small for these differences to reach statistical significance. When microglia and CAM were analyzed separately, a stronger trend was observed in microglia than CAMs (FIG. 5C; p=0.07 and 0.11, permutation test). An increasing trend of CHIP-sCNV was also observed in AD individuals versus controls in astrocytes, but not in oligodendrocytes, OPCs, and ExNs (FIG. 5C).

TABLE 6

| Region ID | Chromosome | Start | End | Length | Type |
|---|---|---|---|---|---|
| 3q | 3 | 93655574 | 198295559 | 104639985 | Amplification |
| 14q | 14 | 18173523 | 107043718 | 88870195 | Amplification |
| 20q11-13 | 20 | 28100000 | 64444167 | 36344167 | Deletion |
| 14q11 | 14 | 17200000 | 24100000 | 6900000 | Deletion |
| 2p23 | 2 | 23800000 | 31800000 | 8000000 | Deletion |
| 9q31 | 9 | 99800000 | 112100000 | 12300000 | Deletion |
| 13q13-31 | 13 | 31600000 | 94400000 | 62800000 | Deletion |
| 5q14-32 | 5 | 77600000 | 150400000 | 72800000 | Deletion |
| 6q16-24 | 6 | 92500000 | 148500000 | 56000000 | Deletion |
| 11q14-23 | 11 | 77400000 | 121300000 | 43900000 | Deletion |
| 7q32-36 | 7 | 127500000 | 159345973 | 31845973 | Deletion |
| 3p13 | 3 | 69700000 | 74100000 | 4400000 | Deletion |
| 22q12 | 22 | 25500000 | 37200000 | 11700000 | Deletion |
| 17p13-11 | 17 | 0 | 25100000 | 25100000 | Deletion |
| 21q22 | 21 | 30200000 | 46709983 | 16509983 | Deletion |
| 10q25-26 | 10 | 104000000 | 117300000 | 13300000 | Deletion |
| 15 | 15 | 0 | 101991189 | 101991189 | Amplification |
| 21 | 21 | 0 | 46709983 | 46709983 | Amplification |
| 22 | 22 | 0 | 50818468 | 50818468 | Amplification |
| 18 | 18 | 0 | 80373285 | 80373285 | Amplification |
| 12 | 12 | 0 | 133275309 | 133275309 | Amplification |
| 8 | 8 | 0 | 145138636 | 145138636 | Amplification |

### Example 6: Transcriptional Effect of Somatic Mutations in AD Microglia

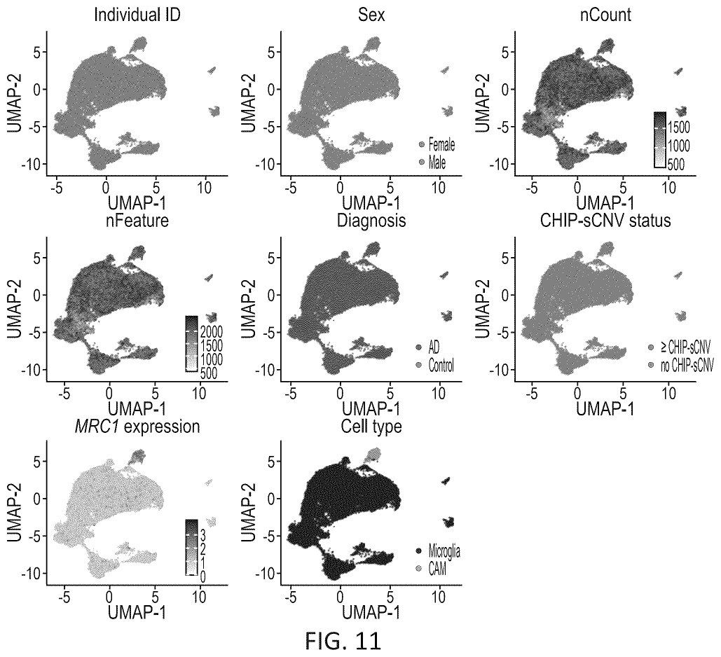

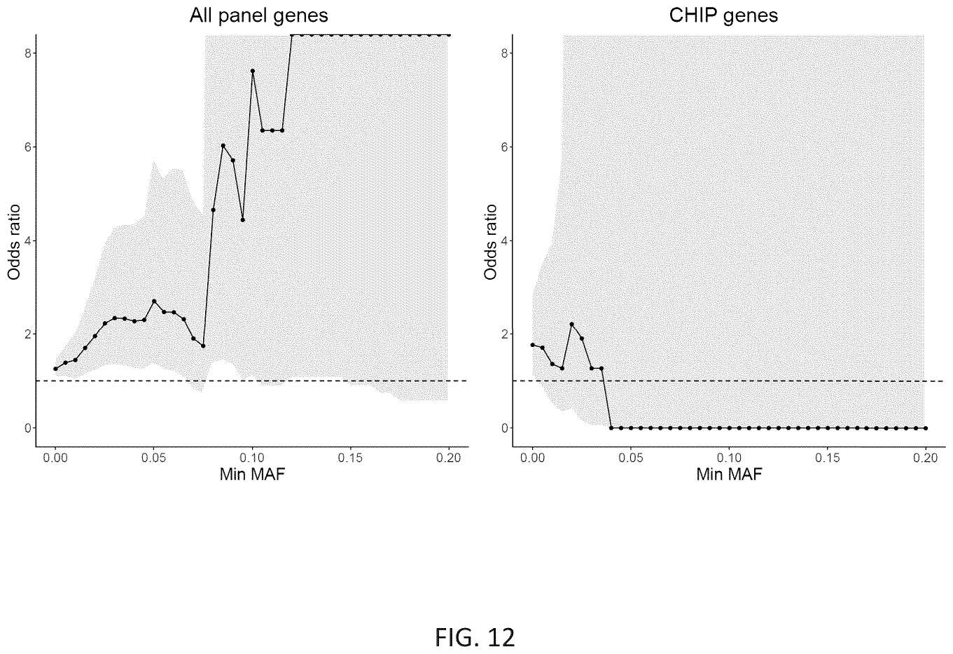

The SEA-AD sample size is consistent with independent enrichment of CHIP-sCNV in microglia, and allowed analysis of the transcriptional effects of CHIP-sCNV in microglia, by creating an integrated snRNAseq atlas of microglia-CAMs identified across Alzheimer's disease cases and controls (FIG. 11) and identifying differentially expressed genes (DEGs) between mutant and wild-type microglia-CAMs from CHIP-sCNV-carrying Alzheimer's disease brains (FIG. 5D). Using gene ontology (GO) enrichment analysis, it was found that DEGs with increased expression in mutant microglia were enriched (adjusted p<0.05, hypergeometric test) for several terms related to immune activation and signaling, suggesting that mutant microglia may upregulate pro-inflammatory pathways (FIG. 5E).

A recent study used human stem-cell differentiated microglia to identify transcriptional signatures of microglial states that emerge in response to various CNS challenges, such as apoptotic neurons, amyloid-beta fibrils, and myelin debris (Dolan, M.-J. et al. bioRxiv (2022)). These signatures were used to further characterize the microglial state associated with CHIP-sCNVs. Using a hypergeometric test for enrichment, marginally significant overlap was found between DEGs that are upregulated in mutant microglia and genes associated with the disease-associated microglia (DAM) state (FIG. 5F; p=0.04). DAMs are specifically enriched in Alzheimer's disease brains and have been posited to play a role in modulating the neuroinflammatory response to neurodegeneration, indicating that microglia with CHIP-sCNV may share a similar phenotype in Alzheimer's disease. Of note, enrichment for a gene set associated with proliferation (p=1) was not found, a microglial state commonly induced in response to infection and tumor, indicating that CHIP-sCNVs induces a signature more specific to neurodegeneration, rather than other brain pathologies.

In summary, the results from three independent Alzheimer's disease cohorts, using three orthogonal approaches, revealed a significant and consistent greater burden of somatic mutations in Alzheimer's disease cerebral cortex samples when compared to matched controls, indicating that brain somatic mutation is associated with Alzheimer's disease. These somatic mutations were enriched in proliferation-related genes that are implicated in cancer, with higher mutant allele fractions in Alzheimer's disease brains, indicating roles in clonal expansion of mutant cell clones. This was also supported by the enrichment of Alzheimer's disease cases with multiple CHIP sSNVs. Many mutations were confirmed to be specifically present in microglia, and potentially CAMs. Finally, using snRNAseq analysis it was found that microglia carrying CHIP mutations showed a pro-inflammatory and disease-associated transcriptional signature compared to wild-type counterparts. The DAM-related signature associated with CHIP-sCNV resembles effects of CHIP mutations in blood myeloid cells that increase the risk of myocardial infarction and stroke while activating immune cascades including IL1β, IL6, and others. These similarities suggest analogous roles of microglial mutations in AD that would likely promote neuronal degeneration.

Two recent studies correlating CHIP mutations in blood with AD risk found no effect (Kessler, M. D. et al. Nature 612, 301-309 (2022)) or a surprising protective effect of blood CHIP on Alzheimer's disease (Bouzid, H. et al. medRxiv (2021)). The varying results highlight the complexity and limitations of our current understanding of the relationship between myeloid cells and microglia. Bouzid et al. and the present examples both found that microglial CHIP somatic mutations were typically shared in the blood of the same individual, as did a small earlier study that also found CHIP-like mutations in Alzheimer's disease brain (Keogh, M. J. et al. Nat Commun 9, 4257 (2018)). Since somatic mutations that lead to blood cancer, when dated by lineage analysis, often arise before birth (Williams, N. et al. Nature 602, 162-168 (2022)), mutation-driven microglial clonal expansion mutations may occur in early progenitors of microglial and blood lineages. Under this assumption, microglia carrying the same CHIP mutations may clonally expand in brain independently from blood. Alternatively, recent studies show that myeloid cells from blood can enter the brain when there is dysfunction of the blood-brain barrier (BBB), an early feature of Alzheimer's disease (Montagne, A. et al. Nature 581, 71-76 (2020)), and can differentiate into microglia-like cells (Marchetti, L. & Engelhardt, B. Vasc Biol 2, H1-H18 (2020)). Others have reported that monocytes can enter the brain and form microglia-like cells even independent of BBB disruption (Mildner, A. et al. Nat Neurosci 10, 1544-1553 (2007); Lund, H. et al. Nat Commun 9, 4845 (2018)). Thus, BBB changes may be a critical feature that might promote access of mutant myeloid cells to the CNS. Conversely, activated microglia can form perivascular clusters in neurodegeneration as a result of BBB breakdown (Davalos, D. et al. Nat Commun 3, 1227 (2012); Ryu, J. K. et al. Nat Immunol 19, 1212-1223 (2018)) which might allow mutant brain microglial cells access to enter the bloodstream.

The above results suggest that microglia are the major cell type carrying the CHIP-like sSNV. Although the FANS results cannot completely exclude CAMs also carrying these somatic mutations, the CSF1R+ cell population contained 3% and 9% CAMs in Alzheimer's disease and control brains, respectively (FIG. 4A), and 5 of the 11 somatic mutations represented >10% cell fractions in the sorted microglial nuclei of Alzheimer's disease brains, including the TET2 p.Pro1194Ser variants with >40% cell fraction. This high mutant allele fraction seems inconsistent with the mutation being limited to blood-derived macrophages even assuming all CAMs came from the blood myeloid lineage.

The above examples highlighted five hotspot genes as well as the PI3K-PKB/Akt pathway (including a PIK3CA p.His1047Leu activating mutation and three loss-of-function mutations in TSC1/2) that were recurrently disrupted by somatic mutations in Alzheimer's disease brains. Drugs targeting such genes could serve as potential therapeutic agents to suppress somatic-mutation-activated microglia and ultimately neurodegeneration in Alzheimer's disease. Since the role of disease-associated microglia in neuronal loss and dysfunction may be a common feature shared across many neurodegenerative diseases as well as in age-associated cognitive decline, studying somatic mutation in Alzheimer's disease may provide an important new approach to understanding the pathogenic mechanisms of dementia and other neurodegenerative conditions. The results described herein above were carried out using the following methods and materials.

### Sample Information

The present examples involve samples and sequencing data from three large-scale Alzheimer's disease (AD) studies, ROSMAP, MayoRNAseq, and SEA-AD. The ROSMAP study consists of two prospective studies of aging, The Religious Order Study (ROS) and the Memory and Aging Project (MAP), in which the participants were enrolled by the Rush Alzheimer's Disease Center with detailed cognitive and neuroimaging phenotyping as well as structured neuropathologic examination during the autopsy at the time of death. The MayoRNAseq study performed detailed clinical phenotyping and multi-omic profiling for 278 participants collected by the Mayo Clinic Brain Bank and Banner Sun Health Research Institute. The SEA-AD study performed single-cell multi-omics, quantitative neuropathology, and deep clinical phenotyping on post-mortem brain tissue from 84 aged donors and 5 additional younger neurotypical controls collected at the University of Washington BioRepository and Integrated Neuropathology laboratory and Precision Neuropathology core. Postmortem samples in all studies were collected and de-identified following the protocol of the corresponding Institutional Review Board with informed consent. The diagnosis of Alzheimer's disease was based on the consensus conclusion from all postmortem data generated by neurologists with expertise in dementia and neurodegeneration.

The RNA-seq bam file and the vcf file of germline mutation calls from matched whole-genome sequencing data generated by the ROSMAP and MayoRNAseq studies were downloaded from the AMP-AD Knowledge Portal (adknowledgeportal.synapse.org/), along with the detailed demographic and clinical information for each sample. The raw single-nucleus RNA sequencing (snRNAseq).h5 matrices for SEA-AD and corresponding clinical and technical metadata were also downloaded from AMP-AD Knowledge Portal. Table 2 and 6 summarized all the bulk and single-nucleus brain RNA-seq samples analyzed for somatic mutation calling. The ROSMAP dataset consists of the prefrontal cortex (PFC) samples of 228 AD patients and 338 age-matched controls with no or mild cognitive impairment collected by the ROSMAP project. The MayoRNAseq dataset consists of the temporal cortex and cerebellum samples from 92 AD patients and 82 age-matched controls collected by Mayo Clinic, most of whom have RNA-seq from both the temporal cortex and cerebellum samples. The SEA-AD dataset consists of the middle temporal gyrus of temporal cortex from 31 AD patients and 32 age-matched controls. In each dataset, the AD and control samples showed similar distributions in sex, age, post-mortem interval, and sequencing depth (Table 2 and 6).

In addition to access to the sequencing data, genomic DNA (gDNA) was obtained from 190 AD patients and 123 controls without cognitive impairment from ROSMAP for panel sequencing (Table 5), though this donor list has minimal overlap with the donor list of the brain RNA-seq dataset due to the limited sample availability. Additional dorsolateral PFC brain samples and gDNA from peripheral blood samples were also obtained from ROSMAP to confirm the presence of somatic mutation and further study the cell type identity of mutation-carrying cells.

### Design of RNA-MosaicHunter

Compared to DNA-seq data, RNA-seq data had unique features that needed to be addressed for somatic mutation calling. First, the exon-intron structure in mRNA required the spliced alignment of RNA-seq reads onto the human reference genome, which increased the chance of alignment errors when the overhang sequence is relatively short (P. G. Engstrom et al., Systematic evaluation of spliced alignment programs for RNA-seq data. Nat Methods 10, 1185-1191 (2013)). Second, the widespread adenosine-to-inosine (A-to-I) RNA editing sites across the human genome are indistinguishable from A-to-G somatic mutations in RNA-seq data, because inosine will be recognized as guanine (G) in Illumina sequencers. Third, the allele-specific expression, a phenomenon that the paternal and maternal alleles have different expression levels, is observed in many autosomal and X chromosome genes, which can lead to deviated allele fraction estimation in RNA-seq data.

To address these technical issues, RNA-MosaicHunter was developed, which was derived from MosaicHunter (A. Y. Huang et al., Postzygotic single-nucleotide mosaicisms in whole-genome sequences of clinically unremarkable individuals. Cell Res 24, 1311-1327 (2014); A. Y. Huang et al., MosaicHunter: accurate detection of postzygotic single-nucleotide mosaicism through next-generation sequencing of unpaired, trio, and paired samples. Nucleic Acids Res 45, e76 (2017)), a bioinformatic tool designed to identify somatic mutations in DNA-seq data. RNA-MosaicHunter consisted of two major components, a Bayesian genotyper to identify real mutations from base-calling errors, followed by a series of empirical error filters to remove artifacts introduced from various sources (FIG. 1A). In the Bayesian genotyper, G denotes the genotype state, π denotes the prior probability of each genotype inferred from the population mutant allele fraction (palt) and default genome-wide somatic mutation rate (pm), and d, q, and o denote the depth, base qualities, and bases for calculating genotype likelihoods from the observed sequencing data. Since the mutant allele fraction in RNA-seq data can be affected by allele-specific expression, the posterior probability of both germline heterozygous mutation and somatic mutation was considered in the list of mutation candidates for subsequent error filters, and further distinguished somatic mutations from germline heterozygous mutations by using the genotyping results from matched whole-genome or whole-exome sequencing data obtained from the same individual. In addition, RNA-MosaicHunter also incorporated other filters to exclude 1) candidates with less than 5% mutation allele fraction or less than 5 mutant-supporting reads; 2) candidates that are in repetitive and homopolymer regions; 3) candidates that have a significant bias in strand, mapping quality, or within-read position between the reference and mutant alleles; 4) candidates that show complete linkage to adjacent candidates on the same read or read pairs, which is more likely to be caused by alignment errors; 5) candidates that are supported by more than 50% of the “high-quality” reads after confirming the alignment by a second aligner and masking bases adjacent to the start, end or spliced junctions of each read; and 6) candidates that are recurrently present in the RNA-seq data of more than two unrelated individuals. The source code and default configuration file of RNA-MosaicHunter are publicly available at gitlab.aleelab.net/august/ma-mosaichunter.git, and it supports users to customize parameters that are used in the Bayesian genotyper and empirical error filters.

Somatic Mutation Calling from RNA-Seq Data

Each downloaded RNA-seq bam file was first converted back to the fastq format by Picard (v1.138) and then aligned to the GRCh37 human reference genome by STAR (v2.5.0a) (A. Dobin et al., STAR: ultrafast universal RNA-seq aligner. Bioinformatics 29, 15-21 (2013)) in the two-pass mode, where the reference gene annotation (Gencode version 19) was used in the first pass and then a sample-specific annotation generated from the first pass was used in the second pass. The aligned reads were processed by Picard (v1.138) to remove duplicates, followed by SplitNCigarReads, indel realignment, and base quality recalibration of GATK (v3.6) (M. A. DePristo et al., A framework for variation discovery and genotyping using next-generation DNA sequencing data. Nat Genet 43, 491-498 (2011)). Reads that were improperly paired or with ambiguous alignment were removed, and only genomic positions covered by 10 or more reads were subject to RNA-MosaicHunter. To exclude A-to-I(G) RNA editing sites, only non-A-to-G candidates were considered from the output of RNA-MosaicHunter. Non-exonic candidates and candidates that are present in the polymorphism databases of the general human population were excluded including dbSNP (S. T. Sherry et al., dbSNP: the NCBI database of genetic variation. Nucleic Acids Res 29, 308-311 (2001)), the 1000 Genomes Project (C. Genomes Project et al., An integrated map of genetic variation from 1,092 human genomes. Nature 491, 56-65 (2012)), the Exome Sequencing Project (J. A. Tennessen et al., Evolution and functional impact of rare coding variation from deep sequencing of human exomes. Science 337, 64-69 (2012)), and the Exome Aggregation Consortium (M. Lek et al., Analysis of protein-coding genetic variation in 60,706 humans. Nature 536, 285-291 (2016)).

### Benchmarking of RNA-MosaicHunter

RNA-seq and whole-exome sequencing data of 19 esophageal carcinoma samples as well as whole-exome sequencing data of their matched normal samples were downloaded from The Cancer Genome Atlas (TCGA) Research Network (N. Cancer Genome Atlas Research et al., Integrated genomic characterization of oesophageal carcinoma. Nature 541, 169-175 (2017)). The list of 19 esophageal carcinoma samples is shown below: TCGA-L5-A40F-01A, TCGA-V5-A7RC-01B, TCGA-LN-A4A1-01A, TCGA-IG-A97I-01A, TCGA-L5-A8NE-01A, TCGA-JY-A93C-01A, TCGA-LN-A49M-01A, TCGA-IG-A3YB-01A, TCGA-LN-A49Y-01A, TCGA-L5-A8NN-01A, TCGA-LN-A49L-01A, TCGA-LN-A9FQ-01A, TCGA-L5-A40R-01A, TCGA-LN-A811-01A, TCGA-L5-A891-01A, TCGA-L7-A6VZ-01A, TCGA-LN-A4A4-01A, TCGA-LN-A5U5-01A, TCGA-L5-A40J-01A.

Somatic mutation calls created by the Broad Institute through the comparison of tumor and matched normal whole-exome sequencing pairs using MuTect (K. Cibulskis et al., Sensitive detection of somatic point mutations in impure and heterogeneous cancer samples. Nat Biotechnol 31, 213-219 (2013)) were also downloaded. A total of 851 non-A-to-G, autosomal, exonic, tumor-specific somatic mutations were called from the 19 tumor samples and covered by 10 or more reads in tumor RNA-seq data. This call-set served as the gold standard for benchmarking the RNA-seq somatic mutation calling pipeline. The calling pipeline was applied to 19 esophageal tumor RNA-seq profiles, without applying a filter for removing recurrent candidates because these tumor samples may share common driver mutations, and identified 613 non-A-to-G somatic mutations.

By comparing the RNA-MosaicHunter callset with the gold standard, RNA-MosaicHunter was found to have successfully identified 499 out of 851 MuTect-called mutations, equivalent to a sensitivity of 59% (FIG. 2B). On the other hand, among 613 RNA-MosaicHunter-called mutations, 513 were confirmed by the MuTect calls while 65 mutations were missed by MuTect but showed reads with 2% or more mutant allele fractions in the DNA-seq data, suggesting an overall precision of 94% for RNA-MosaicHunter (FIGS. 2A-2B).

### Neuronal Proportion Estimation

To estimate the proportion of neurons and other brain cell types in bulk brain RNA-seq data of ROSMAP and MayoRNAseq, CIBERSORT (v1.05) (H. Li, R. Durbin, Fast and accurate short read alignment with Burrows-Wheeler transform. Bioinformatics 25, 1754-1760 (2009)) was applied to deconvolute the cell-type composition for each RNA-seq sample, by using the cell-type-specific expression reference for different neuronal and glial types (excitatory and inhibitory neuronal subtypes in the cortex, cerebellar granule cells, Purkinje cells, endothelial cells, pericytes, astrocytes, oligodendrocytes and their precursor cells, and microglia), generated from a large-scale brain single-cell RNA-seq dataset (T. Dunn et al., Pisces: an accurate and versatile variant caller for somatic and germline next-generation sequencing data. Bioinformatics 35, 1579-1581 (2019)). The estimated proportion of all subtypes of excitatory and inhibitory neurons were summed to calculate the overall neuronal proportion for each sample.

### Panel Design and Sequencing

Probes targeting the exons and exon-intron junctions of 149 proliferation-related genes (Table 4) were designed using the SureSelect DNA Advanced Design Wizard. The list of targeted genes was designed to include frequently mutated oncogenes and tumor suppressor genes in various types of cancer and clonal hematopoiesis. A total of 23,171 probes with a genomic size of 691 kbp were designed and generated. These probes were then used for gene capture followed by library preparation using the SureSelect XT HS2 DNA Reagent Kit with 30 ng gDNA input. All prepared libraries were sequenced using three Illumina NovaSeq 6000 S4 flow cells with 150 bp paired-end reads.

Somatic Mutation Calling from Panel Sequencing

The unique molecular identifier information of each read was first extracted from the fastq files by AGeNT's Trimmer (v2.0.2), and then reads were aligned to the GRCh37 human reference genome by BWA-MEM (v0.7.15) (H. Li, R. Durbin, Fast and accurate short read alignment with Burrows-Wheeler transform. Bioinformatics 25, 1754-1760 (2009)). The aligned reads were processed by AGeNT LocatIt (v2.0.2) to generate the consensus read sequence from multiple reads that were derived from the same original DNA fragment and thus carried the same unique molecular identifier, followed by GATK's indel realignment (v3.6) (M. A. DePristo et al., A framework for variation discovery and genotyping using next-generation DNA sequencing data. Nat Genet 43, 491-498 (2011)). Only the consensus reads were kept that were supported by two or more reads in both strands. As a result, comparable depth and coverage was achieved between the Alzheimer's disease and control samples, with more than 1000× average depth and more than 80% coverage of the targeted regions at >500× for consensus reads (Table 5; FIG. 7A).

Somatic SNVs and indels were called from the consensus reads by MosaicHunter (v1.0) (A. Y. Huang et al., MosaicHunter: accurate detection of postzygotic single-nucleotide mosaicism through next-generation sequencing of unpaired, trio, and paired samples. Nucleic Acids Res 45, e76 (2017)) and Pisces (v5.3) (T. Dunn et al., Pisces: an accurate and versatile variant caller for somatic and germline next-generation sequencing data. Bioinformatics 35, 1579-1581 (2019)), respectively. For somatic SNVs, MosaicHunter calculated the likelihood of the presence of a mutant allele, and only the candidates with a 0.5 or higher likelihood, 100 or more total reads, and 4 or more mutant-supporting reads were considered. Candidates as germline mutations were excluded if i) they had a 30% or higher mutation allele fraction; 2) the counts of mutant-supporting and total reads did not significantly deviate from the binomial distribution for heterozygous mutations (p>0.05); or 3) they were present in the polymorphism databases (dbSNP (S. T. Sherry et al., dbSNP: the NCBI database of genetic variation. Nucleic Acids Res 29, 308-311 (2001)), the 1000 Genomes Project (C. Genomes Project et al., An integrated map of genetic variation from 1,092 human genomes. Nature 491, 56-65 (2012)), the Exome Sequencing Project (J. A. Tennessen et al., Evolution and functional impact of rare coding variation from deep sequencing of human exomes. Science 337, 64-69 (2012)), and the Exome Aggregation Consortium (M. Lek et al., Analysis of protein-coding genetic variation in 60,706 humans. Nature 536, 285-291 (2016))) or had a 0.010% or higher population allele frequency in the Genome Aggregation Database (K. J. Karczewski et al., The mutational constraint spectrum quantified from variation in 141,456 humans. Nature 581, 434-443 (2020)). Somatic indels were called by Pisces with its default parameters, and a similar method was used to call mutation candidates and remove germline mutations.

To balance the sensitivity and specificity of the somatic SNV and indel detection, two different pipelines were developed when considering the recurrent presence across multiple individuals. The “stringent” pipeline only kept the mutations that were detected in one sample and completely absent in any other samples, whereas the “sensitive” pipeline additionally allowed the mutations that were exclusively present or specifically enriched (two-sample Z-test of proportion with p<0.05) in the Alzheimer's disease or control group.

### Benchmarking of Mutation Calling Using Panel Sequencing

A mixing experiment was performed for benchmarking the performance of the designed panel and variant calling pipeline. Germline mutation calls from two unrelated individuals, NA12878 and NA24695, were downloaded from the website of the Genome in a Bottle Consortium (J. M. Zook et al., An open resource for accurately benchmarking small variant and reference calls. Nat Biotechnol 37, 561-566 (2019)). Genomic sites in the covered regions of panel sequencing that were genotyped as heterozygous in NA24695 but reference-homozygous in NA12878 were considered as the gold-standard list of somatic mutations, and gDNA from these two individuals were mixed to reach 10%, 5%, 2%, 1%, 0.5% and 0.2% mutant allele fractions for these mutations. The same experiment and analysis protocols of panel sequencing were applied to the mixed samples with varied allele fractions, and then the proportion of gold-standard mutations were checked that were identified by the identification pipeline as well as the consistency between expected and observed allele fractions.

### Fluorescence-Activated Nuclei Sorting (FANS)

Nuclei were prepared following the previously published work (M. B. Miller et al., Somatic genomic changes in single Alzheimer's disease neurons. Nature 604, 714-722 (2022)). Briefly, fresh frozen human brain tissue samples were first lysed in a dounce homogenizer using a chilled nuclear lysis buffer (10 mM Tris-HCl, 0.32 M Sucrose, 3 mM Mg(Acetate)₂, 5 mM CaCl₂), 0.1 mM EDTA, pH 8, 1 mM DTT, 0.1% Triton X-100) on ice. Tissue lysates were layered on top of a sucrose cushion buffer (1.8 M sucrose 3 mM Mg(OAc)₂, 10 mM Tris-HCl, 1 mM DTT, pH 8) and ultra-centrifuged for 1 h at 30,000 g. Nuclear pellets we resuspended in ice-cold PBS supplemented with 3 mM MgCl2, filtered, then stained with the neuronal marker (NeuN, Millipore MAB377) or microglial marker (CSF1R, Cell Signaling 65396) together with DAPI. For each brain sample, neuronal (NeuN+), glial (NeuN-), microglial (CSF1R+) and total (DAPI+) nuclei populations were sorted into 96-well plates by flow cytometry.

10× Single-Nuclei RNA-Seq (snRNAseq)

For the prefrontal cortex (PFC) sample of one Alzheimer's disease patient (with a TET2 p.Pro1194Ser sSNV) and one healthy control, ten thousand microglial nuclei were sorted separately into a well of the 96-well plate and used for droplet generation and sequencing library preparation using the 10× Genomics Next GEM Single Cell 3′ GEM Kit v3.1 and Chromium Controller, following the manufacturer's manual. The snRNAseq libraries were sequenced by Illumina HiSeq X, and down-sampled to have a comparable sequencing throughput. The sequencing data of each sample was first processed by Cell Ranger (v6.0.0) (G. X. Y. Zheng et al., Massively parallel digital transcriptional profiling of single cells. Nature Communications 8, 14049 (2017)) and Pagoda2 (v0.1.0) (J. Fan et al., Characterizing transcriptional heterogeneity through pathway and gene set overdispersion analysis. Nature Methods 13, 241-244 (2016)) separately, and then integrated by Conos (v1.4.6) (N. Barkas et al., Joint analysis of heterogeneous single-cell RNA-seq dataset collections. Nat Methods 16, 695-698 (2019)), for variance normalization, clustering with the Leiden method, and large Vis embedding and visualization. Cell clusters were manually annotated into different cell types based on the expression profile of marker genes (FIG. 9B) for the major neural (A. Y. Huang et al., Parallel RNA and DNA analysis after deep sequencing (PRDD-seq) reveals cell type-specific lineage patterns in human brain. Proc Natl Acad Sci USA 117, 13886-13895 (2020)) and blood (M. Olah et al., Single cell RNA sequencing of human microglia uncovers a subset associated with Alzheimer's disease. Nat Commun 11, 6129 (2020)) cell types (HBA1: red blood cell; CD3E: T-cell; CCR7: B-cell; FCN1: monocyte). The snRNAseq results confirmed 77-79% microglia purity in the CSF1R+ sorted nuclei of the Alzheimer's disease and control brains, with additional 3-9% CNS-associated macrophages (FIG. 4A). Minimal blood contamination was observed in the sorted microglial population, with only 1% monocytes and the absence of other major blood cell types including red blood cells, T-cells, and B-cells (FIG. 4A and FIG. 9B).

To estimate the proportion of blood cells in unsorted bulk brain samples of ROSMAP, a large-scale snRNAseq dataset was downloaded, consisting of 80,660 nuclei isolated from 24 Alzheimer's disease and 24 control PFC samples collected by ROSMAP. The expression matrix of ten thousand randomly-selected high-quality nuclei (with ≥100 detected genes) was extracted and then analyzed by Pagoda2 (v0.1.0) by a similar protocol as described above. No clusters were observed that expressed marker genes of any major blood cell types, which confirmed the minimal contamination of blood cells in ROSMAP brain samples.

### Amplicon Sequencing

Amplicon sequencing was performed for validation and mutant allele fraction estimation in both bulk gDNA samples and sorted nuclei. Bulk gDNA was extracted from frozen brain samples using the EZ1 DNA Tissue Kit (Qiagen 953034). Five hundred nuclei of each cell type from each brain sample were sorted into 96-well plates with four replicates. Whole-genome amplification was then performed for sorted nuclei using the ResolveDNA Whole Genome Amplification Kit (BioSkryb Genomics) to meet the minimal DNA amount for panel sequencing. For each identified sSNV, three sets of primers were designed for PCR amplification of the targeted genomic region. PCR amplification was performed using the Phusion Hot Start II DNA Polymerase kit (Thermo Fisher F549L) with the following cycles: 98° C. for 30 sec; 5 cycles of 98° C. for 10 sec, 68° C. for 30 sec (decrease 1° C./cycle), and 72° C. for 30 sec; 25 cycles of 98° C. for 10 sec, 63° C. for 30 sec, 72° C. for 30 sec; 72° C. for 10 min. The annealing temperatures of primers varied for each design which was determined by a testing PCR. PCR products were then purified using AMPure XP beads (Beckman Coulter A63882) and pooled for Amplicon-EZ sequencing (GENEWIZ).

The sequencing reads were first aligned to the GRCh37 human reference genome by BWA-MEM (v0.7.15) (H. Li, R. Durbin, Fast and accurate short read alignment with Burrows-Wheeler transform. Bioinformatics 25, 1754-1760 (2009)) and then processed by GATK (v3.6) for indel realignment (M. A. DePristo et al., A framework for variation discovery and genotyping using next-generation DNA sequencing data. Nat Genet 43, 491-498 (2011)). For each somatic mutation candidate, the number of reads supporting each allele was calculated by MosaicHunter (v1.0) and manually verified by Integrative Genomics Viewer (v2.3.93) (J. T. Robinson et al., Integrative genomics viewer. Nat Biotechnol 29, 24-26 (2011)). A candidate was considered validated as a somatic mutation (FIGS. 7E-7G) if 1) the read fraction of the mutant allele was more than three times as high as the fractions of the other two error alleles in all three amplicons (somatic-I); or 2) the read fraction of the mutant allele in the corresponding brain sample was significantly higher than the fraction in an unrelated negative control brain sample for all three amplicons (somatic-II).

### Functional Annotation of Somatic Mutations

ANNOVAR (v2015Mar22) (K. Wang, M. Li, H. Hakonarson, ANNOVAR: functional annotation of genetic variants from high-throughput sequencing data. Nucleic Acids Res 38, e 164 (2010)) was applied to annotate somatic mutations into different genic categories: 5′ UTR, exonic (coding sequence), 3′ UTR, splicing (within intronic 2 bp of a splicing junction), and intronic. Exonic somatic mutations were further classified into multiple categories based on their predicted impacts on amino acids. A somatic mutation was labeled as deleterious if 1) it was annotated as splicing or predicted to cause stop-codon gain/loss; 2) it was a frameshift insertion or deletion; or 3) it was a missense mutation whose amino acid change was predicted to be deleterious by either PolyPhen2 (I. A. Adzhubei et al., A method and server for predicting damaging missense mutations. Nat Methods 7, 248-249 (2010)) or SIFT (P. Kumar, S. Henikoff, P. C. Ng, Predicting the effects of coding non-synonymous variants on protein function using the SIFT algorithm. Nat Protoc 4, 1073-1081 (2009)). For 149 proliferation-related genes, oncogenes and tumor suppressor genes (TSGs) were grouped according to the annotation of the COSMIC Cancer Gene Census (Z. Sondka et al., The COSMIC Cancer Gene Census: describing genetic dysfunction across all human cancers. Nat Rev Cancer 18, 696-705 (2018)). Genes annotated as both oncogenes and tumor suppressor genes were not considered in calculating the mutation burdens plotted in FIG. 3D. MAFTools (v2.10.1) (A. Mayakonda, D. C. Lin, Y. Assenov, C. Plass, H. P. Koeffler, Maftools: efficient and comprehensive analysis of somatic variants in cancer. Genome Res 28, 1747-1756 (2018)) was used to illustrate the gene-level distribution of somatic mutations. Genes and driver mutations involved in clonal hematopoiesis of indeterminate potential (CHIP) were extracted from a study that analyzed blood whole-genome sequencing data from 11,262 people (F. Zink et al., Clonal hematopoiesis, with and without candidate driver mutations, is common in the elderly. Blood 130, 742-752 (2017)).

Functional enrichment analysis of Gene Ontology (GO) terms was performed using GOseq (v1.34.1) (M. D. Young, M. J. Wakefield, G. K. Smyth, A. Oshlack, Gene ontology analysis for RNA-seq: accounting for selection bias. Genome Biol 11, R14 (2010)). Exonic somatic mutations identified from the RNA-seq of Alzheimer's disease patients or normal controls were used as the input, and the Wallenius approximation method was used to test the enrichment, with a probability weighting function to control for potential gene length bias. Only GO terms with 3 or more hits and an initial overrepresentation p-value <0.01 were considered. GO terms with more than 1000 genes were excluded. All the GO terms with significant enrichment of Alzheimer's disease somatic mutations were plotted in FIG. 2F, where the p-value was adjusted by Hommel's method for the correction of multiple hypothesis testing. In comparison, only one GO term “helicase activity” showed significant enrichment for somatic mutations identified from normal controls.

### Burden Analysis of Somatic Mutations

Somatic mutation density in each clinical group was calculated by counting the total number of somatic mutations and dividing it by the total size of powered genomic regions with ≥10× coverage for RNA-seq or ≥500× for panel sequencing data sets, and the odds ratio and the two-sample Z-test of proportion was used to test whether the Alzheimer's disease group had a higher mutation burden than the control group. In the gene-level analysis for panel sequencing data, the somatic mutation burden was compared between Alzheimer's disease and control groups using a similar two-sample Z-test of proportion, in which the total genomic size for each gene was calculated as the product of the exonic length and the number of individuals in Alzheimer's disease or control group.

For the linear regression analysis, the count of somatic mutations in each sample was modeled as a continuous outcome, whereas clinical status and other covariates of interest (e.g., age, gender, sequencing depth, post-mortem interval, and neuronal proportion) were modeled as independent variables. The linear regression results from both RNA-seq and panel sequencing confirmed the increased burden of somatic mutation in Alzheimer's disease brains after controlling for all of these potential confounding factors (FIGS. 2E and 3C). Only donors with ages less than 90 were considered, because all the donors with age 90 or higher were labeled as “90+” in the demographic tables of the ROSMAP and MayoRNAseq studies. Whether Alzheimer's disease patients with different APOE alleles showed different somatic mutation burdens were tested, but a statistical significance was not observed. To further rule out the effect of potential blood contamination, the normalized gene expression level (transcript per million, TPM) of blood marker genes was measured including HBA1, CD3E, CCR7, and FCN1 for each RNA-seq sample of ROSMAP and MayoRNAseq by StringTie (v1.3.3b) (M. Pertea et al., StringTie enables improved reconstruction of a transcriptome from RNA-seq reads. Nat Biotechnol 33, 290-295 (2015)). These were then modeled as additional covariates in the linear regression model. Minimal contamination of blood-derived immune cells was observed in ROSMAP and MayoRNAseq brain samples, and the observed Alzheimer's disease increase remained significant after controlling for any of these genes (p<0.01).

Automatic Cell-Type Identification with scType

Myeloid cells in the brain include both parenchymal microglia and CNS-associated macrophages (CAMs), including meningeal, choroid plexus, and perivascular macrophages (PVMs). Microglia-perivascular macrophages, hereby referred to as microglia-CAMs, represented 3.37% of all pre-annotated cells within SEA-AD. scType (v20220909) (Ianevski, A., Girn, A. K. & Aittokallio, T. Nat Commun 13, 1246 (2022)) was used to automatically identify any additional high-quality microglia-CAMs beyond those originally annotated in SEA-AD (“pre-annotated” cells) to increase statistical power for calling somatic copy number variants (sCNVs). Excitatory neurons (ExNs) were also automatically typed as a cell-type out-group to further facilitate accurate identification of microglia-CAMs, as scType'd microglia-CAMs should have high microglia-CAM scType scores but low ExN scType scores.

Prior to running scType, each SEA-AD sample was processed, normalized, and clustered with the Louvain algorithm using Seurat (v4.1.1) (Hao, Y. et al. Cell 184, 3573-3587 e3529 (2021)). Each sample underwent quality control with the following metrics: retain only 1) genes expressed in ≥3 cells, 2) cells with ≥10 expressed genes, 3) cells with ≤5% mitochondrial gene expression, 4) cells with >250 expressed genes and <7500 expressed genes. Positive markers for microglia-CAMs (P2RY12, ITGAM, CD40, PTPRC, CD68, AIF1, CX3CR1, TMEM119, ADGRE1, C1QA, NOS2, TNF, ISYNA1, CCL4, ADORA3, ADRB2, BHLHE41, BIN1, KLF2, NAV3, RHOB, SALL1, SIGLEC8, SLCIA3, SPRY1, TAL1) and ExNs (SLCI7A7, SLCI7A6, GRIN1, GRIN2B, GLS, GLUL, GRIN2A) were downloaded from the scType marker database and used to calculate microglia-CAM and ExN scType scores for each individual cell.

In brief, scType calculates cell-type specific scores for each cell using a weighted and normalized aggregation of marker gene expression, where marker genes are weighted more highly if they are more specific for a given cell type (expressed in one cell type of interest, rather than several). For each sample, both ExN and microglia-CAM scType scores were calculated for cells that were pre-annotated as either ExNs or microglia-CAMs. Taking these pre-annotations as ground truth, ROCR (v1.0.11) (Sing, T. et al., Bioinformatics 21, 3940-3941 (2005)) and cutpointr (v1.1.2) (Thiele, C. & Hirschfeld, G. J Stat Softw 98 (2021)) were used to calculate the optimal cutpoint for ExN and microglia-CAM scType scores that maximized the sum of sensitivity and specificity of classification over 1000 bootstraps. Using these learned ExN and microglia-CAM cutpoints, cells that were not pre-annotated were assigned as ExNs, microglia-CAMs, or neither. A small number of cells had both microglia-CAM and ExN scType scores greater than the corresponding optimal cutpoints; these cells were discarded due to ambiguity in assignment.

In addition to filtering of individual cells, 6 samples were filtered out due to not meeting at least one of the following sample-specific metrics: 1) microglia-CAM AUC>0.9, 2) ExN AUC>0.9, 3) fraction of pre-annotated ExN typed by scType as microglia <0.1, and 4) total number of pre-annotated and scType'd microglia-CAMs>50. This analysis filtered one individual H20.33.008, as this donor had only one associated sample that was filtered due to not meeting the above sample-specific metrics.

As a final step to ensure that scType'd cell microglia-CAMs were highly similar to their corresponding pre-annotated cell types, pre-annotated and scType'd microglia-CAMs derived from the same donor were merged into a single Seurat object and processed, normalized, and clustered using the Louvain algorithm. Clusters in which over 50% of cells were pre-annotated microglia-CAMs were identified and only scType'd microglia-CAMs in these clusters were retained as high-confidence scType'd microglia-CAMs cells. Only pre-annotated microglia-CAMs and these high-confidence scType'd microglia-CAM cells were used for sCNV-calling and all subsequent downstream analyses.

Somatic CNV Calling from snRNAseq

Genomic regions of non-uniparental disomy CHIP-sCNV listed in a previous study (Saiki, R. et al. Nat Med 27, 1239-1249 (2021)) were extracted, and genomic coordinates of these regions were downloaded from the hg38 reference genome accessed through the UCSC Genome Browser (Kent, W. J. et al. Genome Res 12, 996-1006 (2002)).

sCNV calling was done for microglia-CAM, astrocytes, oligodendrocytes, oligodendrocyte precursor cells (OPCs), and ExNs. For each cell type, raw count matrices (gene×cell) were extracted for the 31 AD cases and 31 age-matched healthy controls that passed filtering as described above. Each of these matrices was processed and normalized using Seurat (v4.1.1) (Hao, Y. et al. Cell 184, 3573-3587 e3529 (2021)) and then independently used as input for sCNV-calling with CONICSmat (v0.0.0.1) (Muller, S. et al., Bioinformatics 34, 3217-3219 (2018)).

The aforementioned CHIP-sCNV regions were tested with CONICSmat, and raw CHIP-sCNV calls were further filtered to increase specificity of calls. In brief, a putative CHIP-sCNV was retained if it met the following criteria: 1) Bonferonni adjusted p-value <0.05; 2)<25% ambiguous cells (cells with a posterior probability >0.25 and <0.75); 3) median expression of putative CNV-carrying cells is > or <1.96 standard deviations of putative normal cells of the same type for amplifications or deletions, respectively; 4) no negative control regions (i.e. whole chromosome regions that have not been associated with CHIP-sCNV in past literature) showed a larger difference in expression between putative normal and CNV-carrying cells than the called CNV; 5) the expression of putative normal cells was within 1.96 standard deviations of baseline expression of cells of the same type across all other individuals; and 6) the same CNV was not called in a different cell-type from the same individual. For microglia-CAMs, putative CNVs were additionally filtered if the number of scType'd non-ambiguous cells (posterior probability <0.25 or >0.75) were <1.5× the number of pre-annotated non-ambiguous cells for both altered and wild-type cells. This filtering criterion was added to ensure that CNV calls called from scType'd and pre-annotated microglia-CAMs were not driven by added scType'd cells.

Burden Analysis of sCNV

Per cell type, the number of cells with CNVs from Alzheimer's disease donors, the number of cells without CNVs from Alzheimer's disease donors, the number of cells with CNVs from control donors, and the number of cells without CNVs from control donors were counted and an odds ratio (OR) of CNV-carrying cells in Alzheimer's disease donors vs control donors was calculated. For two cell types, CAMs and oligodendrocytes, all CNV-carrying cells were in Alzheimer's disease donors and the OR was thus infinite. To facilitate comparison of the actual OR against an empirical null as described below, a pseudocount of 1 was added to the number of CNV-carrying cells in Alzheimer's disease and control groups separately for these two cell types. To calculate the significance level of this calculated odds ratio, an empirical null was generated using permutation. In brief, for each cell type, diagnosis labels were permuted over the set of all cells from each donor, including both CNV-carrying and wild-type cells. If a donor had multiple called CNVs, diagnosis labels were permuted over each CNV individually. Specifically, for each called CNV in a given individual, cells were divided into wild-type or CNV-carrying for that specific CNV. Each of these partitions of wild-type versus CNV-carrying cells was then randomly assigned a diagnosis status. OR was calculated for each set of permutated data. Permutations were repeated 1000 times and the p-value of the actual OR was calculated as 1—the percentile rank of actual OR against the empirical null distribution of permutation ORs. Ten trials of 1000 permutations were completed to ensure the robustness of p-values.

Creation of an Integrated snRNAseq Microglia-CAM Atlas

All scType'd and pre-annotated microglia-CAMs from Alzheimer's disease and healthy control samples, with the exception of the one associated with H20.33.008 as described above, were individually processed with Seurat (v4.1.1). In brief, each sample underwent quality control with the following metrics: retain only 1) genes expressed in ≥3 cells, 2) cells with ≥10 expressed genes, 3) cells with <5% mitochondrial gene expression, 4) cells with ≥250 expressed genes and <7500 expressed genes. Variance-stabilizing normalization and regression of the technical covariates percent.mt, nFeature_RNA, and nCount_RNA were performed with Seurat function SCTransform, and clustering was done using the Louvain algorithm.

Individual samples were then merged into a single Seurat object, and dimensionality reduction was performed using principle component analysis (PCA). This merged object was then integrated over constituent individual samples using Seurat's wrapper function for Harmony (v0.1.1) (Korsunsky, I. et al. Nat Methods 16, 1289-1296 (2019). UMAP visualization of the integrated object showed no visible clustering by sample ID or individual ID, consistent with successful integration (FIG. 11).

Differential Expression Analysis and Functional Annotation of Integrated Microglia-CAM snRNAseq Atlas

Differential expression analysis was performed between microglia-CAMs with and without called CNVs from CNV-carrying Alzheimer's disease individuals using the FindMarkers function of Seurat (v4.1.1) with a min.pct cutoff of 0.10 and no fold-change cutoff. Genes with an adjusted p-value <0.05 were called as differentially-expressed genes (DEGs).

clusterProfiler (v4.4.4) (Wu, T. et al. Innovation (Camb) 2, 100141 (2021)) was used to perform all enrichment analyses. GO enrichment analysis was performed using standard parameters and a universe of all genes expressed in >10% of microglia-CAMs in the integrated atlas. Terms were deemed significant if they had an adjusted p-value <0.05.

DEGs were also tested for enrichment of previously-defined microglial state gene modules (Dolan, M.-J. et al. bioRxiv (2022)). A minority of genes (107/905; 11.9%) within these microglial state gene modules were shared between multiple modules. To ensure specificity of module enrichment, genes were weighted by the inverse of the number of modules in which they were present. Non-integer values were rounded and module enrichment was tested using a hypergeometric test.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of characterizing neuroinflammation in a biological sample of a subject, the method comprising:
a) sequencing a set of proliferation-related or cell cycle-related polynucleotides to generate sequencing reads, wherein each polynucleotide comprises a unique molecular identifier (UMIs), and wherein the polynucleotides are derived from a biological sample of a subject comprising somatic cells;
b) generating a consensus sequence for each group of sequencing reads comprising the same UMI;
c) comparing the consensus sequence to a reference sequence to identify somatic single nucleotide variations (sSNVs) and somatic indels in the consensus sequence that are not present in the reference sequence, wherein the presence of sSNVs and somatic indels in the consensus sequence is indicative of neuroinflammation in the biological sample.

2. A computer implemented method of determining a burden of somatic mutations in a biological sample of a subject, the method comprising:
a) sequencing RNA present in a biological sample comprising somatic cells of the subject to generate sequencing data;
b) aligning a set of RNA sequences in the sequencing data against a human reference genome to obtain alignment reads;
c) filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample; and
d) calculating the burden of somatic mutations in the biological sample relative to the reference sequence.

3. The method of claim 2, wherein the method further comprises:
de-convoluting sequencing data and comparing it against a reference set of cell-type specific sequencing data to calculate the proportion of cell types present in the sample;
generating a consensus read sequence from multiple reads derived from an original polynucleotide fragment, wherein each of the multiple reads comprises a UMI that is also present in the original polynucleotide fragment; and wherein consensus reads supported by fewer than two reads on both strands are excluded; and/or
filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample

4. The method of claim 1, wherein the biological sample is derived from a subject having or suspected of having a neuroinflammatory condition selected from the group consisting of Alzheimer's disease, Parkinson's Disease, Huntington's Disease, Fronto-temporal Dementia, Amyotrophic Lateral Sclerosis (ALS), HIV-related dementia, Progressive Supranuclear Palsy (PSP), Pick's disease, Post-COVID encephalopathy, Chronic Traumatic Encephalopathy (CTE), and Lewy Body Disease (LBD).

5. A method of characterizing Alzheimer's disease in a biological sample of a subject, the method comprising:
a) deep DNA panel sequencing a set of proliferation-related or cell cycle-related polynucleotides to generate sequencing reads, wherein each polynucleotide comprises a unique molecular identifier (UMIs), and wherein the polynucleotides are derived from a biological sample comprising somatic cells;
b) generating a consensus sequence for each group of sequencing reads comprising the same UMI;
c) comparing the consensus sequence to a reference sequence to identify somatic single nucleotide variations (sSNVs) and somatic indels in the consensus sequence that are not present in the reference sequence, wherein the presence of sSNVs and somatic indels in the consensus sequence relative to a reference is indicative of Alzheimer's disease or a propensity to develop Alzheimer's disease in the subject and the absence of sSNVs and somatic indels in the consensus sequence relative to a reference is indicative that the subject does not have Alzheimer's disease or a propensity to develop Alzheimer's disease; or
a) sequencing RNA present in a biological sample comprising somatic cells of the subject to generate sequencing data;
b) aligning a set of RNA sequences in the sequencing data against a human reference genome to obtain alignment reads;
c) filtering the alignment reads to remove duplicate genomes, for indel realignment, for base quality recalibration to exclude improperly paired or ambiguous alignments, excluding A-to-I(G) RNA editing sites, such that only non-A-to-G RNA sequences undergo further analysis, and/or excluding non-exonic candidates and candidates that are present in the polymorphism databases of the general human population; thereby generating a set of non-A-to-G, autosomal, exonic, somatic mutations present in the sample; and
d) calculating the burden of somatic mutations in the biological sample relative to the reference sequence, wherein an increase in the burden of somatic mutations is indicative that the subject has or has a propensity to develop Alzheimer's disease; or
a) bulk sequencing RNA from a biological sample of a subject to identify somatic mutations in expressed genes;
b) deep sequencing genomic DNA from a biological sample of the subject to identify somatic single-nucleotide variations in the genomic DNA; and
c) determining the number of somatic single-nucleotide variations identified by bulk sequencing RNA and deep sequencing of genomic DNA relative to a reference, wherein increased somatic single-nucleotide variations in RNA and DNA relative to the reference indicates neuroinflammation or Alzheimer's disease.

6. The method of claim 1, wherein deep sequencing is performed on a polynucleotide comprising a tumor suppressor gene or RNA transcribed from a tumor suppressor gene.

7. The method of claim 1, wherein the sSNV, sIndel, or mutation burden is detected in a gene selected from the group consisting of: AKT3, AKT1, ASXL1, ATRX BCR, CBL; CHEK2, CHK2, CX3CR1, DEPDC5; DNMT3A, EPPK1, KMT2D, MED12, MLH1, MS4A7MRC1, MTOR, PDGFRA, PI3K, PIK3CA, PIK3R1, PIK3R2, PPM1D, P2RY12, STAT3, TSC1, TET2, TMEM119, and TP53.

8. A panel of capture molecules for detection of an sSNV, sIndel, or mutation burden in a gene selected from the group consisting of: AKT3, AKT1, ASXL1, ATRX BCR, CBL; CHEK2, CHK2, CX3CR1, DEPDC5; DNMT3A, EPPK1, KMT2D, MED12, MLH1, MS4A7 MRC1, MTOR, PDGFRA, PI3K, PIK3CA, PIK3R1, PPM1D, P2RY12, STAT3, TSC1, TET2, TMEM119, and TP53, wherein the capture molecule is a polynucleotide or polypeptide.

9. The panel of claim 8, the panel comprising capture molecules each of which specifically binds one or more of the following genes:
TET2, ASXL1, KMT2D, ATRX and CBL;
CX3CR1, TMEM119, and P2RY12;
MS4A7 and MRC1;
DNMT3A and TET2;
DNMT3A (C.1429+1G>A), PPM1D (Leu484fs), TET2 (Val1371Asp), CBL (Leu380Pro), TET2 (Pro1194Ser), TP53 (Arg280Gly), FGFR1 (Arg473Gln);
PIK3CA, MTOR, PIK3R1, TSC1, AKT3, AKT1, TSC2, STK11, and DEPDC5;
CX3CR1, TMEM119, P2RY12; DNMT3A, TET2, ASXL1, KMT2D, ATRX BCR, CBL, TP53, MLH1, and STAT3.

10. A set of probes targeting the exons and exon-intron junctions of proliferation-related genes listed in claim 8.

11. A set of primers for amplifying the gene panels of claim 8.

12. A method of reducing neuroinflammation in a subject, the method comprising contacting a cell of the subject with an agent of Table 1 that inhibits the activity or expression of a proliferation-related polypeptide, cell cycle-related polypeptide, tumor suppressor polypeptide, or a polynucleotide encoding such polypeptides.

13. A kit for characterizing neuroinflammation in a biological sample of a subject, the kit comprising a panel of capture molecules of claim 8.

14. A method of characterizing neuroinflammation in a biological sample of a subject, the method comprising:
a) sequencing RNA present in a biological sample comprising somatic cells of the subject to generate sequencing data;
b) analyzing the sequencing data using the computer, in comparison to a reference sequence, at one or more loci associated with clonal hematopoiesis of indeterminate potential (CHIP), to generate a set of somatic copy number variations associated with CHIP present in the somatic cells; and
c) calculating a burden of somatic copy number variations associated with CHIP in the biological sample relative to a reference subject, wherein an increase in the burden of somatic copy number variations in the subject, relative to the reference sequence, is indicative of neuroinflammation in the biological sample.

15. A computer implemented method of determining a burden of somatic copy number variations in a biological sample of a subject, the method comprising:
a) sequencing RNA present in a biological sample comprising somatic cells of the subject to generate sequencing data;
b) analyzing the sequencing data using the computer, in comparison to a reference sequence, at one or more loci associated with CHIP, to generate a set of somatic copy number variations associated with CHIP present in the somatic cells; and
c) calculating a burden of somatic copy number variations associated with CHIP in the biological sample relative to the reference sequence.

16. A method of characterizing Alzheimer's disease in a biological sample of a subject, the method comprising:
a) sequencing RNA present in a biological sample comprising somatic cells of the subject to generate sequencing data;
b) analyzing the sequencing data using the computer, in comparison to a reference sequence, at one or more loci associated with CHIP, to generate a set of somatic copy number variations associated with CHIP present in the somatic cells; and
c) calculating a burden of somatic copy number variations associated with CHIP in the biological sample relative to a reference subject, wherein an increase in the burden of somatic copy number variations in the subject, relative to the reference sequence, is indicative of Alzheimer's disease or a propensity to develop Alzheimer's disease in the subject.

17. The method of claim 16, wherein the biological sample comprises a microglia-perivascular macrophage, astrocyte, oligodendrocyte, oligodendrocyte precursor cell, excitatory neuron, or cell free DNA derived from any of the aforementioned cell types.

18. The method of claim 17, wherein the biological sample is derived from prefrontal cortex, temporal cortex, or another brain region.

19. A method of characterizing neuroinflammation in a biological sample of a subject, the method comprising:
a) sequencing RNA present in a biological sample comprising somatic cells of the subject to generate sequencing data;
b) analyzing the sequencing data using the computer, in comparison to a reference sequence, at one or more loci associated with CHIP, to generate a set of somatic copy number variations associated with CHIP present in the somatic cells;
c) sorting the sequencing data, using the set of somatic copy number variations associated with CHIP, into sequencing data from cells having copy number variations associated with CHIP (sCNV data) and sequencing data from cells not having copy number variations associated with CHIP (no sCNV data); and
d) comparing expression levels for a set of genes between the sCNV data and the no SCNV data, wherein the set of genes comprises one or more of NEAT1, CD163, SLC11A1, SRGN, ASAP1, CADM1, RGS1, HIF1A, and GLUL, and wherein an increase in expression levels for the set of genes in the sCNV data as compared to the no sCNV data is indicative of neuroinflammation in the biological sample.

20. A method of characterizing Alzheimer's disease in a biological sample of a subject, the method comprising:
a) sequencing RNA present in a biological sample comprising somatic cells of the subject to generate sequencing data;
b) analyzing the sequencing data using the computer, in comparison to a reference sequence, at one or more loci associated with CHIP, to generate a set of somatic copy number variations associated with CHIP present in the somatic cells;
c) sorting the sequencing data, using the set of somatic copy number variations associated with CHIP, into sequencing data from cells having copy number variations associated with CHIP (sCNV data) and sequencing data from cells not having copy number variations associated with CHIP (no sCNV data); and
d) comparing expression levels for a set of genes between the sCNV data and the no SCNV data, wherein the set of genes comprises one or more of NEAT1, CD163, SLC11A1, SRGN, ASAP1, CADM1, RGS1, HIF1A, and GLUL, and wherein an increase in expression levels for the set of genes in the sCNV data as compared to the no sCNV data is indicative of Alzheimer's disease or a propensity to develop Alzheimer's disease in the subject.
